(19) United States
(12) Patent Application Publication
(10) Pub. No.: US 2022/0257595 A1
(43) Pub. Date: Aug. 18, 2022

(54) MACITENTAN FOR USE IN TREATING PORTOPULMONARY HYPERTENSION

(71) Applicant: ACTELION PHARMACEUTICALS LTD, Allschwil (CH)

(72) Inventors: Loïc PERCHENET, Allschwil (CH); Emmanuelle COTTREEL, Allschwil (CH)

(21) Appl. No.: 17/601,123

(22) PCT Filed: Apr. 3, 2020
(86) PCT No.: PCT/EP2020/059499

§ 371 Date: Oct. 4, 2021

Related U.S. Application Data
(60) Provisional application No. 62/830,008, filed on Apr. 5, 2019.

(51) Int. Cl. A61K 31/506; A61P 9/12; A61P 1/16; A61K 45/06

(52) U.S. Cl. CPC A61K 31/506; A61K 45/06; A61P 1/16; A61P 9/12

(57) ABSTRACT

The present invention provides methods for treating portopulmonary hypertension, comprising administering to a patient in need thereof, a therapeutically effective amount of macitentan. Preferably, the methods are clinically proven safe and/or effective. Also provided are methods of improving liver transplant perioperative mortality risk category, improving MELD exception eligibility, and reducing the risk of removal from a liver transplant waitlist in a patient with portopulmonary hypertension and liver disease, comprising administering to a patient in need thereof a therapeutically effective amount of macitentan.

### CROSS-REFERENCE TO RELATED APPLICATIONS

This application claims priority to U.S. Provisional Patent Application No. 62/830,008, filed Apr. 5, 2019, the disclosure of which is incorporated herein by reference in its entirety.

### TECHNICAL FIELD

The present invention relates to methods for treating subjects diagnosed with portopulmonary hypertension.

### BACKGROUND

Portopulmonary hypertension (PoPH) is defined as pulmonary arterial hypertension (PAH) associated with portal hypertension with or without underlying hepatic disease. It belongs to Group I (PAH) of the current WHO classification of pulmonary hypertension (PH). PoPH diagnosis is based on pulmonary hemodynamic criteria for PAH obtained via right heart catheterization (RHC), including mean pulmonary arterial pressure (mPAP) ≤25 mmHg at rest and mean pulmonary artery wedge pressure (PAWP) ≥15 mmHg, within the context of portal hypertension.

The development of PoPH appears to be independent of the cause and severity of portal hypertension but rather as a consequence of portal hypertension in genetically predisposed patients. It is likely that the pathogenic changes observed in PoPH arise from a combination of aberrant vasoactive and angiogenic signaling due to portal hypertension coupled with a hyperdynamic circulatory state, resulting in disruption of the normal hepatopulmonary circulation, increased vascular shear/mechanical stress in the lung vasculature, and thus the pathogenic changes observed in PoPH.

In approximately 88% of patients, portal hypertension is caused by liver cirrhosis (most frequently due to alcohol abuse or viral/autoimmune hepatitis). The prevalence of PoPH among patients with cirrhosis is estimated to be 3-5%. Portal hypertension, and consequently, PoPH, can also occur in the absence of cirrhosis, in the presence of extrahepatic causes such as portal vein occlusion, hepatic vein occlusion or inferior vena cava thrombosis.

PoPH presentation is typically during the fifth decade of life (mean age=49±13 years SD), with no observed differences in prevalence between men and women. On average, PAH is diagnosed 4-7 years after the diagnosis of portal hypertension. The most common presenting symptom is dyspnea on exertion, with other non-specific symptoms such as syncope, chest pain, fatigue, light-headedness, orthopnea, and edema. However, patients often present no specific symptoms indicative of PAH, and PoPH is most commonly diagnosed during evaluation for liver transplantation.

The prognosis for patients with PoPH is poor. While 5-year survival outcomes (historically less than 10%) have recently improved (40% in the US) owing to access to modern PAH-specific pharmacological intervention, the risk of death was 4-fold compared to idiopathic PAH. In France, survival outcomes at 5 years were reported to be comparable to that of idiopathic PAH (IPAH) at 68%. Causes of death reflect the existence of two serious illnesses: advanced liver disease and PAH with right heart failure. These prognostic data are not surprising because PoPH patients generally have advanced liver disease in addition to pulmonary arterial hypertension. In addition, although PoPH patients have similar pulmonary vascular pathophysiology to IPAH, they are less likely to receive PAH-specific therapy compared to patients with other forms of PAH. This could in part be attributed to the paucity of data from clinical studies specifically studying pharmacological therapy in the PoPH population, as these patients have been historically excluded from most randomized clinical trials in PAH.

The immediate goal in the management and treatment of PoPH is to improve pulmonary hemodynamics by reducing the obstruction to pulmonary arterial flow, and thus unloading the right ventricle. The European Respiratory Society (ERS) Task Force on Pulmonary-Hepatic Vascular Disorders recommends the use of intravenous (i.v.) prostacyclin (epoprostenol), prostacyclin analogs (inhaled iloprost), and endothelin receptor antagonists (ERAs; such as bosentan) for PoPH treatment. More recent clinical experience with approved PAH medications, such as the phosphodiesterase type 5 (PDE-5) inhibitor sildenafil, inhaled and i.v. treprostinil (prostacyclin analog), and the ERA ambrisentan suggest beneficial effects of vasodilator therapy in PoPH. However, this PoPH clinical experience has been limited to single-center, open-label (OL) studies, since PoPH patients were routinely excluded from randomized, controlled PAH studies due to the potential of an altered drug metabolism arising from the underlying hepatic disease.

Orthotopic liver transplantation (OLT) is indicated in advanced liver disease, a common feature in portal hypertension, but is contra-indicated in patients with compromised cardiopulmonary hemodynamics, as observed in PoPH. The ERS Task Force, however, recommends that patients with mild PAH (mPAP <35 mmHg) may be eligible for OLT. Additionally, it recommends that patients with moderate PAH (mPAP ≥35-45 mmHg) receive pulmonary vasodilator therapy prior to OLT to improve cardiopulmonary hemodynamics (i.e., lower both mPAP and pulmonary vascular resistance (PVR)). For patients with severe PAH (mPAP >45 mmHg), OLT is contraindicated but chronic vasodilator therapy is recommended to manage PAH.

The presence of moderate to severe pulmonary hypertension in patients with liver disease increases the risk of graft dysfunction and cardiopulmonary-related mortality after OLT. After hepatectomy and graft reperfusion, hemodynamic changes lead to marked increases in right ventricular preload. Given that the pulmonary vasculature in PoPH is a relatively low compliance and high resistance circuit, this increase in cardiac output can acutely raise the pulmonary arterial pressure significantly to precipitate acute right ventricular failure and death. Within the first year post-transplant, PoPH patients have a significantly higher risk of death and graft failure compared to other liver transplant recipients.

Since both cirrhosis and PAH are progressive conditions, in PoPH one of the options is to perform the liver transplant before the hemodynamic state is too severely impaired for the procedure. In the US, a PoPH model for end-stage liver disease (MELD) exception rule (MELD exception) is in place to facilitate access to a liver graft to patients with PoPH who attain mPAP ≤35 mmHg and PVR ≤400 dyn·sec·cm⁻⁵ with PAH treatment. Patients are priority-ranked on the waitlist with a higher MELD score than their actual score. This allows obtaining a liver graft at a time when liver impairment is moderate. Alternatively, the patient waits until the real MELD score is high enough to obtain a liver graft. The inherent risk is that the hemodynamics might then be so severely impaired that transplant will be contraindicated.

Macitentan (Opsumit®), approved for the treatment of PAH at 10 mg dose, is an orally active, non-peptide, potent dual ERA (targeting both endothelin A and B receptors) that demonstrated higher potency than bosentan in nonclinical in vivo studies. Phase 1 clinical data in chronic hepatic impairment showed no clinically relevant change in the pharmacokinetics (PK) of macitentan and its metabolites in varying grades of liver disease severity.

Prior nonclinical and clinical studies have shown macitentan treatment to be associated with teratogenicity, dose-related anemia/decrease in hemoglobin concentration, and hepatotoxicity. To date, there have been no randomized, controlled clinical studies testing the safety and efficacy of targeted PAH therapy in patients with PoPH.

Despite the current standard of care, there are no treatments for subjects with PoPH; instead such patients have been managed with commercially available PAH medications including ERAs. What is needed are methods of treating patients having PoPH, including those also having underlying hepatic disease.

### SUMMARY

In some embodiments, the disclosure provides methods for treating portopulmonary hypertension, comprising administering to a patient in need thereof, a therapeutically effective amount of macitentan. Preferably, the methods are clinically proven safe and/or effective.

In further embodiments, the disclosure provides methods of improving liver transplant perioperative mortality risk category in a patient with portopulmonary hypertension and liver disease, comprising administering to a patient in need thereof, a therapeutically effective amount of macitentan.

In other embodiments, the disclosure provides methods of improving MELD exception eligibility in a patient with portopulmonary hypertension and liver disease, comprising administering to a patient in need thereof a therapeutically effective amount of macitentan. In further embodiments, the disclosure includes methods of reducing the risk of removal from a liver transplant waitlist due to mortality or clinical deterioration in a patient with portopulmonary hypertension and liver disease, comprising administering to a patient in need thereof, a therapeutically effective amount of macitentan. Preferably, the methods are clinically proven safe and/or effective.

In yet further embodiments, the disclosure provides methods of selling a drug product comprising macitentan, said method comprising selling the drug product, wherein a drug product label for a reference listed drug for the drug product includes instructions for treating portopulmonary hypertension.

In still further embodiments, the disclosure provides methods of offering for sale a drug product comprising macitentan, said method comprising offering for sale such drug product, wherein a drug product label for a reference listed drug for such drug product includes instructions for treating portopulmonary hypertension.

In other embodiments, the disclosure provides methods of treating portopulmonary hypertension, comprising administering to a patient in need thereof an approved drug product comprising macitentan in an amount described in a drug product label for the drug product.

In further embodiments, the disclosure provides pharmaceutical drug products comprising a clinically proven safe and clinically proven effective amount of macitentan, wherein the pharmaceutical product is packaged and wherein the package includes a label that identifies macitentan as a regulatory approved chemical entity, and instructs use of macitentan for the treatment of portopulmonary hypertension.

### BRIEF DESCRIPTION OF THE DRAWINGS

FIG. 1A is a schematic of the disposition of patients. In this figure, all patients randomized in the DB period of the study received DB study treatment.

FIG. 1B depicts the study design.

FIG. 2 is a schematic showing the PK sub-study design.

FIG. 3 is a scatter plot of PVR at Week 12 versus baseline, full analysis set.

FIG. 4 is a Forest plot of change from baseline to Week 12 in PVR per subgroup and overall, full analysis set. In this figure, n(trt)=number of subjects in macitentan and n(pla)=number of subjects in placebo. The vertical solid line references the overall treatment effect. Square size for subgroups are based on number of subjects. Region and PAH therapy are as per IXRS. Eosophageal varices are as per “portal Hypertension Relevant Disease History’ CRF page. P-values reflect treatment-by-subgroup variable interaction testing on extended main model (including other stratification factor and baseline as covariate) with subgroup variable effect and its interaction with treatment added.

FIG. 5 is a line graph of the model-adjusted mean (95% CLs) change in 6MWD from baseline to Weeks 4, 8, and 12, Full analysis set.

FIG. 6 is a line graph of the mean (95% CLs) change from baseline in 6MWD to Weeks 4, 8, 12, 16, 20 and 24 (observed cases only), full analysis set.

FIG. 7 is a Forest plot of changes from baseline to Week 12 in 6MWD per subgroup and overall (MMRM analysis), full analysis set. In this figure, n(trt)=Number of subjects in Macitentan and n(pla)=Number of Subjects in Placebo. The vertical solid line references the overall treatment effect. Square size for subgroups are based on number of subjects. Region and PAH therapy are as per IXRS. Eosophageal varices are as per “portal Hypertension Relevant Disease History’ CRF page. P-values reflect treatment-by-subgroup variable interaction testing on extended main model (including other stratification factor and baseline as covariate) with subgroup variable effect and its interaction with treatment added.

FIGS. 8A and 8B are macitentan and ACT-132577 PK profiles in PAH (8A) and PoPH (8B) patients, respectively.

FIG. 9 is a dot plot showing the 35% reduction in PVR for macitentan treated patients vs. placebo.

FIG. 10 is a schematic of the PORTICO PK sub-study design, showing the PK sampling.

FIG. 11 is a scatterplot of mPAP change from baseline at week 12 versus baseline, full analysis set.

FIG. 12 is a scatterplot of PVR change from baseline at week 12 versus baseline, full analysis set.

### DETAILED DESCRIPTION OF ILLUSTRATIVE EMBODIMENTS

In the present disclosure the singular forms “a”, “an,” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about” or “substantially” it will be understood that the particular value forms another embodiment. In general, use of the term “about” or “substantially” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about” or “substantially”. In other cases, the gradations used in a series of values may be used to determine the intended range available to the term “about” or “substantially” for each value. Where present, all ranges are inclusive and combinable. That is, references to values stated in ranges include every value within that range.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiments and such a combination is considered to be another embodiment. Conversely, various features of the invention that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself.

### Methods

The present disclosure provides methods for treating portopulmonary hypertension. In some aspects, the portopulmonary hypertension is caused by a liver disease. In certain aspects, the liver disease is caused by cirrhosis, Hepatitis B, Hepatitis C, alcoholism, autoimmune hepatitis, primary biliary cirrhosis, or cryptogenic cirrhosis. In other aspects, the portopulmonary hypertension is caused by portal vein occlusion, hepatic vein occlusion, or inferior vena cava thrombosis. In other aspects, the methods comprise determining if the patient has portopulmonary hypertension and, if so, administering to the patient a therapeutically effective amount of macitentan. Portopulmonary hypertension diagnostic workup includes one or more of an echocardiogram, chest X-ray, electrocardiogram, blood tests, computerized tomography scan, magnetic resonance imaging, pulmonary function test, polysomnogram, ventilation/perfusion scan, or open-lung biopsy. Final diagnostic tests include right heart catheterization, without limitation.

As used herein, the terms “portopulmonary hypertension” and “PoPH” are interchangeable and refer to pulmonary arterial hypertension (PAH) associated with portal hypertension. In some embodiments, the portal hypertension is secondary to an underlying liver disease. In other embodiments, the portal hypertension is not secondary to an underlying liver disease. Patients having PoPH may have a mean pulmonary arterial pressure (mPAP) of about 25 mmHg or greater (i.e., >25 mmHg) at rest. In some embodiments, the patients have a mPAP at rest of about 25, about 26, about 27, about 28, about 29, about 30, about 31, about 32, about 33, about 34, about 35, about 36, about 37, about 38, about 39, about 40, about 41, about 42, about 43, about 44, or about 45 mmHg at rest. In further embodiments, the patients have a mPAP at rest of about 25 to about 45, about 25 to about 40, about 25 to about 35, about 25 to about 30, about 30 to about 45, about 30 to about 40, about 30 to about 35, about 35 to about 45, about 35 to about 40, about 40 to about 45 mmHg at rest. In other embodiments, the patients have a mPAP at rest of about 25 mmHg to less than about 45 mmHg.

Patients with PoPH may also have a mean pulmonary arterial wedge pressure (PAWP) of less than about 15 mmHg. In some embodiments, patients may have a mean pulmonary arterial wedge pressure of less than about 15, about 14, about 13, about 12, about 11, about 10, about 9, about 8, about 7, about 6, about 5, about 4, about 3, about 2, or about 1 mmHg. In other embodiments, patients may have a mean pulmonary arterial wedge pressure of about 1 to about 15, about 1 to about 10, about 1 to about 5, about 5 to about 15, about 5 to about 10, about 10 to about 15 mmHg.

Patients with PoPH may also have a pulmonary vascular resistance (PVR) of at least about 3 WU (Wood Units), i.e., at least about 240 dyn·s·cm⁻⁵. In some embodiments, the PVR is at least about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, or about 600 dyn·s·cm⁻⁵. In other embodiments, the PVR is about 240 to about 600, about 240 to about 600, about 240 to about 550, about 240 to about 500, about 240 to about 450, about 300 to about 600, about 310 to about 550, about 300 to about 500, about 300 to about 450, about 350 to about 600, about 350 to about 550, about 350 to about 500, about 350 to about 450, about 350 to about 400, about 400 to about 600, about 400 to about 550, about 400 to about 500, about 400 to about 450, about 450 to about 600, about 450 to about 550, about 450 to about 500, about 500 to about 600, about 500 to about 550, or about 550 to about 600 dyn·s·cm⁻⁵. In some embodiments, the patient has a PVR of about 5 Wood units (about 450 dyn·s·cm⁻⁵) or greater. In other embodiments, the patient has a PVR of about 4 Wood units (about 320 dyn·s·cm⁻⁵) or greater.

The patient may also be capable of performing a 6-minute walk test with a distance of at least 50 m, does not have Child-Pugh class C liver disease, and/or does not have a Model for End-Stage Liver Disease (MELD) score of 19. In some embodiments, the patient is capable of performing a 6-minute walk test with a distance of at least 50 m. In other embodiments, the patient does not have Child-Pugh class C liver disease or a Model for End-Stage Liver Disease (MELD) score of 19. In further embodiments, the patient does not have Child-Pugh class C liver disease. In still other embodiments, the patient does not have a Model for End-Stage Liver Disease (MELD) score of 19.

As described herein, “MELD score” may be calculated by those skilled in the art. In some embodiments, calculation of the MELD score uses the patient's laboratory results or a fixed serum creatinine value of 4.0 mg/dL instead of the laboratory results if patient was dialyzed twice in the past week. The MELD score is based on a numerical scale, ranging from 6 (less ill) to 40 (gravely ill), used for liver transplant candidates age 12 and older. It gives each person a ‘score’ (number) based on how urgently a liver transplant is needed within the next three months. The number is calculated by a formula using three routine lab test results including bilirubin, which measures how effectively the liver excretes bile; INR (prothrombin time), which measures the liver's ability to make blood clotting factors; and creatinine, which measures kidney function. Several online tools are available for calculating the MELD score (e.g., original MELD score). In other embodiments, the original MELD score is calculated by the United Network for Organ Sharing (UNOS) as follows:

$$MELD = 3.8 * {log}_{e} ( serum bilirubin [ \frac{mg}{dL} ] ) + 11.2 * {log}_{e} ( I N R ) + 9.6 * {log}_{e} \\ ( serum creatinine [ \frac{mg}{dL} ] ) + 6. 4$$

In an effort to eliminate negative values, any measured laboratory values that are less than 1.0 are assigned a value of 1.0. For example, patients with the combination of an INR of ≤1, serum creatinine ≤1 mg/dL, and serum bilirubin ≤1 mg/dL receive the minimum MELD score of 6. Further, in an effort to avoid an unfair advantage for patients with intrinsic renal disease, the maximum serum creatinine level is set to 4.0 mg/dL, which is also the value that is automatically assigned to patients who have received hemodialysis at least twice, or continuous venovenous hemodialysis for 24 hours, in the preceding week. There is no modification in the score for patients receiving anticoagulation.

The methods include administering a therapeutically effective amount of the macitentan. The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In some embodiments, the therapeutically effective amount of macitentan is less than about 15 mg. In further embodiments, the therapeutically effective amount of macitentan is about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, or about 15 mg. In other embodiments, the therapeutically effective amount of macitentan is about 1 to about 15 mg, about 1 to about 10 mg, about 1 to about 5 mg, about 5 to about 15 mg, about 5 to about 10 mg, or about 10 to about 15 mg. In yet further embodiments, the therapeutically effective amount is about 5 to about 15 mg. In yet further embodiments, the therapeutically effective amount is about 10 mg.

The methods described herein are effective in the lowering of one or both of mPAP and PVR. In some embodiments, the methods reduce mPAP. Thus, the methods result in mPAP levels of about 35 mmHg or less. In some embodiments, the methods result in mPAP levels of about 35, about 30, about 25, about 20, about 15, about 10, or about 5 mmHg or less.

The methods also are effective to reduce PVR. In some embodiments, the PVR is reduced by at least about 30% relative to a patient at the same level of disease diagnosis (placebo group) that is not receiving treatment with macitentan. In other embodiments, the methods result in the lowering of mPAP levels of about 1 to about 35, about 1 to about 30 about 1 to about 25, about 1 to about 20, about 1 to about 20, about 1 to about 15, about 1 to about 10, about 1 to about 5, about 5 to about 35, about 5 to about 30, about 5 to about 25, about 5 to about 20, about 5 to about 15, about 5 to about 10, about 10 to about 35, about 10 to about 30, about 10 to about 25, about 10 to about 20, about 10 to about 15, about 15 to about 35, about 15 to about 30, about 15, to about 25, about 15 to about 20, about 20 to about 35, about 20 to about 30, about 20 to about 25, about 25 to about 35, about 25 to about 30, or about 30 to about 35 mmHg. In further embodiments, the methods result in lowering the PVR to less than about 400 dyn·s·cm⁻⁵. In yet other embodiments, the methods result in lowering the PVR to less than about 400, about 350, about 300, about 250, about 200, about 150, about 100, about 75, or about 50 dyn·s·cm⁻⁵. In yet further embodiments, the methods result in lowering the mPAP to 35 mmHg or less and the PVR to 400 dyn·sec·cm⁻⁵ or less which is referenced herein as the MELD exception.

In further embodiments, the methods reduce mPAP and PVR. Thus, the methods are effective in reducing mPAP and PVR following administration of macitentan.

The present disclosure also provides methods of improving liver transplant perioperative mortality risk category in a patient with portopulmonary hypertension and liver disease. For example, a patient in a high risk category has a mPAP ≥45 mmHg and a liver transplant is contraindicated. A patient in an intermediate risk category has a mPAP ≤35 mmHg and <45 mmHg, with a low risk category defined as a mPAP <35 mmHg. The methods described herein result in a patient moving to a lower risk category following treatment with macitentan.

In other aspects, the present disclosure provides methods for improving MELD exception eligibility in a patient with portopulmonary hypertension and liver disease, comprising administering to a patient in need thereof, a therapeutically effective amount of macitentan. The MELD exception eligibility criteria comprises a mPAP ≤35 mmHg and a PVR ≤400 dyn·sec·cm⁻⁵. The methods disclosed herein result in a patient outside the MELD exception eligibility criteria subsequently meeting the MELD exception eligibility criteria. Such eligibility facilitates a patient's access to a liver graft.

The methods of the present disclosure also include methods of reducing the risk of removal from a liver transplant waitlist due to mortality or clinical deterioration in a patient with portopulmonary hypertension and liver disease, comprising administering to a patient in need thereof, a therapeutically effective amount of macitentan. For example, a patient with a PVR >450 dyn·s·cm⁻⁵ has a high risk of removal from the liver transplant waiting list due to mortality or clinical deterioration. The methods of the present disclosure result in the patient reducing the risk of removal from the liver transplant waitlist by lowering the PVR to ≤400 dyn·sec·cm⁻⁵.

Typically, the methods of the present invention comprise about twelve weeks of treatment with macitentan. In other aspects, the patient receives background pulmonary arterial hypertension (PAH) specific therapy comprising, for example, one or more of a phosphodiesterase type 5 inhibitor, a soluble guanylate cyclase stimulator, or an inhaled prostanoid. In some embodiments, the background pulmonary arterial hypertension specific therapy is present for at least three months at a stable dose prior to administration of macitentan. Improvements in liver transplant perioperative mortality risk category, MELD exception eligibility, and the reduction of risk of removal from a liver transplant waitlist are seen even in patients receiving such background PAH specific therapy.

Desirably, the methods of administering macitentan, as described herein, do not substantially affect hepatic venous pressure gradient and/or systolic blood pressure of the patient. In certain embodiments, the administration of macitentan does not substantially affect hepatic venous pressure gradient. In other embodiments, the administration of macitentan does not substantially affect systolic blood pressure of the patient. The methods also result in increasing the cardiac index of the patient following administration of macitentan.

As used herein, unless otherwise noted, the term “macitentan” refers to N-[5-(4-Bromophenyl)-6-[2-[(5-bromo-2-pyrimidinyl)oxy]ethoxy]-4-pyrimidinyl]-N′-propylsulfamide of formula (I).

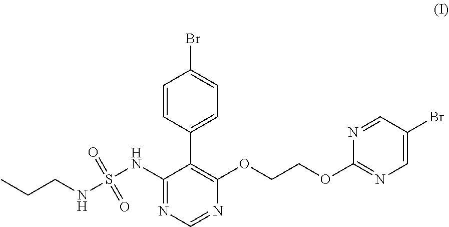 [SMILES: CCCNS(=O)(=O)Nc1ncnc(OCCOc2ncc(Br)cn2)c1-c1ccc(Br)cc1]

In other embodiments, macitentan refers to stereoisomers of macitentan, such as enantiomers and diastereomers as pure or substantially pure forms. Macitentan also refers to racemic mixtures thereof.

As used herein, “macitentan” also refers to amorphous or crystalline forms of macitentan. In some embodiments, the macitentan is a crystalline form. In other embodiments, the macitentan is an amorphous form. The crystallinity may be determined by those skilled in the art using one or more techniques such as, e.g., single crystal x-ray diffraction, powder x-ray diffraction, differential scanning calorimetry, melting point, among others.

“Macitentan” as used herein includes anhydrous forms or hydrates thereof. In certain embodiments, the macitentan is in an anhydrous form. In other embodiments, the macitentan is a hydrate thereof. “Macitentan” as used herein further refers to solvates thereof. Such solvates include a molecule of a solvent bound through intermolecular forces or chemical bonds to one or more locations of the macitentan molecule.

As used herein, “macitentan” may also refer to polymorphs thereof. Such polymorphs of macitentan include crystalline forms of the molecule, having variations to the crystal lattices of each polymorph.

The term “macitentan” may also include pharmaceutically acceptable salts thereof, which may readily be selected by those skilled in the art. The expression pharmaceutically acceptable salts encompasses either salts with inorganic acids or organic acids like hydrohalogenic acids, e.g. hydrochloric or hydrobromic acid; sulfuric acid, phosphoric acid, nitric acid, citric acid, formic acid, acetic acid, maleic acid, tartaric acid, methylsulfonic acid, p-toluolsulfonic acid and the like or in case the compound of formula I is acidic in nature with an inorganic base like an alkali or earth alkali base, e.g. sodium hydroxide, potassium hydroxide, calcium hydroxide and the like.

Macitentan is commercially available as understood to those skilled in the art. For example, macitentan is available as OPSUMIT®. Macitentan is an endothelin receptor antagonist and may be prepared according to the process as disclosed in U.S. Pat. No. 7,094,781, which is incorporated by reference herein.

The present invention also contemplates the administration of macitentan metabolites. Desirably, the macitentan metabolite is metabolically active compound. Thus, in certain embodiments, the macitentan metabolite is of formula M1-M7. In some embodiments, the macitentan metabolite is of formula M6; M6 is also known under the code name ACT-132577 and the International non-proprietary name aprocitentan.

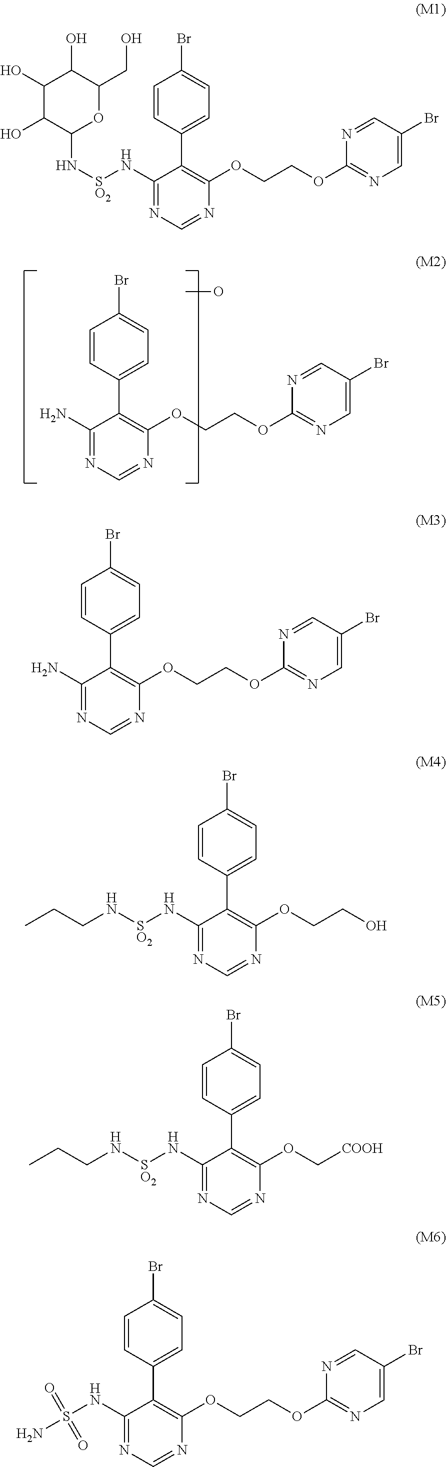

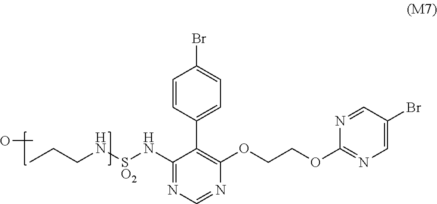

As used herein, unless otherwise noted, the terms “treating”, “treatment” and the like, shall include the management and care of a patient for the purpose of combating a disease, condition, or disorder. The terms “treating” and “treatment” also include the administration of the compounds or pharmaceutical compositions as described herein to (a) alleviate one or more symptoms or complications of the disease, condition or disorder; (b) prevent the onset of one or more symptoms or complications of the disease, condition or disorder; and/or (c) eliminate one or more symptoms or complications of the disease, condition, or disorder.

As used herein, unless otherwise noted, the terms “preventing”, “prevention” and the like, shall include (a) reducing the frequency of one or more symptoms; (b) reducing the severity of one or more symptoms; (c) delaying, slowing or avoiding of the development of additional symptoms; and/or (d) slowing, or avoiding the development of the disorder or condition to a later stage or more serious form.

One skilled in the art will recognize that, wherein the present disclosure is directed to methods of prevention, a patient in need thereof shall include any patient who has experienced or exhibited at least one symptom of the disorder, disease or condition to be prevented. Further, a patient in need thereof may additionally be a patient who has not exhibited any symptoms of the disorder, disease or condition to be prevented, but who has been deemed by a physician, clinician or other medical profession to be at risk of developing said disorder, disease or condition. For example, the patient may be deemed at risk of developing a disorder, disease or condition (and therefore in need of prevention or preventive treatment) as a consequence of the patient's medical history, including, but not limited to, family history, pre-disposition, co-existing (comorbid) disorders or conditions, genetic testing, and the like.

The terms “subject” and “patient” are interchangeably used herein to refer to an animal, preferably a mammal, most preferably a human, who has been the object of treatment.

The methods also permit administering a concomitant standard of care or background therapy. The term “standard of care” typically refers to a physician prescribed treatment of the disease condition at issue. In some embodiments, the standard of care comprises, consists of, or consists essentially of administering an additional pharmaceutical agent that is an α- or β-blocker such as phentolamine, phenoxybenzamine, atenolol, propranolol, timolol, metoprolol, carteolol and the like; a vasodilator such as hydralazine, minoxidil, diazoxide or flosequinan; a calcium-antagonist such as diltiazem, nicardipine, nimodipine, verapamil or nifedipine; a ACE-inhibitor such as cilazapril, captopril, enalapril, or lisinopril; a potassium activator such as pinacidil; an angiotensin II receptor antagonist such as losartan, valsartan, or irbesartan; a diuretic such as hydrochlorothiazide, chlorothiazide, acetolamide, bumetanide, furosemide, metolazone or chlortalidone; a sympatholytic such as methyldopa, clonidine, guanabenz or reserpine; or another therapeutic which serves to treat high blood pressure or any cardiac disorder. Other PAH therapy comprises PDE-5 inhibitors, soluble guanylate cyclase (sGC) stimulators, or inhaled prostanoid therapy. Typically, the standard of care does not include treatment by administering macitentan. The standard of care may be administered to the patient prior to, subsequently to, or concurrently with macitentan. In some embodiments, the standard of care is administered before macitentan. In other embodiments, the standard of care is administered after macitentan. In further embodiments, the standard of care is administered concurrently with macitentan. In yet other embodiments, the standard of care is present for at least three months at a stable dose prior to administration of macitentan.

In the methods described herein, the therapeutically effective amount of macitentan is safe, effective, or safe and effective. As used herein, unless otherwise noted, the term “safe” shall mean without undue adverse side effects (such as toxicity, irritation, or allergic response), commensurate with a reasonable benefit/risk ratio when used in the manner of this invention. Similarly, unless otherwise noted, the term “effective” means the efficacy of treatment has been demonstrated for the treatment of patients with portopulmonary hypertension when dosed in a therapeutically effective dose. In certain embodiments, the methods described herein are safe. In other embodiments, the methods described herein are effective. In further embodiments, the methods described herein are safe and effective. In yet other embodiments, the therapeutically effective amount of macitentan is safe. In still further embodiments, the therapeutically effective amount of macitentan is effective. In other embodiments, the therapeutically effective amount of macitentan is safe and effective.

As used herein, unless otherwise noted, the term “clinically proven” (used independently or to modify the terms “safe” and/or “effective”) shall mean that proof has been proven by a Phase III or IV clinical trial that are sufficient to meet approval standards of U.S. Food and Drug Administration or similar study for market authorization by EMEA. Preferably, an adequately sized, randomized, double-blinded controlled study is used to clinically prove the effects of macitentan as compared to a placebo with the patient's condition assessed by techniques described herein.

As used herein, unless otherwise noted, the term “clinically proven effective” means the efficacy of treatment has been proven by a Phase III or IV clinical trial as statistically significant i.e., the results of the clinical trial are not likely to be due to chance with an alpha level less than 0.05 or the clinical efficacy results are sufficient to meet approval standards of U.S. Food and Drug Administration or similar study for market authorization by EMEA. For example, macitentan was clinically proven effective for the treatment of patients with portopulmonary hypertension in a therapeutically effective dose as described herein, and as specifically set forth in the examples.

As used herein, unless otherwise noted, the term “clinically proven safe” means the safety of treatment has been proven by a Phase III or IV clinical trial by analysis of the trial data and results establishing that the treatment is without undue adverse side effects and commensurate with the statistically significant clinical benefit (e.g., efficacy) sufficient to meet approval standards of U.S. Food and Drug Administration or similar study for market authorization by Europe, the Middle East, and Africa (EMEA). For example, macitentan was clinically proven safe for the treatment of patients with portopulmonary hypertension when dosed in a therapeutically effective dose as described herein, and as specifically set forth in the examples.

In certain aspects, methods of selling a drug product comprising macitentan are also provided. The terms “sale” or “selling” as used herein refers to transferring a drug product, e.g., a pharmaceutical composition or a dosage form, from a seller to a buyer. Thus, the methods include selling a drug product comprising macitentan, wherein the method comprises selling the drug product. In some embodiments, a drug product label for a reference listed drug for the drug product includes instructions for treating portopulmonary hypertension. The methods also include offering for sale a drug product comprising macitentan. The term “offering for sale,” as used herein, refers to the proposal of a sale by a seller to a buyer for a drug product, e.g., a pharmaceutical composition or a dosage form. These methods comprise offering the drug product for sale.

The term “drug product” refers to a product that contains an active pharmaceutical ingredient that has been approved for marketing by a governmental authority, e.g., the Food and Drug Administration or the similar authority in other countries. In some embodiments, the drug product comprises macitentan.

Similarly, “label” or “drug product label” refers to information provided to a patient which provides relevant information regarding the drug product. Such information includes, without limitation, one or more of the description of the drug, clinical pharmacology, indications (uses for the drug product), contraindication (who should not take the drug product), warnings, precautions, adverse events (side effects), drug abuse and dependence, dosage and administration, use in pregnancy, use in nursing mothers, use in children and older patients, how the drug is supplied, safety information for the patient, or any combination thereof. In certain embodiments, the label or drug product label provides an instruction for use in a patient with portopulmonary hypertension. In further embodiments, the label or drug product label identifies macitentan as a regulatory approved chemical entity. In still other embodiments, the label comprises data for reducing PVR relative to a placebo. In yet further embodiments, the label provides a definition of portopulmonary hypertension and instructs a patient or a physician to administer the macitentan if the patient has portopulmonary hypertension. In other embodiments, the label comprises data or instructions for improving liver transplant perioperative mortality risk category, improving MELD exception eligibility, or reducing the risk of removal from a liver transplant waiting list. In further embodiments, the label provides an instruction for attaining a mPAP ≤35 mmHg and a PVR ≤400 dyn·sec·cm⁻⁵.

The term “reference listed drug” or “RLD” as used herein refers to a drug product to which new generic versions are compared to show that they are bioequivalent. It is also a medicinal product that has been granted marketing authorization by a member state of the European Union or by the Commission on the basis of a completed dossier, i.e., with the submission of quality, pre-clinical and clinical data in accordance with Articles 8(3), 10a, 10b or 10c of Directive 2001/83/EC and to which the application for marketing authorization for a generic/hybrid medicinal product refers, by demonstration of bioequivalence, usually through the submission of the appropriate bioavailability studies.

In certain embodiments, the drug product is an ANDA drug product, a supplemental New Drug Application drug product, or a 505(b)(2) drug product. In the United States, a company seeking approval to market a generic equivalent must refer to the RLD in its Abbreviated New Drug Application (ANDA). For example, an ANDA applicant relies on the FDA's finding that a previously approved drug product, i.e., the RLD, is safe and effective, and must demonstrate, among other things, that the proposed generic drug product is the same as the RLD in certain ways. Specifically, with limited exceptions, a drug product for which an ANDA is submitted must have, among other things, the same active ingredient(s), conditions of use, route of administration, dosage form, strength, and (with certain permissible differences) labeling as the RLD. The RLD is the listed drug to which the ANDA applicant must show its proposed ANDA drug product is the same with respect to active ingredient(s), dosage form, route of administration, strength, labeling and conditions of use, among other characteristics. In the electronic Orange Book, there is a column for RLDs and a column for reference standards. In the printed version of the Orange Book, the RLDs and reference standards are identified by specific symbol.

In Europe, Applicants identify in the application form for its generic/hybrid medicinal product, which is the same as an ANDA or supplemental NDA (sNDA) drug product, the reference medicinal product (product name, strength, pharmaceutical form, marketing authorization holder (MAH, first authorization, Member State/Community), which is synonymous with a RLD, as follows:
1. The medicinal product that is or has been authorized in the European Economic Area (EEA), used as the basis for demonstrating that the data protection period defined in the European pharmaceutical legislation has expired. This reference medicinal product, identified for the purpose of calculating expiry of the period of data protection, may be for a different strength, pharmaceutical form, administration route or presentation than the generic/hybrid medicinal product.
2. The medicinal product, the dossier of which is cross-referred to in the generic/hybrid application (product name, strength, pharmaceutical form, MAH, marketing authorization number). This reference medicinal product may have been authorized through separate procedures and under a different name than the reference medicinal product identified for the purpose of calculating expiry of the period of data protection. The product information of this reference medicinal product will, in principle, serve as the basis for the product information claimed for the generic/hybrid medicinal product.
3. The medicinal product (product name, strength, pharmaceutical form, MAH, Member State of source) used for the bioequivalence study(ies) (where applicable).

The different abbreviated approval pathways for drug products under the Food, Drug, and Cosmetics (FD&C) Act are the abbreviated approval pathways described in sections 505(j) and 505(b)(2) of the FD&C Act (21 U.S.C. 355(j) and 21 U.S.C. 355(b)(2), respectively).

According to the FDA (“Determining Whether to Submit an ANDA or a 505(b)(2) Application Guidance for Industry,” U.S. Department of Health and Human Services, October 2017, pp. 1-14, the contents of which is incorporated herein by reference), NDAs and ANDAs can be divided into the following four categories:
(1) A “stand-alone NDA” is an application submitted under section 505(b)(1) and approved under section 505(c) of the FD&C Act that contains full reports of investigations of safety and effectiveness that were conducted by or for the applicant or for which the applicant has a right of reference or use.
(2) A section 505(b)(2) application is an NDA submitted under section 505(b)(1) and approved under section 505(c) of the FD&C Act that contains full reports of investigations of safety and effectiveness, where at least some of the information required for approval comes from studies not conducted by or for the applicant and for which the applicant has not obtained a right of reference or use.
(3) An ANDA is an application for a duplicate of a previously approved drug product that was submitted and approved under section 505(j) of the FD&C Act. An ANDA relies on the FDA's finding that the previously approved drug product, i.e., the reference listed drug (RLD), is safe and effective. An ANDA generally must contain information to show that the proposed generic product (a) is the same as the RLD with respect to the active ingredient(s), conditions of use, route of administration, dosage form, strength, and labeling (with certain permissible differences) and (b) is bioequivalent to the RLD. An ANDA may not be submitted if studies are necessary to establish the safety and effectiveness of the proposed product.
(4) A petitioned ANDA is a type of ANDA for a drug product that differs from the RLD in its dosage form, route of administration, strength, or active ingredient (in a product with more than one active ingredient) and for which FDA has determined, in response to a petition submitted under section 505(j)(2)(C) of the FD&C Act (suitability petition), that studies are not necessary to establish the safety and effectiveness of the proposed drug product.

A scientific premise underlying the Hatch-Waxman Act is that a drug product approved in an ANDA under section 505(j) of the FD&C Act is presumed to be therapeutically equivalent to its RLD. Products classified as therapeutically equivalent can be substituted with the full expectation that the substituted product will produce the same clinical effect and safety profile as the prescribed product when administered to patients under the conditions specified in the labeling. In contrast to an ANDA, a section 505(b)(2) application allows greater flexibility as to the characteristics of the proposed product. A section 505(b)(2) application will not necessarily be rated therapeutically equivalent to the listed drug it references upon approval.

Thus, the methods of treating portopulmonary hypertension include administering to a patient in need thereof an approved drug product comprising macitentan in an amount described in a drug product label for said drug product.

The methods may also comprise, consist of, or consist essentially of placing macitentan into the stream of commerce. In certain embodiments, the macitentan includes a package insert that contains instructions for safely and effectively treating portopulmonary hypertension using the macitentan.

In further aspects, described herein are methods of selling a pharmaceutical composition containing macitentan comprising, consisting of, or consisting essentially of placing the pharmaceutical composition into the stream of commerce. In certain embodiments, the pharmaceutical composition includes a package insert that contains instructions for safely and effectively treating portopulmonary hypertension using macitentan.

In still further aspects, described herein are methods of offering for sale macitentan comprising, consisting of, or consisting essentially of offering to place the macitentan into the stream of commerce. In certain embodiments, the macitentan includes a package insert that contains instructions for safely and effectively treating portopulmonary hypertension using macitentan.

### Formulations/Compositions

Pharmaceutical compositions containing macitentan as the active ingredient can be prepared by intimately mixing the compound or compounds with a pharmaceutical carrier according to conventional pharmaceutical compounding techniques. As used herein, the terms “composition” and “formulation” are used interchangeably and encompass a product comprising the specified ingredients in the specified amounts, as well as any product, such as a pharmaceutical product, which results, directly or indirectly, from combinations of the specified ingredients in the specified amounts. A summary of pharmaceutical compositions can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

The pharmaceutical compositions or pharmaceutical drug products may be administered by a number of routes as determined by those skilled in the art. Preferably, the pharmaceutical compositions or drug products are administered by route that is suitable for macitentan. In some embodiments, the pharmaceutical compositions or drug products are administered orally, rectally, parenterally, e.g., by intravenous, intramuscular, subcutaneous, intrathecal or transdermal administration or sublingually or as ophthalmic preparation or administered as aerosol. Examples of applications are capsules, tablets, orally administered suspensions or solutions, suppositories, injections, eye-drops, ointments or aerosols/nebulizers.

Preferred applications are intravenous, intra-muscular, or oral administrations as well as eye drops. The dosage used depends upon the type of the specific active ingredient, the age and the requirements of the patient and the kind of application. Generally, dosages of 0.001-0.25 mg/kg body weight per day are considered for an average body weight of about 70 kg. The preparations with compounds can contain inert or as well pharmacodynamically active excipients. Tablets or granules, for example, could contain a number of binding agents, filling excipients, carrier substances or diluents.

These compositions may be administered in enteral or oral form e.g. as tablets, dragees, gelatin capsules, emulsions, solutions or suspensions, in nasal form like sprays or rectally in form of suppositories. These compounds may also be administered intramuscularly, parenterally or intravenously, e.g. in form of injectable solutions.

These pharmaceutical compositions may contain the compounds of formula I as well as their pharmaceutically acceptable salts in combination with inorganic and/or organic excipients which are usual in the pharmaceutical industry like lactose, maize or derivatives thereof, talcum, stearinic acid or salts of these materials.

For gelatin capsules vegetable oils, waxes, fats, liquid or half-liquid polyols may be used. For the preparation of solutions and syrups e.g. water, polyols, saccharose, glucose can be used. Injectables can be prepared by using e.g. water, polyols, alcohols, glycerin, vegetable oils, lecithin or liposomes. Suppositories may be prepared by using natural or hydrogenated oils, waxes, fatty acids (fats), liquid or half-liquid polyols.

The compositions may contain in addition preservatives, stability improving substances, viscosity improving or regulating substances, solubility improving substances, sweeteners, dyes, taste improving compounds, salts to change the osmotic pressure, buffer or anti-oxidants.

To prepare such pharmaceutical compositions, macitentan, as the active ingredient, is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, from about 1 mg to about 15 mg of macitentan or any amount or range therein (preferably about 1 mg, about 5 mg, about 10 mg, or about 15 mg, more preferably about 10 mg) of macitentan. The dosages, however, may be varied depending upon the requirement of the patients, the severity of the condition being treated and the compound being employed. The use of either daily administration or post-periodic dosing may be employed.

Preferably, the pharmaceutical compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. For preparing solid compositions such as tablets, the principal active ingredient (e.g., macitentan) is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present disclosure, or a pharmaceutically acceptable salt thereof. In certain embodiments, two active ingredients can be formulated together, e.g., in a bi-layer tablet formulation. When referring to these preformulation compositions as homogeneous, it is meant that the active ingredients are dispersed evenly throughout the composition so that the composition may be readily subdivided into equally effective dosage forms such as tablets, pills and capsules. This solid preformulation composition is then subdivided into unit dosage forms of the type described above containing from about 1 mg to about 15 mg, preferably about 10 mg, of macitentan or any amount or range therein. The tablets or pills of the composition can be coated or otherwise compounded to provide a dosage form affording the advantage of prolonged action. For example, the tablet or pill can comprise an inner dosage and an outer dosage component, the latter being in the form of an envelope over the former.

The liquid forms in which the compositions of the present disclosure may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The methods described herein may also be carried out using a pharmaceutical composition comprising macitentan and a pharmaceutically acceptable carrier. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, macitentan may be administered in a single daily dose, or the total daily dosage may be administered in divided doses of two, three or four times daily. In some embodiments, the macitentan is administered orally in the form of a tablet once daily.

For instance, for oral administration in the form of a tablet or capsule, the active drug component (e.g., macitentan) can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methylcellulose and the like. For parenteral administration, sterile suspensions and solutions are desired. Isotonic preparations which generally contain suitable preservatives are employed when intravenous administration is desired.

To prepare pharmaceutical compositions of the present disclosure, macitentan, as the active ingredient, may be intimately admixed with a pharmaceutical carrier according to conventional pharmaceutical compounding techniques, which carrier may take a wide variety of forms depending of the form of preparation desired for administration (e.g., oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain, the disclosure of which is hereby incorporated by reference.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc., the disclosures of which are hereby incorporated by reference.

The present disclosure also provides pharmaceutical products comprising a clinically proven safe and clinically proven effective amount of macitentan. Typically, the pharmaceutical product is a package or is packaged.

In some embodiments, the package includes a label. In certain embodiments, the label identifies the macitentan as a regulatory approved chemical entity. In other embodiments, the label provides instructions for use of macitentan for the treatment of portopulmonary hypertension. In further embodiments, the label provides data for reducing PVR relative to a placebo. In yet other embodiments, the label comprises data or instructions for improving liver transplant perioperative mortality risk category, improving MELD exception eligibility, or reducing the risk of removal from a liver transplant waiting list.

The following Example is provided to illustrate some of the concepts described within this disclosure. While the Example is considered to provide an embodiment, it should not be considered to limit the more general embodiments described herein. Further, in the following example, efforts have been made to ensure accuracy with respect to numbers used (e.g., amounts, temperature, etc.) but some experimental error and deviation should be accounted for.

Abbreviations

| | | | |
|---|---|---|---|
| 6MWD | 6-minute walk distance | 6MVVT | 6-minute walk test |
| ADR | Adverse drug reactions | AE | Adverse event |
| AESI | Adverse events of special | ALT | Alanine aminotransferase |
| | interest | | |
| ALP | Alkaline phosphatase | AST | Aspartate aminotransferase |
| ATC | Anatomic Therapeutic | AUCT | Area under the plasma |
| | Chemical | | concentration-time curve |
| | | | during one |
| | | | dosing interval |
| b.i.d. | Twice a day | BLQ | Below the limit of |
| | | | quantification |
| BMI | Body mass index | bpm | Beats per minute |
| CCB | Calcium channel blockers | CFR (US) | Code of Federal |
| Cl | Confidence interval | | Regulations |
| CL | Confidence limit | Cₘₐₓ | Maximum plasma |
| | | | concentration |
| CO | Cardiac output | CRO | Contract research |
| | | | organization |
| CSR | Clinical Study Report | CV | Coefficient of variation |
| CYP | Cytochrome P450 | DB | Double-blind |
| DBP | Diastolic blood pressure | eCRF | Electronic Case Report |
| | | | Form |
| EOMT | End of macitentan | EOS | End of Study |
| | treatment | | |
| EOT | End of Treatment | EOT-DB | End of Treatment |
| | | | (double-blind) |
| EOT-OL | End of Treatment | ERA | Endothelin receptor |
| | (open-label) | | antagonist |
| ERS | European Respiratory | EudraCT | European Union Drug |
| | Society | | Regulating Authorities |
| | | | Clinical Trials |
| FAS | Full Analysis Set | FC | Functional class |
| FDA (US) | Food and Drug | GCP | Good Clinical Practice |
| | Administration | | |
| HIV | Human immunodeficiency | HR | Heart rate |
| | virus | | |
| HVC | Hepatic vein | HVPG | Hepatic venous pressure |
| | catheterization | | gradient |
| ICF | Informed consent form | ICH | International Council for |
| | | | Harmonisation |
| IEC | Independent Ethics | ILSDRB | Independent Liver Safety |
| | Committee | | Data Review Board |
| IND | Investigational New Drug | IRB | Institutional Review Board |
| | (application) | | |
| i.v. | Intravenous | IXRS | Interactive voice/web |
| | | | recognition system |
| LFT | Liver function test | LOQ | Limit of quantification |
| LS | Least square | LVEDP | Left ventricular end diastolic |
| | | | pressure |
| MedDRA | Medical Dictionary for | MELD | Model for End-Stage |
| | Regulatory Activities | | Liver Disease |
| MHRA | Medicines and Healthcare | MMRM | Mixed-effect model |
| | products Regulatory | | repeated measure |
| | Agency | | |
| mPAP | Mean pulmonary | mRAP | Mean right atrial pressure |
| | arterial pressure | | |
| MTS | Macitentan Treated Set | NT-proBNP | N-terminal pro b-type |
| | | | natriuretic peptide |
| o.d. | Once daily | OL | Open-label |
| OLE | Open-label extension | OLT | Orthotopic liver |
| | | | transplantation |
| OR | Odds ratio | | |
| PAH | Pulmonary arterial | PAP | Pulmonary arterial pressure |
| | hypertension | | |
| PAWP | Pulmonary artery | PDE-5 | Phosphodiesterase type 5 |
| | wedge pressure | | |
| PD | Pharmacodynamic | PH | Pulmonary hypertension |
| PK | Pharmacokinetic | PKS | PK Set |
| PoPH | Portopulmonary | PPS | Per Protocol Analysis Set |
| | hypertension | | |
| PT | Preferred term | PVR | Pulmonary vascular |
| | | | resistance |
| RHC | Right heart catheterization | RND | Randomized Analysis Set |
| SAE | Serious adverse event | SAP | Statistical Analysis Plan |
| SAS ® | Statistical Analysis System | SBP | Systolic blood pressure |
| s.c. | Subcutaneous | SC | Steering Committee |
| SCR | Screened Analysis Set | SD | Standard deviation |
| SE | Standard error | sGC | Soluble guanylate cyclase |
| SI | International System | SMQ | Standardised MedDRA |
| | of Units | | Query |
| SOC | System organ class | SOP | Standard operating |
| | | | procedure |
| SS | Safety Set | SUSAR | Suspected unexpected |
| | | | serious adverse reaction |
| SVO2 | Mixed venous oxygen | TIPS | Transjugular intrahepatic |
| | saturation | | portosystemic shunt |
| tₘₐₓ | Time to reach maximum | TPR | Total pulmonary |
| | plasma concentration | | resistance |
| ULN | Upper limit of the normal | US | United States |
| | range | | |
| WHO | World Health | WHODRUG | WHO Drug Dictionary |
| | Organization | | |
| WU | Wood unit | LOCF | Last observation |
| | | | carried forward |
| BDI | borg dyspnea index | PT-INR | prothrombin time |
| | | | and international |
| | | | normalized ratio |
| EOT-OLE | end of treatment | UX-OLE | unscheduled visit |
| | (open-label extension) | | (open-label extension) |
| DDF | Denominator Degrees | LS Mean | Least Square Mean |
| | of Freedom | | |
| NDF | Numerator Degrees of | ANCOVA | analysis of covariance |
| | ANCOVA Freedom | | |
| ADaM | Analysis Data Model | CDISC | Clinical Data Interchange |
| | | | Standards Consortium |
| Cl | Confidence interval | ESC | European Society of |
| | | | Cardiology |
| IPAH | Idiopathic pulmonary | HCP | Healthcare professional |
| | arterial hypertension | | |
| LT | Liver transplant | LOCF | Last observation |
| | | | carried forward |
| PDE5I | Phosphodiesterase | SDTM | Study Data |
| | type 5 inhibitor | | Tabulation Model |

### Example 1: PORTICO Study

The study was conducted in seven countries including Brazil, Czech Republic, France, Germany, Spain, UK and US (52 sites were initiated, and patients were enrolled and randomized at 36 sites).

Study Objectives

Primary Objective

To evaluate the effect of macitentan on PVR as compared to placebo in patients with PoPH.

Secondary Objectives

To evaluate the effect of macitentan as compared to placebo on cardio-pulmonary hemodynamics, hepatic portal vein pressure, disease severity, and exercise capacity in patients with PoPH.

To evaluate the safety and tolerability of macitentan in patients with PoPH.

To evaluate the PK of macitentan and its active metabolite ACT-132577 in patients with PoPH (PK substudy).

Investigational Plan

A. Overall Study Design and Plan

A1. Main Study

This study was designed as a randomized, double-blind (DB), placebo-controlled, prospective, multicenter, parallel group Phase 4 study assessing the efficacy and safety of macitentan in PoPH. The study design included an OL period following DB End of Treatment (EOT), the details of which are included below. The study design required approximately 84 adult patients with PoPH to be randomized (1:1) to receive either macitentan 10 mg, or matching placebo, once daily (o.d.) orally. Subject allocation to treatment groups was stratified by background PAH specific therapy receipt (yes/no). Stratification by region of enrollment was also performed as the number of enrolled patients per center was expected to be low, and variation in total patient numbers on a country-to-country level was expected. Based on an attrition rate of 10%, it was expected to have 76 evaluable patients at Week 12/End of Treatment (double-blind) (EOT-DB). Subjects were required to have a confirmed diagnosis of PoPH with a PVR of ≥4 Wood units (WU; ≥320 dyn·s·cm⁻⁵) at enrollment but not have severe hepatic impairment (defined as Child-Pugh Class C or Model for End-Stage Liver Disease (MELD) score ≥19). Subjects were required to have 6-minute walk distance (6MWD) ≥50 m at enrollment but they could belong to any WHO FC. Patients were randomized at 36 sites in seven countries. The core study comprised the following periods:

Screening Period commenced from signature of the ICF and ended with patient randomization (up to 28 days after signed informed consent).

Double-Blind Treatment Period started immediately after randomization with the first dose of DB study treatment at the end of Visit 2/Day 1 and ended with EOT-DB on the day of the last dose of DB study treatment (scheduled Day 84, Week 12, or earlier in case of premature discontinuation of DB study treatment).

Open-Label Treatment Period started immediately after EOT-DB (for patients who reached Week 12 of DB study treatment) with the first dose of OL study treatment at the end of Visit 5 and ended with End of Treatment (open-label) (EOT-OL) on the day of the last dose of OL study treatment (scheduled Day 168, Week 24, or earlier in case of premature discontinuation of OL study treatment).

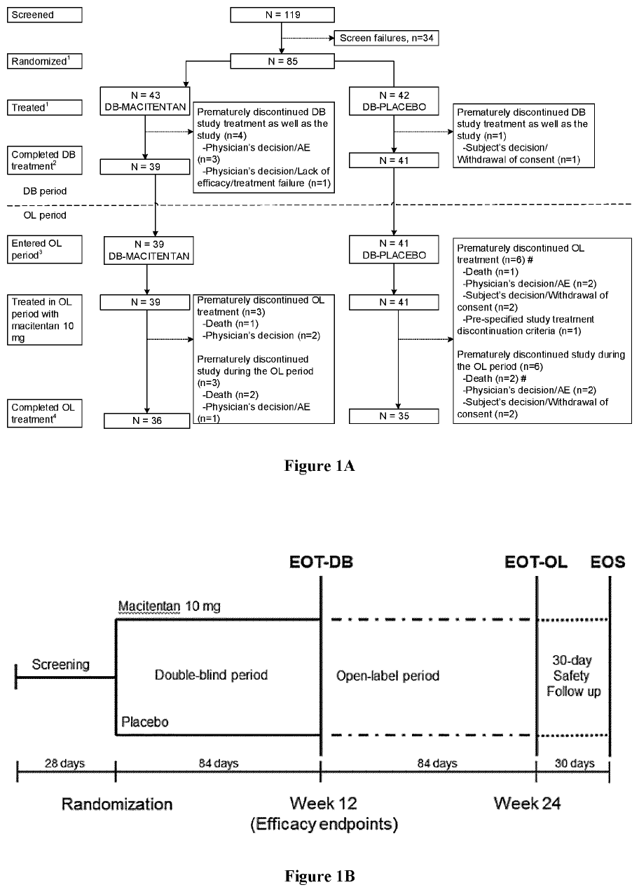

Safety Follow-up Period started immediately after the last dose of study treatment (DB study treatment or OL study treatment) and ended with the End of Study (EOS) 30 to 33 days after the last dose of either DB or OL study treatment. EOS was to have occurred in any patient discontinuing either DB or OL study treatment prematurely and who completed the Safety Follow-up Period. Subject participation in the study was a maximum of 33 weeks (up to 28 days Screening+24 weeks' treatment+30 days safety follow up). The overall study design is depicted in FIG. 1B.

A2. Open-Label Extension

The open-label extension (OLE) phase started immediately after EOT-OL for those patients randomized at French sites who completed the core phase of the study as scheduled and opted to continue receiving OL study treatment.

A3. Pharmacokinetic Substudy

It was planned to enroll at least 20 patients who were on steady-state treatment with macitentan (i.e., at least 4 weeks) into the PK substudy. The substudy was conducted in consenting patients from all centers across all regions who agreed to participate. The substudy consisted of the following periods:

Screening period: A Screening visit was scheduled within 28 days before enrollment into the PK substudy during which time the substudy was discussed with the patient including obtaining informed consent.

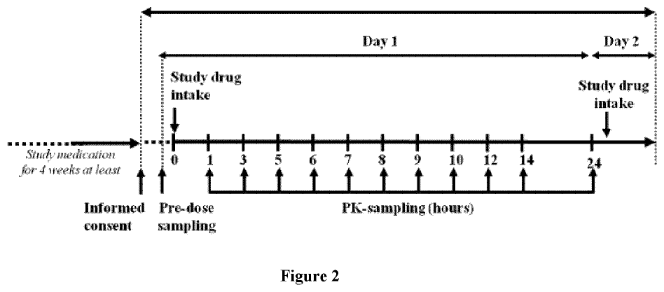

PK assessments period: PK assessments were to be performed after OL study treatment had been taken for at least 4 weeks. The substudy comprised two visits (Day 1: Enrollment and Day 2: Conclusion). See, FIG. 2. Subject eligibility was first checked on Day 1 of the PK substudy prior to the start of assessments.

B. Discussion of Overall Study Design

The 12-week DB treatment period was considered adequate to observe the anticipated treatment effect on the primary endpoint (change in PVR). Additionally, the risk of introducing bias from patients initiating add-on therapy was deemed relatively low in this timeframe, and the number of missing assessments due to premature study discontinuation was also expected to be minimized. The 12-week OL treatment period provided all patients with the opportunity to receive active treatment. It was possible for patients to receive iv. or subcutaneous (s.c.) prostanoid PAH-therapy at the investigator's discretion during the OL study period. A PK substudy was also conducted during the OL period in order to obtain information on the PK of macitentan in PoPH patients at steady state.

C. Study Population

C1. Selection of Study Population

This study enrolled adult male or female patients (≥18 years) with a confirmed diagnosis of symptomatic PoPH, a baseline PVR of ≥4 WU (≥320 dyn·s·cm⁻⁵) and who were capable of performing a 6-minute walk test (6MWT; with a 6MWD ≥50 m screening criterion). Patients could belong to any WHO FC. The study allowed patients to be enrolled who were either PAH-treatment naïve or were receiving background PDE-5 inhibitors, sGC stimulators, or inhaled prostanoid therapy. Patients with severe hepatic impairment, as defined by Child-Pugh class C liver disease or a MELD score 19 were excluded from the study. Females of childbearing potential were required to have a negative serum pregnancy test during screening and a negative urine pregnancy test prior to randomization on Day 1. Further stringent requirements of pregnancy testing and reliable methods of contraception were mandated for this enrolled subset during the study. All patients who reached Week 12 of the DB treatment period were considered eligible for entry into the OL period and received the first dose of OL macitentan treatment at the same visit. Of these, a total of 10 patients who had received OL macitentan treatment for at least 4 weeks and had consented to participate in the PK substudy procedures were enrolled into the PK substudy.

C2. Inclusion criteria

Main Study

Eligible patients were required to have fulfilled all of the following inclusion criteria. It was not permitted to waive any of the criteria for any patient:

1. Signed informed consent prior to any study-mandated procedure

2. Male or female ≥18 years of age with symptomatic PoPH:
Documented diagnosis of portal hypertension
PAH by RHC at screening: −mPAP ≥25 mmHg; −PAWP or left ventricular end diastolic pressure (LVEDP) ≤15 mmHg

3. PVR ≥4 WU or ≥320 dyn·s·cm⁻⁵ at screening (cardiac output measured by thermodilution technique only)

4. 6MWD ≥50 m at screening

5. Women of childbearing potential were required to:
Have a negative serum pregnancy test during screening and a negative urine pregnancy test prior to randomization on Day 1, and
Agree to use reliable methods of contraception from screening up to 30 days after study treatment discontinuation, and
Agree to perform monthly pregnancy tests up to 30 days after study treatment discontinuation.

PK Substudy
Signed ICF prior to initiation of any (PK) study-mandated procedure
Patients participating in the OL period of the main study and having received the OL study treatment for at least 4 weeks
Pre-dose PK blood sampling done prior to study medication on Day 1

C3. Exclusion Criteria

Main Study

Eligible patients were required to have had none of the following exclusion criteria. It was not permitted to waive any of the criteria for any patient:

1. PAH due to any other condition than portal hypertension

2. Severe hepatic impairment, as defined by Child-Pugh Class C liver disease or MELD score ≥19

3. Unstable liver disease (in the opinion of the investigator)

4. History of transjugular intrahepatic portosystemic shunt (TIPS) within 6 months prior to randomization

5. Documented severe obstructive or restrictive lung disease (in the opinion of the investigator)

6. Documented pulmonary veno-occlusive disease

7. SBP <90 mmHg at Screening

8. Body weight <40 kg at Screening

9. Patients undergoing dialysis

10. Initiation of diuretics or beta blockers within 1 week prior to baseline RHC or patients on oral diuretics or beta blockers in whom the dose has not been stable for at least 1 week prior to baseline RHC

11. Hemoglobin <100 g/L at Screening

12. Serum aspartate aminotransferase (AST) and/or alanine aminotransferase (ALT) ≥3×ULN at Screening

13. Bilirubin 3 mg/dL at Screening

14. Grades 2, 3, or 4 hepatic encephalopathy

15. History of liver transplantation

16. Documented hepatocellular carcinoma

17. Documented schistosomiasis infection

18. Gastrointestinal bleeding or esophageal variceal bleeding <3 months prior to randomization

19. Recently started (<3 months prior to randomization) or planned cardio-pulmonary rehabilitation program based on exercise

20. Treatment with calcium channel blockers (CCBs), an ERA, or i.v./s.c. or oral prostanoids within 3 months prior to randomization

21. Initiation, change in dose or discontinuation of PDE-5 inhibitor or sGC stimulator within 3 months prior to randomization

22. Treatment with interferon within 3 months prior to randomization

23. Treatment with any investigational drug within 3 months prior to randomization

24. Treatment with strong cytochrome P450 (CYP) 3A4 inducers (e.g., carbamazepine, rifampin, rifampicin, rifabutin, rifapentin, phenobarbital, phenytoin, and St. John's wort) within 4 weeks prior to randomization

25. Treatment with strong CYP3A4 inhibitors (e.g., ketoconazole, itraconazole, voriconazole, clarithromycin, telithromycin, nefazodone, ritonavir, boceprevir, telaprevir, saquinavir, lopinavir, fosamprenavir, darunavir, tipranavir, atazanavir, nelfinavir, amprenavir, indinavir, and paritaprevir) within 4 weeks prior to randomization

26. Known hypersensitivity to macitentan or its excipients or drugs of the same class

27. Pregnancy, breastfeeding, or intention to become pregnant during the study

28. Known concomitant life-threatening disease with a life expectancy of <6 months

29. Any known factor or disease that might interfere with treatment compliance, study conduct, or interpretation of results

30. Suspected or known current drug or alcohol abuse

PK Substudy
Clinical instability during the week prior to enrollment into the substudy
Ongoing renal failure or dialysis
Arm veins unsuitable for i.v. puncture

D. Study Treatment

Study treatment included DB as well as OL study treatment. DB study treatment comprised either investigational treatment (i.e., macitentan 10 mg) or matching placebo administered orally o.d.. OL study treatment consisted of OL macitentan 10 mg and was also administered orally o.d.

D1. Investigational treatment: The macitentan 10 mg tablet was administered orally o.d., in accordance with the Opsumit® prescribing information (Opsumit® SmPC), during the DB and OL treatment periods.

D2. Matching placebo: The matching placebo tablet was administered orally o.d. during the DB treatment period only.

D3. Study treatment administration

Main study: Eligible patients were required to take study treatment (during the DB and subsequent OL periods) o.d., with first administration taking place at site during randomization (Visit 2) and only after successful completion of the ICF procedure and all screening/randomization/eligibility assessments. At all subsequent visits (apart from PK substudy), study treatment was required to be taken after all study-mandated procedures had been performed. At home, patients were instructed to take one tablet (macitentan 10 mg or matching placebo) orally every morning irrespective of food intake. It was not permitted to take two tablets on the same day. In case of a missed dose, patients were instructed to take the next dose at the next scheduled time point (i.e., it was not permitted to take one tablet in the evening and the next dose the following morning).

PK substudy: On Day 1 of the PK substudy, the study treatment was required to be taken directly after the pre-dose blood sample. On Day 2 of the PK substudy, study treatment was required to be taken after the collection of the last PK blood sample (24 hours post Day 1 dose).

Rationale for dose selection: The dose selection for the study treatment was in accordance with the product labeling of the marketed drug, Opsumit®.

D4. Treatment Assignment

Eligible patients were randomized in a 1:1 ratio to either macitentan 10 mg or matching placebo. Treatment allocation was stratified based on receipt of background PAH therapy (Yes/No) and by region (Europe/North America/Latin America). All screened patients were assigned a study-specific patient number by the IXRS provider. Note: In case of re-screening, the original number was retained. This patient number was retained during the OL period of the study. After having confirmed the eligibility of the patient and prior to the start of study treatment, the investigator/delegate contacted the IXRS at randomization (Visit 2) to randomize the patient. The IXRS assigned a unique randomization number to the patient and assigned one unique treatment bottle number that matched the treatment arm assigned by the randomization list to the randomization number. The randomization list was generated using Statistical Analysis System (SAS®) version 9.3, and kept strictly confidential.

E Previous and Concomitant Therapy

E1. Allowed Concomitant Therapy
Oral PDE-5 inhibitor, inhaled prostacyclin analogs, or sGC stimulator were allowed if present for at least 3 months prior to randomization at a stable dose (which must remain unchanged during the DB treatment period unless the patient experiences worsening of PAH).
Treatment with oral diuretics was allowed if ongoing at a stable dose for at least 1 week prior to baseline RHC with the possibility for dose optimization during the treatment period.
Beta blockers were allowed if present for at least 1 week prior to baseline RHC and at a stable dose (which was required to remain unchanged during the study). If discontinuation of beta blockers occurred during the study the patient was required to complete the study as scheduled.
During the OL period, i.v. and s.c. prostanoid therapy (e.g., epoprostenol, treprostinil) was permitted at any time.
The following antiviral hepatitis C medications were permitted: simeprevir, sofosbuvir, daclatasvir, ombitasvir, dasabuvir, ledipasvir, etravirine, raltegravir, maraviroc, and ribavirin.

E2. Forbidden Concomitant Therapy
ERAs (e.g., bosentan, ambrisentan)
During the DB period, i.v./s.c. and oral prostanoid therapy (e.g., epoprostenol, treprostinil) were forbidden
CCBs.
Strong CYP3A4 inducers (e.g., carbamazepine, rifampin, rifampicin, rifabutin, rifapentin, phenobarbital, phenytoin, and St. John's wort)
Strong CYP3A4 inhibitors (e.g., ketoconazole, itraconazole, voriconazole, clarithromycin, telithromycin, nefazodone, ritonavir, lopinavir, fosamprenavir, darunavir, tipranavir, atazanavir, nelfinavir, amprenavir, indinavir, boceprevir, telaprevir, paritaprevir, and saquinavir)
Treatment with interferon
Any other investigational drug

F. Study Endpoints

For endpoints analyzed over the DB treatment period, the baseline was the value from the last non-missing assessment obtained prior to, i.e., before or on the day of, the start of study treatment (DB period). For endpoints analyzed over the macitentan treatment period, the macitentan baseline was defined as the last assessment prior to macitentan initiation.

F1. Efficacy Endpoints

Primary Efficacy Endpoint

The primary efficacy endpoint was:
Relative change from baseline to Week 12 in PVR, expressed as ratio of Week 12 to baseline PVR (Week 12 PVR divided by baseline PVR).

Secondary Efficacy Endpoints

The secondary efficacy endpoints were:
Change from Baseline to Week 12 in mean right atrial pressure (mRAP), mPAP, cardiac index, total pulmonary resistance (TPR), and mixed venous oxygen saturation (SVO₂), all measured at rest
Change from Baseline to Week 12 in WHO FC
Change from Baseline to Week 12 in 6MWD
Change from Baseline to Week 12 in N-terminal pro b-type natriuretic peptide (NT-proBNP)

Other Efficacy Endpoints

(i) Double-Blind Treatment Period
Change from Baseline to Week 12 in hepatic venous pressure gradient (HVPG)
Change from Baseline to Week 12 in HVPG (central review)
Change from Baseline to Week 12 in Borg dyspnea index

(ii) Macitentan Treatment Period

For patients who received macitentan in the DB or OL treatment period:
Change from macitentan Baseline to each (available) time point in WHO FC
Change from macitentan Baseline to each (available) time point in 6MWD
Change from macitentan Baseline to each (available) time point in NT-proBNP
Change from macitentan Baseline to each (available) time point in Borg dyspnea index

F2. Safety Endpoints

For the evaluation of treatment-emergent safety endpoints in the DB phase, the observation period for each individual subject started at the time of the first administration of macitentan or placebo and ended with the start of OL treatment or 30 days after DB treatment discontinuation (whichever occurred first). The safety variables listed below apply to the DB treatment-emergent period and the macitentan treatment-emergent period. The DB treatment-emergent period was defined as the period from the DB treatment start date up to the EOT-DB+30 days or to the EOT-DB for patients entering OL. The macitentan treatment-emergent period was defined as the period from the first intake of macitentan up to end of macitentan treatment (EOMT)+30 days. EOMT was defined as EOT-DB (for patient who received macitentan only in the DB period) or EOT-OL when applicable. For patients entering OLE, the EOMT is EOT-OL.
Treatment-emergent AEs
Treatment-emergent deaths
Treatment-emergent serious adverse events (SAEs)
AEs leading to premature discontinuation of study treatment
Occurrence of post-baseline LFT (ALT and/or AST) abnormalities, classified as:
   ≥3×ULN;
   ≥5×ULN;
   ≥8×ULN;
   ≥3×ULN and <5×ULN;
   >5×ULN and <8×ULN.
Occurrence of post-baseline ALT and/or AST abnormality 3×ULN and concomitant (i.e., at the same time) total bilirubin ≥2×ULN
Occurrence of post-baseline ALT and/or AST abnormality ≥3×ULN and concomitant (i.e., at the same time) total bilirubin ≥2×ULN and increased as compared to baseline
Occurrence of hemoglobin abnormalities, classified as:
   ≤80 g/L,
   >80 and ≤100 g/L,
   decrease from baseline ≥20 g/L and <50 g/L,
   decrease from baseline ≥50 g/L,
   <100 g/L and concurrent (i.e., at the same time) decrease from baseline ≥20 g/L.
Other treatment-emergent marked laboratory abnormalities
Change from baseline/macitentan baseline to EOT-DB/EOMT in vital signs
Change from baseline/macitentan baseline to EOT-DB/EOMT in laboratory variables
Change from baseline/macitentan baseline to EOT-DB/EOMT in Child-Pugh classification
Change from baseline/macitentan baseline to EOT-DB/EOMT in MELD score

F3. Pharmacokinetic Endpoints for Macitentan and its Active Metabolite ACT-132577
The area under the plasma concentration-time curve during one dosing interval (AUCₜ).
Maximum plasma concentration (Cₘₐₓ) during a dosing interval.
The time to reach maximum plasma concentration (tₘₐₓ) during a dosing interval.

F4. Appropriateness of Endpoints

The primary endpoint is the relative change from baseline to Week 12 in PVR in enrolled patients with PoPH. Hemodynamic measures provide a robust indicator of right ventricular function and prognosis. In severe PoPH, high PVR is considered to be a contraindication for liver transplant.

Exercise capacity is one of the most important prognostic indicators in PAH. It correlates well with peak aerobic capacity and, as such, has received acceptance by regulatory agencies. The short-term improvement in the 6MWD has been the most frequently used primary endpoint in the pivotal studies for the registration of PAH drugs. In addition, the guidelines of The American Thoracic Society recommend assessing patients' dyspnea after performing the 6MWT using the Borg dyspnea index. Given the large proportion of patients in WHO FC II and the extent of PAH therapy at baseline, achieving improvements in measures of functional capacity is likely to be challenging. Among patients originally randomised to macitentan and followed through to the open-label period of the study, there was 20 m increase in walk distance from baseline to Week 24, although with the caveat that from Week 12 onward the patients were no longer blinded to their treatment. Thus, this tool provides relevant information on how a patient perceived his/her dyspnea after exercise.

NT-proBNP levels correlate with myocardial dysfunction and functions as a strong predictor of prognosis in patients with PH. However, NT-proBNP is reported to be lower in obese patients and therefore may not be a reliable prognostic marker in obese patients. In patients without a TIPS, HVPG reflects the vascular resistance in the liver. An improvement in liver blood flow may translate into a lower HVPG.

F5. Study Assessments

When applicable, study assessments were required to be performed in the following order:
Blood sampling
Physical examination
WHO FC
6MVWT
Borg dyspnea index
Right heart/hepatic vein catheterization at rest

Baseline Assessments

(i) Visit 1/Screening (or Visit 1a/Re-Screening, if Applicable)

The following were recorded at Visit 1/Screening.
Date of informed consent, baseline demographics (sex, age, race, ethnicity, body weight, height) as well as a reason for why a female patient was not considered to be of childbearing potential (as applicable)
Complete, clinically relevant medical history (previous and ongoing at randomization) and the following liver disease characteristics:
Date and mode of portal hypertension diagnosis (liver biopsy, hepatic vein catheterization, ultrasound, presence of esophageal varices, ascites, history of TIPS, other)
Cause of portal hypertension: Intrahepatic (autoimmune hepatitis, alcohol cirrhosis, hepatitis B cirrhosis, hepatitis C cirrhosis, nonalcoholic steatohepatitis, primary biliary cirrhosis); pre-hepatic (portal vein thrombosis/clots, congenital); post-hepatic (hepatic vein thrombosis, inferior vena cava thrombosis); other
Child-Pugh class and/or MELD score were to be assessed and documented
PAH disease characteristics such as 6MWD, Borg dyspnea index, WHO FC, as well as vital signs, body mass index (BMI), and ongoing medications
RHC and hepatic vein catheterization (HVC) data. HVC was not a required procedure for study entry but was required to be collected where possible
Reason for Screen failure, if applicable
Abnormal findings from physical examination were to be recorded as AEs
Laboratory tests (to assess eligibility (Table 1)) and serum pregnancy testing
Any SAEs occurring during screening relating to study-related procedures

(ii) Visit 2/Randomization

The following were recorded at Visit 2/Randomization:
6MWD, Borg dyspnea index, WHO FC, and ongoing medications
NT-proBNP
Any AEs and SAEs occurring during Randomization
Serum laboratory and pregnancy testing (including urine dipstick testing)

Efficacy Assessments

(i) Hemodynamic measurements—right heart catheterization: Invasive cardiac hemodynamic variables were measured according to the RHC guidelines at Baseline (Visit 1/Screening) and Week 12, or at permanent discontinuation of DB study treatment if prior to Week 12 (at the investigator's discretion). The thermodilution technique was used to measure cardiac output (CO). For each patient, a maximum of two RHC procedures were performed during the study. The following variables were measured: Heart rate (HR), PAWP or LVEDP (if PAWP is not available), mRAP, systolic/diastolic/mean pulmonary arterial pressure (PAP), CO, SVO₂. PVR was calculated at Visit 1/Screening. The following hemodynamic variables were calculated for analysis purposes: PVR, TPR, cardiac index.

(ii) Hemodynamic measurements—hepatic vein catheterization: HVPG, the difference between portal pressure and hepatic pressure, was measured by HVC at Baseline (Visit 1/Screening) and Week 12, or at permanent discontinuation of DB study treatment if prior to Week 12 (at the investigator's discretion). Where possible, HVPG was measured at the same time as RHC. The second measurement during the study was required to be performed within 1 week prior to EOT-DB if it was not possible to perform the HVC on the same day as the RHC. HVPG measurements were performed after at least 4 hours of fasting, at rest, in the supine position, under local anesthesia and mild sedation. A catheter introducer was placed into the right jugular vein (or femoral) using the Seldinger technique. It was used to advance a balloon-tipped catheter into the main right hepatic vein for repeated measurements of wedged (occluded) and free hepatic venous pressures. Intravascular pressures were measured using highly sensitive pressure transducers, calibrated before each measurement. HVPG was calculated as the difference between wedged and free hepatic venous pressures. Measurements were performed at least as duplicates and repeated until, ideally, the two consecutive reliable measurements did not differ by more than 1 mmHg. The mean of these two measurements was considered final.

(iii) WHO functional class: WHO FC was evaluated at Screening, Baseline (Visit 2/Randomization), and then every visit until EOT-DB or EOT-OL, whichever came last.

(iv) 6-minute walk test, including Borg dyspnea index: The 6MWT was performed at Screening, Baseline (Visit 2/Randomization), and then every Visit until EOT-DB or EOT-OL, whichever came last. It is a non-encouraged test that measures the distance walked by the patient in six minutes. It is important that for each individual patient the 6MWT was conducted under the same conditions throughout the study (e.g., same location, same tester, same time of day, with or without nasal oxygen therapy and where applicable with same flow rate as Baseline). The Borg dyspnea index was evaluated after each 6MWT. It rates dyspnea on a scale from 0 to 10.

(v) Serum NT-proBNP: A blood sample was drawn at Baseline (Visit 2/Randomization), EOT-DB, and/or EOT-OL (whichever came last) for the analysis of serum NT-proBNP. Serum NT-proBNP samples were shipped to the central laboratory on dry ice as soon as possible after the blood sample had been drawn or stored frozen at −20° C. (−4° F.)±2° C. (±3.6° F.) until the shipment.

Safety Assessments

(i) Adverse events: All AEs occurring after study start (i.e., signing of informed consent) and up to 30 days after study treatment discontinuation were required to be recorded. AEs still ongoing more than 30 days after study treatment discontinuation were required to be followed up until they were no longer considered clinically relevant.

(ii) Serious adverse events, including associated with study design or protocol-mandated procedures: During the screening period, all SAEs occurring after study start (i.e., signing of informed consent) were required to be recorded (whether considered associated or not associated to study design or study-mandated procedures). All SAEs, regardless of investigator-attributed causal relationship, occurring during the treatment period up to the end of the 30-day follow up period, were required to be reported. SAEs still ongoing at the EOS visit were required to be followed up until resolution or stabilization, or until the event outcome was provided, e.g., death. New SAEs occurring after the 30-day follow-up period were required to be reported within 24 hours.

(iii) Child-Pugh and/or MELD score: Assessment of liver disease severity using the Child-Pugh classification and/or MELD Score was performed at Screening/Visit 1, EOT-DB, and EOT-OL and entered into the eCRF.

Pharmacokinetic Assessments

(i) Timing for sampling: For the PK assessment, blood samples were drawn at the following time points:
PK substudy Day 1: Immediately before study treatment administration in the morning (pre-dose) and 1 h, 3 h, 5 h, 6 h, 7 h, 8 h, 9 h, 10 h, 12 h, and 14 h post-dose.
PK substudy Day 2: 24 h post-dose.

In order to ensure timely blood sampling, patients were offered an overnight stay at the clinic or for sampling to be performed at a location close to the patient's home.

(ii) Procedures for sampling: The total blood volume collected for PK assessments from an individual patient was approximately 12×2 mL=24 mL.

(iii) Bioanalysis: A validated liquid chromatography coupled to mass spectrometry method was used for determining the concentrations of macitentan and its metabolite ACT-132577 in plasma. The limit of quantification (LOQ) for both analytes was 1 ng/mL.

F6. Schedule of Visits and Assessments

Subjects were required to have given written informed consent before participating in any study procedures. An overview of the chronological sequence of assessments for the core study is provided in Tables 1 and 2 for the OLE.

TABLE 1
Schedule of visits and assessments

| | | | Double-Blind | | | Open-Label | | | |
|---|---|---|---|---|---|---|---|---|---|
| | | | Treatment | | | Treatment | | | |
| | | | 12 weeks | | | 12 weeks | | | |
| | | | | 3/4 | 5 | 6/7 | 8 | U1, U2 . . . | Follow-up |
| | | | | Visits | EOT- | Visits | EOT- | Unscheduled | 30 days |
| | Name | Screening | | 3/4 | DB 1 | 6/7 | OL¹ | visit² | EOS³ |
| | Duration | 28 days | 2 | Weeks | Week | Weeks | Week | Any day | EOT- |
| | Number | 1 | Random- | 4/8 | 12 | 16/20 | 24 | between Day | DB/OL + |
| Periods | Name | Screening | ization | (±4 | (±4 | (±4 | (±4 | 1 and Week | 30-33 |
| VISITS | Time | Day −28 | Day 1 | days) | days) | days) | days) | 24 | days |

| | | | | | | | | |
|---|---|---|---|---|---|---|---|---|
| Informed consent | X | | | | | | | |
| Medical history | X | | | | | | | |
| Demographics | X | | | | | | | |
| Concomitant therapy | X | X | X | X | X | X | X | |
| Physical examinatione⁴ | X | X | X | X | X | X | X | |
| Vital signs | X | | | X | | X | X | |
| Height⁵ and weight | X | | | X | | X | | |
| Laboratory tests⁶ | X⁷ | X | X | X | X | X | X | |
| Serum pregnancy test | X | X⁸ | X | X | X | X | X | X¹⁵ |
| Child-Pugh | X | | | X | | X | | |
| assessment and/or | | | | | | | | |
| MELD Score | | | | | | | | |
| Right heart | X | | | X | | | | |
| catheterization⁹ | | | | | | | | |
| Hepatic vein | X | | | X¹¹ | | | | |
| catheterization¹⁰ | | | | | | | | |
| 6MWT & BDI | X | X | X | X | X | X | X | |
| WHO functional class | X | X | X | X | X | X | X | |
| NT-proBNP | | X | | X | | X | X | |
| PK substudy | | | | | X¹² | | | |
| Study drug | | X | X | X | X | X | | |
| dispensing/return¹³ | | | | | | | | |
| AEs¹⁴ | X | X | X | X | X | X | X | X |
| SAEs¹⁴ | X | X | X | X | X | X | X | X |

¹If patient did not reach Week 12 (Visit 5) or did not enter the open-label phase, the EOT-DB visit was to be performed including RHC, followed by EOS after 30 days. If a patient entered the open-label phase but did not reach Week 24 (Visit 8), the EOT-OL visit was to be performed but without RHC, followed by EOS after 30 days.
²Unscheduled visits could be performed at any time during the study and included all or some of the indicated assessments, based on the judgment of the investigator.
³Visit could be performed by telephone.
⁴Data not collected in the eCRF; abnormal findings recorded as AEs.
⁵Height recorded at Screening only.
⁶Includes hematology, general blood chemistry, PT-INR performed monthly during study conduct.
⁷Eligibility assessed using Screening laboratory data only.
⁸Urine dipstick pregnancy test performed in addition to serum.
⁹Cardiac output measured using thermodilution technique only.
¹⁰HVC was not required at sites that did not perform the procedure routinely.
¹¹Was to be performed on or within 7 days prior to EOT-DB (if previously performed at Baseline).
¹²Separate PK substudy informed consent form had to be completed prior to any substudy procedure. Could be performed at any time point on or between Visits 6 and 7.
¹³Scheduled study treatment dispensing/return procedures could be adapted according to the site practice.
¹⁴All AEs and SAEs that occurred after signing the informed consent form and up to 30 days after study treatment had to be reported.
¹⁵Urine dipstick test.

TABLE 2
Open-label extension visit and assessment schedule

| | | OPEN-LABEL EXTENSION | | | Unscheduled | FOLLOW-UP |
|---|---|---|---|---|---|---|
| | | Variable | | | visit² UX-OLE | 30 days |
| | | | Visits 10, | | Any day | EOS3 |
| | Name | | 11,12 etc. | | between | EOT- |
| | Duration | Visit 9 (OLE | Every 12 | | enrolment | OLE + |
| PHASE | Name | Enrolment)¹ | weeks | EOT- | visit and | 30-33 |
| VISITS | Time | Week 24 | (±7 days) | OLE | EOT-OLE | days |

| | | | | | |
|---|---|---|---|---|---|
| Concomitant therapy | X | X | X | X | |
| Physical examination⁴ | X | X | X | X | |
| Laboratory tests⁵ | | Monthly | X | X | |
| | | (±4 days) | | | |
| Serum pregnancy test | | Monthly | X | X | X⁵ |
| | | (±4 days) | | | |
| PK substudy | | X⁹ | | | |
| Study drug | X | X | X | | |
| dispensing/return⁶ | | | | | |
| AEs⁷ | X | X | X | X | X |
| SAEs⁷ | X | X | X | X | X |

¹Visit 9 (OLE enrollment) occurred on the same day as EOT-OL of the core phase of the study.
²Unscheduled visits could be performed at any time during the study and could include all or some of the indicated assessments, based on the judgment of the investigator.
³Visit could be performed by telephone.
⁴Data not collected in the eCRF; abnormal findings recorded as AEs.
⁵Includes liver function tests, hemoglobin. Performed monthly during OLE conduct.
⁶Scheduled study treatment dispensing/return procedures could be adapted according to the site practice.
⁷All AEs and SAEs that occurred after signing the informed consent form and up to 30 days after study treatment discontinuation had to be reported.
⁸Urine dipstick test.
⁹Separate PK substudy informed consent form was completed prior to any substudy procedure. Could be performed at any time point on or between Visit 10 and EOT-OLE.

G1. Analysis Sets

The following analysis sets were defined for this study.

(i) Screened Analysis Set: The Screened Analysis Set (SCR) includes all patients who were screened and received a patient number.

(ii) Randomized Analysis Set: The Randomized Analysis Set (RN0) includes all patients from the SCR who were randomized.

(iii) Full Analysis Set: The Full Analysis Set (FAS) includes all randomized patients who received at least one dose of study treatment in the DB treatment period and have a baseline value for the primary endpoint (PVR). In order to adhere to the intention-to-treat principle as much as possible, patients were evaluated according to the study treatment to which they were assigned to (which may be different from the study treatment that they received).

(iv) Per Protocol Analysis Set: The Per-Protocol Analysis Set (PPS) comprises all patients included in the FAS without major protocol deviations that might have affected the main analysis of the primary efficacy variable.

(v) Safety Set: The Safety Set (SS) includes all patients who received at least one dose of study treatment in the DB treatment period, based on the actual treatment received.

(vi) Other analysis sets: The Macitentan Treated Set (MTS) consists of all patients who received at least one dose of macitentan in the DB or OL treatment period. The MTS is based on treatment actually received. The PK Set (PKS) comprises all patients from the FAS, for whom a PK blood sample at trough on Day 1 was taken and who did not deviate from the protocol in a way that might affect the evaluation of the PK endpoints.

(vii) Usage of the analysis sets

Primary efficacy analysis was performed on the FAS and a sensitivity analysis was performed on the PPS.

Secondary efficacy analyses were performed on the FAS.

Safety analyses related to the DB treatment period were performed on the SS.

Summaries of efficacy and safety data obtained in all patients who received macitentan (regardless of the treatment period, except the OLE) were performed on the MTS.

Summaries of baseline characteristics were performed on the FAS, PPS and MTS.

Patient listings are based on the SCR, except listings of randomization data and protocol deviations, which are based on the RND, and listings of exposure data, which are based on the SS.

PK substudy analysis was performed on the PKS.

G2. Variables

(i) Primary efficacy variable

The primary efficacy endpoint is:
Relative change from baseline to Week 12 in PVR, expressed as ratio of Week 12 to baseline PVR (Week 12 PVR divided by baseline PVR).
Calculated PVR was used for the efficacy analyses.

(ii) Secondary efficacy variables

Secondary efficacy variables are changes from baseline to Week 12 in:
mRAP (mmHg)
mPAP (mmHg)
Cardiac index (L/min/m²)
TPR (dyn·s·cm⁻⁵)
SVO₂ (%)
WHO FC (I-IV)
6MWD (m)
NT-proBNP (ng/L)

Calculated cardiac index and TPR were used for the efficacy analyses. Other RHC variables (HR, systolic PAP, diastolic PAP, CO, and PAWP/LVEDP) are listed only.

(iii) Other Efficacy Variables

Double-blind treatment period: For all patients, other efficacy variables are changes from baseline to Week 12 in:
HVPG (mmHg)
HVPG (mmHg; central review)
Borg dyspnea index

Macitentan treatment period: For patients who received macitentan in the DB or OL treatment period, other efficacy variables are changes from macitentan baseline to each available time point for the following:
WHO FC
6MWD (m)
NT-proBNP (pg/mL)
Borg dyspnea index

(iv) Safety Variables

The safety variables were analyzed over the DB treatment-emergent and macitentan treatment-emergent periods.

Adverse events, including deaths during the DB treatment period and the macitentan treatment period.

In addition, the following adverse events of special interest (AESIs) were analyzed:
“Edema and fluid retention”

Any treatment-emergent AE with PT listed in the Standardised MedDRA Query (SMQ) “Haemodynamic oedema, effusions and fluid overload (SMQ)” or with PT equal to “Pulmonary congestion” defined in the latest available MedDRA version, with the exception of PTs containing “site”.
“Anemia”

Any treatment-emergent AE with a PT within the SMQs “Haematopoietic erythropenia” OR “Haematopoietic cytopenias affecting more than one type of blood cell (SMQ)” (with the exception of two unspecific PTs: “blood disorder”, “blood count abnormal”) OR an event with any MedDRA PT containing the text “anaemia”.
“Drug related hepatic disorders”

Any treatment-emergent AE with a PT within the SMQ “Drug related hepatic disorders”.

Treatment-emergent liver test and hemoglobin abnormalities are those that occurred during the DB treatment-emergent (SS) and macitentan treatment-emergent (MTS) periods that were not present at baseline.

For post-baseline liver test abnormalities, the following events are considered:
ALT and/or AST ≥3×ULN,
ALT and/or AST ≥5×ULN,
ALT and/or AST ≥8×ULN,
ALT and/or AST ≥3×ULN and <5×ULN,
ALT and/or AST ≥5×ULN and <8×ULN,
ALT and/or AST ≥3×ULN and concomitant (i.e., at the same time) total bilirubin ≥2×ULN.
ALT and/or AST 3×ULN and concomitant (i.e., at the same time) total bilirubin ≥2×ULN and increased as compared to baseline

The highest ALT or AST value at any post-baseline time point of assessment for DB treatment-emergent (SS) and macitentan treatment-emergent (MTS) periods was considered in the evaluation of incidences.

For the occurrence of post-baseline hemoglobin abnormalities, the following events were considered in the evaluation of incidences:
Hemoglobin ≤80 g/L,
Hemoglobin >80 and ≥100 g/L,
Hemoglobin decrease from baseline ≥20 g/L and <50 g/L,
Hemoglobin decrease from baseline ≥50 g/L,
Hemoglobin <100 g/L and concurrent (i.e., at the same time) decrease from baseline ≥20 g/L.

The lowest hemoglobin value at any post-baseline time point of assessment for DB treatment-emergent (SS) and macitentan treatment-emergent (MTS) periods was considered in the evaluation of incidences.

G3. Description of Statistical Analyses

(i) Overall Testing Strategy

The protocol overall type I error was set to α=0.025 (one-sided), which corresponds to a 2-sided α=0.05 (which was used for all primary and secondary efficacy analyses) in the sample size calculations. The type II error was set to 0.10 and the power to 90%. No adjustment was made for multiplicity for secondary endpoints, therefore all corresponding p-values provided are of an exploratory nature.

(ii) Analysis of the Primary Efficacy Variable(s)

Main Analysis

The primary analysis was performed on the FAS and using calculated PVR. PVR was summarized by time point and treatment group using descriptive statistics. Ratio of Week 12 to baseline was summarized using geometric means and coefficients of variation (CVs). The ratio of Week 12 to baseline PVR was log-transformed (base e) and analyzed using ANCOVA with factors for treatment group (macitentan versus placebo), background PAH-specific therapy at baseline (Yes/No as per IXRS, randomization stratification factor), region (Europe/North America/Latin America as per IXRS, randomization stratification factor) and a covariate for baseline log-transformed PVR. The treatment group difference (on log scale) and its 95% confidence interval (2-sided) were estimated based on the model. The geometric mean ratio (macitentan versus placebo) and its 95% confidence interval (2-sided) were obtained by exponentiation. The null hypothesis was to be rejected if the entire 95% confidence interval (2-sided) did not include 1.0. The log transformation for PVR is justified by the fact that ratios versus baseline follow a normal distribution more closely after a log transformation. In addition, mean absolute changes from baseline on log scale can be translated into (geometric) mean ratios by exponentiation.

Supportive/Sensitivity Analyses

A sensitivity analysis was performed applying main ANCOVA analysis without the stratification factors (background PAH-specific therapy at baseline and region). Another sensitivity analysis was performed applying main ANCOVA analysis on change from baseline to Week 12 in PVR (no log transformation). Another sensitivity analysis was performed on the FAS where, for patients with a post-baseline PVR measurement obtained before Week 12 (outside window), the following imputations were used for the log-transformed PVR:

$$Log [ \frac{P V {R}_{Week 12}}{P V {R}_{Baseline}} ] = Log [ \frac{P V {R}_{At Week t}}{P V {R}_{Baseline}} ] + \frac{( 84 - t )}{84 \times slope}$$

wherein:
t is the treatment day of the measurement of post-baseline PVR
slope is the expected change from baseline in log PVR based on the main analysis model, restricted to patients who had their post-baseline PVR measurement at Week 12 (FAS without imputation) in the corresponding treatment group.

If no post-baseline assessment was available, then Log [PVRWeek 12/PVRBaseline] was imputed by “slope” in the corresponding treatment group. Other sensitivity analyses were performed on the PPS and on observed data (FAS without imputation).

Subgroup Definitions and Analyses

In order to verify consistency of results, subgroup analyses were performed (using the FAS) applying main analysis for all subgroups shown below. The PAH background therapy at baseline and the region subgroups were defined in the protocol).

1. All patients with versus without PAH background therapy at baseline (as per IXRS, randomization stratification factor)

2. All patients by region: Europe/North America/Latin America (as per IXRS, randomization stratification factor)

3. All patients by baseline WHO FC: I-II versus III-IV

4. All patients by gender: male/female

5. All patients by esophageal varices at baseline: yes/no

6. All patients by baseline Child Pugh: A/B/C

7. All patients by age: <65 versus ≥65

Interaction tests for treatment and subgroup variable were performed by adding the subgroup variable and treatment-by-subgroup variable interaction terms to the statistical main model (i.e., one model per subgroup variable). Interaction tests were performed at the 0.05 level. The ratio of geometric mean treatment effect estimates along with their corresponding 95% 2-sided confidence limits (CLs) per subgroup are presented in a forest plot together with p-values of interaction tests.

(ii) Analysis of the Secondary Efficacy Variable(s)

Hypothesis and statistical model: Secondary efficacy analyses were performed on the FAS at α=0.05 (two-sided). No correction for multiple testing was applied for these analyses.

Handling of missing data: For change from baseline to Weeks 4, 8 and 12 in 6MWD and WHO FC, the last available value (including baseline) was carried forward unless the patient died before, or on Day 42, 70, 98 (the upper limit treatment days of Weeks 4, 8 and 12 windows, respectively), in which case the following rules considering worst case were used to replace all the missing values from the week after death up to Week 12:
For 6MWD, worst fixed value was set to 0,
For WHO FC, worst fixed value was set to class IV,
For change from baseline to Week 12 in NT-proBNP, the same imputation rule as for PVR was applied as described herein.

For change from baseline to Week 12 in mRAP, mPAP, cardiac index, TPR and SVO₂: in patients with a post-baseline measurement obtained before Week 12, the post-baseline measurement was carried forward. This imputation was performed unless one of the following occurred:
If a subject died without a Week 12 value then the Week 12 value was imputed using the largest ratio of baseline mRAP, mPAP, TPR (the lowest for cardiac index, SVO₂) at Week 12 in all subjects in the same treatment group and analysis set, such that this value was multiplied by the subject's baseline value to obtain the imputed Week 12 value.
If the subject was alive and did not have a post-baseline value then the median of the ratio of baseline value at Week 12 from all subjects in the same treatment group and analysis set was used to impute the Week 12 value, such that this value was multiplied by the subject's baseline value to obtain the imputed Week 12 value.

Only observed values were used to calculate the imputed values. Patients with a missing baseline value for a given variable were not included in the analysis.

Statistical analysis: The change from baseline to Week 12 in mRAP, mPAP, cardiac index, TPR and SVO₂ were analyzed on the FAS (with imputation) using an ANCOVA with factors for treatment group, background PAH-specific therapy at baseline (Yes/No as per IXRS, randomization stratification factor), region (Europe/North America/Latin America as per IXRS, randomization stratification factor) and a covariate for baseline corresponding variable. Other RHC variables are listed only. WHO FC is summarized on the FAS (with imputation) by time point and treatment group using frequency tables. Changes from baseline in WHO FC were dichotomized as worsening (i.e., change >0) versus no change or improvement (i.e., change ≤0). Worsening was analyzed using a logistic regression model (exact) with covariates for treatment group, background PAH-specific therapy at baseline (Yes/No as per IXRS, randomization stratification factor) and region (Europe/North America/Latin America as per IXRS, randomization stratification factor). 6MWD is summarized on the FAS (with imputation) by time point and treatment group using descriptive statistics. Change from baseline to each time point is summarized similarly. Change from baseline in 6MWD (up to Week 12) was analyzed using a mixed-effect model repeated measure (MMRM) with factors for treatment group, time point, treatment by time interaction, background PAH-specific therapy at baseline (Yes/No as per IXRS, randomization stratification factor) and region (Europe/North America/Latin America as per IXRS, randomization stratification factor), and covariates for baseline 6MWD and WHO FC. An unstructured covariance matrix was used to account for the correlation between repeated measurements from the same patient. NT-proBNP is summarized on the FAS (with imputation) by time point and treatment group using descriptive statistics as well as geometric means and CVs. The ratio of Week 12 to baseline NT-proBNP is summarized similarly. The ratio versus baseline in NT-proBNP was log-transformed and analyzed using an ANCOVA with covariates for treatment group, background PAH-specific therapy at baseline (Yes/No as per IXRS, randomization stratification factor), region (Europe/North America/Latin America as per IXRS, randomization stratification factor) and baseline log NT-proBNP.

Supportive/sensitivity analyses: The main MMRM model analysis for 6MWD was also performed without stratification factors. In addition, change from baseline to Week 12 in 6MWD was analyzed using an ANCOVA with factors for treatment group, background PAH-specific therapy at baseline (Yes/No as per IXRS, randomization stratification factor), region (Europe/North America/Latin America as per IXRS, randomization stratification factor) and a covariate for baseline 6MWD. Supportive analyses were also performed for secondary endpoints for the FAS without imputation (observed cases). In addition, a scatter plot of change in PVR from baseline to Week 12 versus change in 6MWD from baseline to Week 12 is provided.

Subgroup analyses: For 6MWD main and supportive analyses, forest plots are provided for subgroup analyses (same variables as primary endpoint).

(iii) Analysis of the Other Efficacy Variables

Double-blind treatment period: Changes from baseline to Week 12 in HVPG/HVPG (central review) and Borg dyspnea index (related to the DB treatment period) are summarized by treatment group in the FAS using descriptive statistics.

Macitentan treatment period: Changes from macitentan baseline to each time point in WHO FC, 6MWD, NT-pro BNP and Borg dyspnea index (related to the macitentan treatment period) are summarized for the MTS using descriptive statistics.

(iv) Analysis of Pharmacokinetic Variables

The measured individual plasma concentrations of macitentan and its metabolite ACT-132577 were used to directly obtain Cₘₐₓ and tₘₐₓ. AUC_{T} was calculated according to the linear trapezoidal rule using the measured concentration-time values above the LOQ during one dosing interval. The PK variables were calculated on the basis of the real (actual) blood sampling time points. For mean value calculations, all values below the LOQ (below the limit of quantification (BLQ) values) were set to zero if ≤50% of the values at a given time point were BLQ. If >50% of the values at a given time point were BLQ, no mean value was calculated. Mean concentration-time profiles were generated using these criteria. The individual and mean plasma concentration-time profiles were plotted on a linear scale. The derived PK variables for macitentan and ACT-132577 (AUC_{T}, Cₘₐₓ, tₘₐₓ) are listed by patient. Plasma concentrations per time point were summarized using arithmetic mean, minimum, median, maximum, standard deviation (SD), standard error (SE), and two-sided 95% confidence interval of the mean. AUCₜ, Cₘₐₓ, tₘₐₓ* were summarized with arithmetic mean, geometric mean, minimum, median, maximum, SD, SE, CVb in %, and 95% confidence interval of the arithmetic and geometric means. (*For tₘₐₓ the geometric mean and its 95% confidence interval were not calculated).

Study Subjects

A. Disposition of Patients

Patient disposition is shown in FIG. 1. A total of 119 patients at 39 sites in seven countries were screened. Of these, 85 patients at 36 sites were randomized (43 to macitentan 10 mg o.d. and 42 to matching placebo) and received study treatment. Overall, 80 patients (39 and 41 patients in the macitentan and placebo groups, respectively) completed the 12-week DB treatment period. All 80 patients who completed the DB treatment period entered the 12-week OL treatment period and received macitentan 10 mg o.d. Of these, 71 (36 DB macitentan and 35 DB placebo) completed the OL treatment period. See, FIG. 1. Of those who completed the OL treatment period, 33 patients entered the OLE and continued receiving macitentan 10 mg o.d. At the time of database lock of the core study, 29 patients were ongoing in the OLE period.

B. Analysis Sets

An overview of analysis sets is provided in Table 3. All 85 patients randomized into the study were included in the FAS. The SS was identical to the FAS. The proportion of patients included in the PPS was 83.7% in the macitentan group and 88.1% in the placebo group. A total of 12 patients (macitentan: 7, placebo: 5) were excluded from the PPS. The most frequently reported reason for exclusion from the PPS was receipt of prohibited concomitant medication or dose change/discontinuation of concomitant medication during the DB treatment period that might have had an effect on the primary endpoint (macitentan: 3, placebo: 2). Other reasons included inclusion criterion on PVR not met, treatment with CCB, ERA, iv./s.c., or oral prostanoids in the 3-month period prior to randomization, and stratification not performed as per protocol. The PKS included 10 patients. See, Table 3. A total of 84 patients received at least one dose of macitentan in the DB or OL treatment period and were included in the MTS.

TABLE 3
Overview of analysis sets

| | Macitentan | | |
|---|---|---|---|
| | 10 mg | Placebo | Total |
| | N = 43 | N = 42 | N = 85 |
| Analysis Set | n (%) | n (%) | n (%) |
| Full analysis set | 43 (100) | 42 (100) | 85 (100) |
| Treated with macitentan | 43 (100) | 0 | 43 (50.6) |
| Treated with placebo | 0 | 42 (100) | 42 (49.4) |
| Per-protocol analysis set | 36 (83.7) | 37 (88.1) | 73 (85.9) |
| Treated with macitentan | 36 (83.7) | 0 | 36 (42.4) |
| Treated with placebo | 0 | 37 (88.1) | 37 (43.5) |
| Safety set | 43 (100) | 42 (100) | 85 (100) |
| Treated with macitentan | 43 (100) | 0 | 43 (50.6) |
| Treated with placebo | 0 | 42 (100) | 42 (49.4) |
| *Pharmacokinetic analysis set | 7 (16.3) | 3 (7.1) | 10 (11.8) |
| Treated with macitentan | 7 (16.3) | 0 | 7 (8.2) |
| Treated with placebo | 0 | 3 (7.1) | 3 (3.5) |
| *Macitentan Treated set | 43 (100) | 41 (97.6) | 84 (98.8) |
| Treated with macitentan | 43 (100) | 41 (97.6) | 84 (98.8) |

The percentages are based on N.
*For the pharmacokinetic analysis set and the macitentan treated set, the macitentan and placebo columns show the proportions of patients who received macitentan and placebo during the DB period

C. Protocol Deviations

All 85 patients had at least one protocol deviation during the study. During the study, all but 2 patients had at least one protocol deviation that was defined as ‘important.’ See, Table 4. Very few of these deviations were associated with the assessment of efficacy, with the most frequently reported being isolated, missing safety laboratory variables at screening (that were not required to assess eligibility) and/or at randomization (macitentan 46.5%, placebo 54.8%) and safety laboratory assessments not performed as per protocol after randomization (macitentan 90.7%, placebo 88.1%). These were generally associated with missing laboratory values or laboratory assessments performed outside of the protocol-defined window (i.e., more than 4 days) and therefore did not impact on the evaluation of efficacy. Furthermore, as only isolated safety variables were missing, this did not impact on the safety or wellbeing of the patients. Therefore, the integrity of the study was not affected. Other important protocol deviations included informed consent process not followed as per protocol (macitentan: 4.7%, placebo: 9.5%), patients randomized prior to availability of central laboratory values (macitentan: 7.0%, placebo: 7.1%), patients not compliant with study medication (macitentan: 9.3%, placebo: 14.3%), study treatment not interrupted or discontinued according to the protocol-specified criteria (macitentan: 4.7%, placebo: 2.4%), PVR at Screening missing or <4 WU or <320 dyn·s·cm⁻⁵ (macitentan: 4.7%, placebo: 2.4%). See, Table 4. Unblinding occurred for 3 patients who had SUSARs. No other code breaks or unblinding of treatment prior to database lock occurred during the study.

TABLE 4
Important protocol deviations, Randomized set

| | Macitentan | | |
|---|---|---|---|
| | 10 mg | Placebo | Total |
| | N = 43 | N = 42 | N = 85 |
| | n (%) | n (%) | n (%) |
| Subjects with at least one important | 42 (97.7) | 41 (97.6) | 83 (97.6) |
| protocol deviation | | | |
| Deviation at informed consent | 2 (4.7) | 4 (9.5) | 6 (7.1) |
| Informed consent process not followed | 2 (4.7) | 4 (9.5) | 6 (7.1) |
| At Randomization | 25 (58.1) | 24 (57.1) | 49 (57.6) |
| Other safety laboratory parameter | 20 (46.5) | 23 (54.8) | 43 (50.6) |
| missing at Screening and/or | | | |
| randomization | | | |
| Patient randomized while central lab | 3 (7.0) | 3 (7.1) | 6 |
| (7.1) values for eligibility unavailable | | | |
| PVR at Screening missing or <4 | 2 (4.7) | 1 (2.4) | 3 (3.5) |
| W.U. or <320 dyn.s.cm⁻⁵ (Inclusion 3) | | | |
| Treatment with calcium channel | 2 (4.7) | 1 (2.4) | 3 (3.5) |
| blocker, ERA, i.v./s.c., or oral | | | |
| prostanoids within 3 months | | | |
| prior to randomization | | | |
| (Exclusion 20) | | | |
| Date of Screening RHC missing | 1 (2.3) | 1 (2.4) | 2 |
| (2.4) or >28 days prior to randomization | | | |
| Initiation, change in dose or | 1 (2.3) | 0 | 1 |
| (1.2) discontinuation of PDE5i, sGC | | | |
| stimulator within 3 months prior | | | |
| to randomization (Exclusion 21) | | | |
| Stratification in IxRS not per protocol | 1 (2.3) | 1 (2.4) | 2 (2.4) |
| After Randomization | 40 (93.0) | 38 (90.5) | 78 (91.8) |
| Safety labs not performed per protocol | 39 (90.7) | 37 (88.1) | 76 (89.4) |
| after Randomization | | | |
| Post-baseline deviation | 6 (14.0) | 3 (7.1) | 9 |
| (10.6) affecting primary | | | |
| efficacy endpoints: | | | |
| Last dose of double-blind study | | | |
| drug taken on day of Week 12 | | | |
| visit before RHC | | | |
| Subject not compliant with study | 4 (9.3) | 6 (14.3) | 10 |
| (11.8) medication | | | |
| Receipt of prohibited concomitant | 3 (7.0) | 2 (4.8) | 5 |
| (5.9) medication or prohibited dose | | | |
| change/discontinuation of existing | | | |
| medication affecting the | | | |
| primary endpoint (during | | | |
| double-blind treatment period) | | | |
| Post-baseline method of assessing liver | 2 (4.7) | 6 (14.3) | 8 |
| (9.4) disease severity different to | | | |
| Screening or not performed | | | |
| Study drug not interrupted or | 2 (4.7) | 1 (2.4) | 3 |
| (3.5) permanently stopped according | | | |
| to protocol criteria | | | |
| Post-baseline deviation affecting | 1 (2.3) | 0 | 1 |
| (1.2) primary endpoint: mPAP, CO or | | | |
| PAWP/LVEDP missing at Week 12 | | | |
| Post-baseline deviation not affecting | 1 (2.3) | 1 (2.4) | 2 |
| (2.4) primary efficacy endpoint: Last | | | |
| dose of double-blind study drug taken | | | |
| on day of Week 12 assessment after RHC | | | |
| Pregnancy test not performed per | 1 (2.3) | 1 (2.4) | 2 |
| (2.4) protocol after Randomization | | | |
| Receipt of prohibited concomitant | 1 (2.3) | 0 | 1 |
| (1.2) medication not affecting the | | | |
| primary endpoint (any treatment period) | | | |
| PD in PK sub-study | 2 (4.7) | 0 | 2 (2.4) |
| Study treatment not taken per | 2 (4.7) | 0 | 2 (2.4) |
| protocol on Day 2 | | | |

D. Demographic and Baseline Characteristics

The demographic characteristics in the FAS were comparable between the macitentan and placebo groups. See, Table 5. Overall, the proportion of males (51.8%) and females (48.2%) was similar. Median age at screening was 57 years (range: 40-82 years) in the macitentan group and 61 years (range: 25-73 years) in the placebo group, with 81.4% and 69.0% of patients aged <65 years in the macitentan and placebo groups, respectively. Of the female patients enrolled, only 4 were of childbearing potential. Overall, mean BMI was 29.17 kg/m². Most of the patients were enrolled at centers in Europe (67.1%) or North America (27.1%), with 5 patients (5.9%) enrolled in Latin America. Overall, as per stratification at the time of randomization, 63.5% of patients were receiving a PAH-specific therapy at baseline. The demographic characteristics in the PPS were consistent with those in the FAS.

TABLE 5
Demographic characteristics, Full analysis set

| | Macitentan | | |
|---|---|---|---|
| | 10 mg | Placebo | Total |
| | N = 43 | N = 42 | N = 85 |
| Sex [n (%)] | | | |
| Male | 22 (51.2) | 22 (52.4) | 44 (51.8) |
| Female | 21 (48.8) | 20 (47.6) | 41 (48.2) |
| Age (years) | | | |
| n | 43 | 42 | 85 |
| Mean | 58.0 | 59.0 | 58.5 |
| SD | 8.7 | 9.5 | 9.1 |
| Median | 57 | 61 | 58 |
| Q1, Q3 | 52, 64 | 52, 66 | 52, 64 |
| Min, Max | 40, 82 | 25, 73 | 25, 82 |
| Age [n (%)] | | | |
| <65 | 35 (81.4) | 29 (69.0) | 64 (75.3) |
| >= 65 | 8 (18.6) | 13 (31.0) | 21 (24.7) |
| BMI (kg/m²) | | | |
| n | 43 | 42 | 85 |
| Mean | 29.01 | 29.33 | 29.17 |
| SD | 4.79 | 4.04 | 4.41 |
| Median | 30.2 | 28.9 | 29.0 |
| Q1, Q3 | 25.3, 32.2 | 26.4, 31.3 | 25.7, 32.0 |
| Min, Max | 18.5, 42.6 | 22.6, 39.5 | 18.5, 42.6 |
| Race [n (%)] | | | |
| Asian | 1 (2.3) | 0 | 1 (1.2) |
| White | 23 (53.5) | 21 (50.0) | 44 (51.8) |
| Other | 1 (2.3) | 0 | 1 (1.2) |
| Not Applicable | 18 (41.9) | 21 (50.0) | 39 (45.9) |
| Ethnicity [n (%)] | | | |
| Hispanic or Latino | 6 (14.0) | 6 (14.3) | 12 (14.1) |
| Not Hispanic or Latino | 19 (44.2) | 15 (35.7) | 34 (40.0) |
| Missing | 18 (41.9) | 21 (50.0) | 39 (45.9) |
| Female of childbearing | | | |
| potential [n (%)] | | | |
| Yes | 2 (4.7) | 2 (4.8) | 4 (4.7) |
| No | 19 (44.2) | 18 (42.9) | 37 (43.5) |
| Geographical region | | | |
| (as per stratification) [n (%)] | | | |
| Europe | 29 (67.4) | 28 (66.7) | 57 (67.1) |
| North America | 12 (27.9) | 11 (26.2) | 23 (27.1) |
| Latin America | 2 (4.7) | 3 (7.1) | 5 (5.9) |
| PAH Therapy | | | |
| (as per stratification) [n (%)] | | | |
| Yes | 27 (62.8) | 27 (64.3) | 54 (63.5) |
| No | 16 (37.2) | 15 (35.7) | 31 (36.5) |

Baseline disease characteristics in the FAS are summarized in Table 6. The median time since portal hypertension diagnosis was 23 months (range: 0-199 months) in the macitentan group and 31 months (range: 0-348 months) in the placebo group. The median time since PAH diagnosis was 7 months (range: 0-116 months) in the macitentan group and 12 months (range: 0-101 months) in the placebo group. At baseline, an RHC at rest was performed for all patients, and hemodynamic data were similar in the two treatment groups. Overall, the mean (±SD) 6MWD at baseline was 384.5±103.9 m. At baseline, patients were in WHO FC II (58.8%) or III (38.8%), with 2 patients (2.4%) in WHO FC I.

The most frequently reported causes of portal hypertension were alcoholic cirrhosis (62.4%) and hepatitis C (31.8%). The proportion of patients who had HVC at baseline was 44.2% in the macitentan group and 61.9% in the placebo group. The overall mean HVPG at baseline (based on central review in patients without a TIPS) was 10.4 mmHg. Esophageal varices and ascites were reported for 63.5% and 25.9% of patients, respectively. Approximately 9% of patients had a history of TIPS. MELD was assessed in 68 out of the 85 patients, and the mean (±SD) score was 10.4 (2.8).

The baseline disease characteristics in the PPS were consistent with those in the FAS.

TABLE 6
Baseline disease characteristics, Full analysis set

| | Macitentan | | |
|---|---|---|---|
| | 10 mg | Placebo | Total |
| | N = 43 | N = 42 | N = 85 |
| Time since portal hypertension | | | |
| diagnosis (months) | | | |
| n | 43 | 42 | 85 |
| Mean | 44.5 | 47.3 | 45.9 |
| SD | 48.5 | 63.6 | 56.1 |
| Median | 23 | 31 | 25 |
| Q1, Q3 | 5, 80 | 4, 69 | 5, 76 |
| Min, Max | 0, 199 | 0, 348 | 0, 348 |
| Time since PAH | | | |
| diagnosis (months) | | | |
| n | 43 | 42 | 85 |
| Mean | 23.3 | 22.9 | 23.1 |
| SD | 32.0 | 27.2 | 29.5 |
| Median | 7 | 12 | 10 |
| Q1, Q3 | 2, 33 | 1, 37 | 2, 36 |
| Min, Max | 0, 116 | 0, 101 | 0, 116 |
| Right Heart | | | |
| catheterization | | | |
| performed [n (%)] | | | |
| Yes | 43 (100) | 42 (100) | 85 (100) |
| PVR (calculated) | | | |
| at RHC (dyn.sec/cm⁵) | | | |
| n | 43 | 42 | 85 |
| Mean | 552.4 | 521.7 | 537.2 |
| SD | 192.8 | 163.3 | 178.4 |
| Median | 491 | 492 | 491 |
| Q1, Q3 | 429, 677 | 417, 593 | 429, 615 |
| Min, Max | 178, 1046 | 253, 1069 | 178, 1069 |
| Six-minute walk | | | |
| distance at baseline (m) | | | |
| n | 43 | 42 | 85 |
| Mean | 385.8 | 383.2 | 384.5 |
| SD | 100.0 | 108.9 | 103.9 |
| Median | 399 | 376 | 378 |
| Q1, Q3 | 327, 450 | 295, 448 | 320, 450 |
| Min, Max | 222, 614 | 120, 591 | 120, 614 |
| WHO functional | | | |
| class at baseline [n (%)] | | | |
| I | 1 (2.3) | 1 (2.4) | 2 (2.4) |
| II | 27 (62.8) | 23 (54.8) | 50 (58.8) |
| III | 15 (34.9) | 18 (42.9) | 33 (38.8) |
| NT pro-BNP at baseline (ng/L) | | | |
| n | 41 | 40 | 81 |
| Missing | 2 | 2 | 4 |
| Mean | 488.0 | 367.5 | 428.4 |
| SD | 833.1 | 598.1 | 724.6 |
| Median | 187 | 151 | 160 |
| Q1, Q3 | 83, 471 | 70, 357 | 72, 442 |
| Min, Max | 51, 4476 | 51, 3374 | 51, 4476 |
| Child-Pugh classification | | | |
| at baseline [n (%)] | | | |
| A | 20 (46.5) | 17 (40.5) | 37 (43.5) |
| B | 3 (7.0) | 8 (19.0) | 11 (12.9) |
| Not classified | 20 (46.5) | 17 (40.5) | 37 (43.5) |
| MELD score at baseline | | | |
| n | 35 | 33 | 68 |
| Missing | 8 | 9 | 17 |
| Mean | 10.3 | 10.4 | 10.4 |
| SD | 2.8 | 2.9 | 2.8 |
| Median | 10 | 10 | 10 |
| Q1, Q3 | 8, 12 | 8, 12 | 8, 12 |
| Min, Max | 6, 17 | 6, 18 | 6, 18 |
| Liver biopsy | | | |
| performed [n (%)] (*) | | | |
| Yes | 7 (16.3) | 9 (21.4) | 16 (18.8) |
| No | 35 (81.4) | 32 (76.2) | 67 (78.8) |
| Missing | 1 (2.3) | 1 (2.4) | 2 (2.4) |
| Hepatic vein | | | |
| catheterization performed | | | |
| [n (%)] (*) | | | |
| Yes | 19 (44.2) | 26 (61.9) | 45 (52.9) |
| No | 23 (53.5) | 16 (38.1) | 39 (45.9) |
| Missing | 1 (2.3) | 0 | 1 (1.2) |
| Eosophageal varices [n (%)] (*) | | | |
| Absent | 17 (39.5) | 14 (33.3) | 31 (36.5) |
| Present | 26 (60.5) | 28 (66.7) | 54 (63.5) |
| Ascites [n (%)] (*) | | | |
| Absent | 30 (69.8) | 32 (76.2) | 62 (72.9) |
| Present | 12 (27.9) | 10 (23.8) | 22 (25.9) |
| Missing | 1 (2.3) | 0 | 1 (1.2) |
| History of TIPS [n (%)] (*) | | | |
| Absent | 40 (93.0) | 37 (88.1) | 77 (90.6) |
| Present | 3 (7.0) | 5 (11.9) | 8 (9.4) |
| Cause of Portal | | | |
| Hypertension [n (%)] (*) | | | |
| Total | 43 (100) | 42 (100) | 85 (100) |
| Autoimmune hepatitis | 3 (7.0) | 1 (2.4) | 4 (4.7) |
| Biliary cirrhosis primary | 1 (2.3) | 1 (2.4) | 2 (2.4) |
| Cirrhosis alcoholic | 27 (62.8) | 26 (61.9) | 53 (62.4) |
| Cryptogenic cirrhosis | 1 (2.3) | 0 | 1 (1.2) |
| Hepatitis B | 1 (2.3) | 1 (2.4) | 2 (2.4) |
| Hepatitis C | 11 (25.6) | 16 (38.1) | 27 (31.8) |
| Metabolic syndrome | 1 (2.3) | 0 | 1 (1.2) |
| Non-alcoholic steatohepatitis | 2 (4.7) | 5 (11.9) | 7 (8.2) |
| Portal vein thrombosis | 1 (2.3) | 0 | 1 (1.2) |
| Hepatic Venous Pressure | | | |
| Gradient at baseline (mmHg) | | | |
| n | 28 | 27 | 55 |
| Missing | 15 | 15 | 30 |
| Mean | 9.8 | 9.5 | 9.6 |
| SD | 3.6 | 4.2 | 3.9 |
| Median | 9 | 10 | 9 |
| Q1, Q3 | 7, 13 | 7, 13 | 7, 13 |
| Min, Max | 3, 17 | 1, 17 | 1, 17 |
| Hepatic Venous Pressure | | | |
| Gradient (Central review) | | | |
| at baseline (mmHg) | | | |
| n | 16 | 14 | 30 |
| Missing | 27 | 28 | 55 |
| Mean | 10.6 | 10.3 | 10.4 |
| SD | 3.4 | 3.9 | 3.6 |
| Median | 10 | 10 | 10 |
| Q1, Q3 | 8, 12 | 7, 14 | 8, 13 |
| Min, Max | 6, 20 | 5, 17 | 5, 20 |

(*) At time of portal hypertension diagnosis.

E. Previous and Concomitant Diseases at Baseline

The most frequently reported ongoing conditions were associated with the SOCs of Hepatobiliary disorders (95.3%), Gastrointestinal disorders (87.1%), Metabolism and nutrition disorders (75.3%), and Respiratory, thoracic and mediastinal disorders (54.1%).

F. Previous and Concomitant Therapy at Baseline

The proportion of patients who had received at least one previous therapy (i.e., a treatment for which the end date was prior to the start of the DB treatment period) in the 3 months prior to randomization was 11.6% in the macitentan group and 33.3% in the placebo group. All patients were receiving at least one study-concomitant therapy at baseline (i.e., treatments ongoing at study start), except 1 patient. These included diuretics (e.g., furosemide, spironolactone), proton pump inhibitors (e.g., omeprazole, esomeprazole), beta blockers, potassium supplements, and liver therapies. A total of 64.7% of patients were receiving at least one PAH-specific therapy at study start, with an oral PDE-5 inhibitor reported for all these patients; either oral PDE-5 inhibitors (64.7%) and/or prostanoids (5.9%). See, Table 7.

TABLE 7
Study concomitant PAH-specific therapies at baseline, Full analysis set

| | Macitentan | | |
|---|---|---|---|
| | 10 mg | Placebo | Total |
| Specific therapy | N = 43 | N = 42 | N = 85 |
| Preferred Term | n (%) | n (%) | n (%) |
| Subjects with at least one | 28 (65.1) | 27 (64.3) | 55 (64.7) |
| specific PAH therapy | | | |
| Prostanoid | 4 (9.3) | 1 (2.4) | 5 (5.9) |
| TREPROSTINIL SODIUM | 2 (4.7) | 1 (2.4) | 3 (3.5) |
| ILOPROST | 2 (4.7) | 0 | 2 (2.4) |
| Oral PDE5i | 28 (65.1) | 27 (64.3) | 55 (64.7) |
| SILDENAFIL | 10 (23.3) | 11 (26.2) | 21 (24.7) |
| SILDENAFIL CITRATE | 9 (20.9) | 9 (21.4) | 18 (21.2) |
| TADALAFIL | 9 (20.9) | 7 (16.7) | 16 (18.8) |

Per-protocol, PAH advanced therapy covers oral or inhaled prostanoids, endothelin receptor antagonists, PDE-5 inhibitors, and soluble guanylate cyclase stimulators. In addition, one patient was enrolled who was receiving treatment with subcutaneous treprostinil sodium (protocol deviation).

Efficacy Evaluation

A. Measurements of Treatment Compliance

Treatment compliance was evaluated by study drug accountability.

During the DB treatment period, study treatment compliance between 80% to 120% was recorded for 97.7% and 97.6% of patients in the macitentan and placebo groups, respectively. In the MTS, study treatment compliance between 80% to 120% was recorded for 97.6% of patients.

B. Efficacy Results

(i) Primary Endpoint(s)

Main Analysis

The primary endpoint was relative change from baseline to Week 12 in PVR, expressed as ratio of Week 12 to baseline PVR. In the FAS (N=85 including 5 patients with imputed values), the geometric mean (95% CLs) ratios of Week 12 to baseline PVR were 0.63 (0.58, 0.67) and 0.98 (0.91, 1.05) in the macitentan and placebo groups, respectively. See, Table 8.

TABLE 8
Change from baseline to Week 12 in
PVR, Full analysis set, with imputation

| | Macitentan | |
|---|---|---|
| | 10 mg | Placebo |
| | N = 43 | N = 42 |
| PVR (dyn.sec/cm⁵) | | |
| Baseline | | |
| n | 43 | 42 |
| Mean | 552.4 | 521.7 |
| SD | 192.82 | 163.28 |
| Median | 490.5 | 492.4 |
| Q1, Q3 | 429.4, 676.5 | 416.7, 592.6 |
| Min, Max | 178, 1046 | 253, 1069 |
| Week 12 | | |
| n | 43 | 42 |
| Mean | 350.3 | 514.5 |
| SD | 133.41 | 170.40 |
| Median | 328.6 | 479.6 |
| Q1, Q3 | 260.7, 413.8 | 397.9, 626.6 |
| Min, Max | 108, 625 | 218, 950 |
| Total number of patients | 4 (9.3) | 1 (2.4) |
| with imputation of | | |
| missing values [n (%)] | | |
| due to death (by max ratio observed) (a) | 0 | 0 |
| by median ratio observed (a) | 3 (7.0) | 1 (2.4) |
| by last observation carried forward | 1 (2.3) | 0 |
| Change from baseline to Week 12 | | |
| n | 43 | 42 |
| Mean | −202.1 | −7.2 |
| SD | 123.56 | 122.20 |
| Median | −179.9 | −22.6 |
| Q1, Q3 | −306.5, −114.8 | −74.9, 57.8 |
| Min, Max | −459, 56 | −276, 364 |
| Ratio of baseline | | |
| n | 43 | 42 |
| Geometric mean | 0.63 | 0.98 |
| Geometric CV | 0.24 | 0.23 |
| 95% CL of geometric mean | 0.58, 0.67 | 0.91, 1.05 |
| Median | 0.60 | 0.96 |
| TREATMENT EFFECT | | |
| Geometric mean ratio | 0.64 | |
| 95% CL of geometric mean ratio | 0.58, 0.71 | |

(a) Ratio observed = Ratio of baseline PVR observed in the same treatment group times baseline value of the patient

The PVR ratios of Week 12 to baseline PVR were log-transformed and an ANCOVA was performed using treatment, background PAH-specific therapy at baseline and region as factors, and log-transformed PVR at baseline as a covariate. From the adjusted model, the treatment effect at Week 12 (geometric mean ratio macitentan over placebo) was 0.65 (p<0.0001), i.e., a 35% reduction in PVR in the macitentan group compared to the placebo group. See, Table 9.

TABLE 9
Between-treatment analysis of PVR at Week 12, Full analysis set

| | | | | | | | Macitentan |
|---|---|---|---|---|---|---|---|
| | | | | | Macitentan | | 10 mg- |
| | NDF | DDF | F-value | P-value | 10 mg | Placebo | Placebo |
| Number of patients included | | | | | 43 | 42 | |
| in the analysis set | | | | | | | |
| Number of patients included | | | | | 43 | 42 | |
| in the analysis | | | | | | | |
| Type III analysis of effects | | | | | | | |
| Treatment | 1 | 79 | 76.31 | <.0001 | | | |
| LS mean | | | | | −0.41 | 0.03 | −0.43 |
| SE | | | | | 0.05 | 0.05 | 0.05 |
| 95% CL | | | | | −0.50, −0.31 | −0.07, 0.12 | −0.53, −0.33 |
| Model-adjusted | | | | | 0.67 | 1.03 | |
| geometric mean | | | | | | | |
| 95% CL of model- | | | | | 0.61, 0.73 | 0.93, 1.13 | |
| adjusted geometric | | | | | | | |
| mean | | | | | | | |
| Model-adjusted geometric mean ratio | | | | | | | 0.65 |
| 95% CL of model-adjusted geometric mean ratio | | | | | | | 0.59, 0.72 |

Statistical model is Analysis of Covariance including log(PVR at baseline) as a covariate, with Treatment, background PAH-specific therapy at baseline and region as factors in the model. Dependent variable is log (ratio of baseline PVR at Week 12).

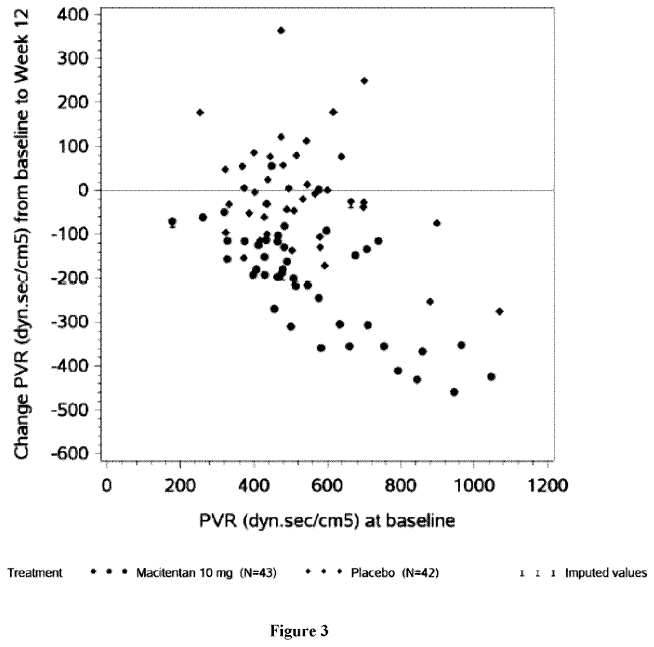

The null hypothesis was rejected as the 2-sided 95% confidence interval did not include 1.0. The mean (±SD) decrease in PVR was 202.1 (±123.56) dyn·sec/cm⁵ in the macitentan group and 7.2 (±122.20) dyn·sec/cm⁵ in the placebo group. See, Table 8. In the macitentan group, only 1 patient had an increase in PVR and 1 patient had no change. All other patients had decreases. In the placebo group, approximately 40% of patients had an increase in PVR. See, FIG. 3. The observed effect on PVR across demographic and baseline disease characteristic subgroups in the FAS was consistent with the overall treatment effect, with no indication of heterogeneity.

Supportive/Sensitivity Analyses

Results of the sensitivity analyses on the primary endpoint were consistent with that of the main analysis. See, Tables 8 and 10.

TABLE 10
Results of the supportive/sensitivity analyses on the primary endpoint of PVR

| | | | Geometric mean |
|---|---|---|---|
| | | Difference in LS | ratio of macitentan |
| | | means (95% CLs) | versus placebo |
| Analyses | Statistical model | macitentan-placebo | (95% CLs) |
| Sensitivity analysis | ANCOVA model | | 0.65 (0.59, 0.72), |
| on observed cases, | adjusted for | | p <0.0001 |
| FAS (N = 80) | treatment, | | |
| | background PAH- | | |
| | specific therapy at | | |
| | baseline, and region | | |
| | as factors and log- | | |
| | transformed PVR at | | |
| | baseline as a | | |
| | covariate. | | |
| | Dependent variable | | |
| | is log (ratio of | | |
| | baseline PVR at | | |
| | Week 12). | | |
| Sensitivity analysis | ANCOVA model | −180.71 dyn.sec/cm⁵ | |
| on change from | adjusted for | (−224.47, −136.96), | |
| baseline (no log- | treatment, | p <0.0001 | |
| transformation), | background PAH- | | |
| FAS (N = 85) | specific therapy at | | |
| | baseline, and region | | |
| | as factors and PVR | | |
| | at baseline as a | | |
| | covariate. | | |
| Sensitivity analysis | ANCOVA model | | 0.65 (0.58, 0.71), |
| without adjustment | adjusted for | | p <0.0001 |
| for stratification | treatment as a factor | | |
| factors, | and log-transformed | | |
| FAS (N = 85) | PVR at baseline as a | | |
| | covariate. | | |
| | Dependent variable | | |
| | is log (ratio of | | |
| | baseline PVR at | | |
| | Week 12). | | |
| Sensitivity analysis | ANCOVA model | | 0.65 (0.59, 0.72), |
| with slope | adjusted for | | p <0.0001 |
| imputation, | treatment, | | |
| FAS (N = 85) | background PAH- | | |
| | specific therapy at | | |
| | baseline, and region | | |
| | as factors and log- | | |
| | transformed PVR at | | |
| | baseline as a | | |
| | covariate. | | |
| | Dependent variable | | |
| | is log (ratio of | | |
| | baseline PVR at | | |
| | Week 12). | | |
| Main analysis using | ANCOVA model | | 0.64 (0.58, 0.71), |
| the PPS (N = 73) | adjusted for | | p <0.0001 |
| | treatment, | | |
| | background PAH- | | |
| | specific therapy at | | |
| | baseline, and region | | |
| | as factors and log- | | |
| | transformed PVR at | | |
| | baseline as a | | |
| | covariate. | | |
| | Dependent variable | | |
| | is log (ratio of | | |
| | baseline PVR at | | |
| | Week 12). | | |
| Sensitivity analysis | ANCOVA model | | 0.69 (0.61, 0.77), |
| with alternative | adjusted for | | p <0.0001 |
| imputation, | treatment, | | |
| FAS (N = 85)- | background PAH- | | |
| Performed as post- | specific therapy at | | |
| hoc analysis | baseline, and region | | |
| | as factors and log- | | |
| | transformed PVR at | | |
| | baseline as a | | |
| | covariate. | | |
| | Dependent variable | | |
| | is log (ratio of | | |
| | baseline PVR at | | |
| | Week 12). The 4 | | |
| | Macitentan patients | | |
| | that discontinued | | |
| | treatment due to AE | | |
| | are imputed by | | |
| | group maximum | | |
| | ratio of baseline in | | |
| | their treatment | | |
| | group. | | |

Subgroup Analyses

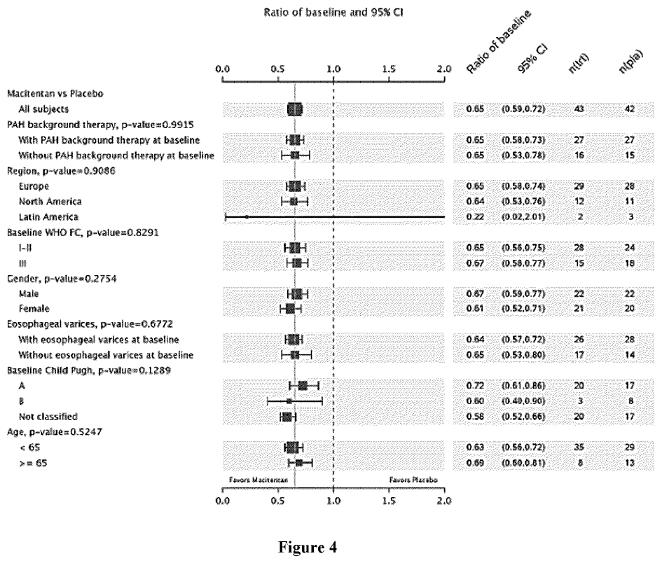

The primary endpoint of PVR was also evaluated across different subgroups. The PAH background therapy at baseline and the region subgroups were defined in the protocol. A forest plot of relative change from baseline to Week 12 in PVR by subgroups is presented in FIG. 4. The observed treatment effect (model-adjusted geometric mean ratio of macitentan versus placebo) across subgroups was consistent with the overall treatment effect, with no indication of heterogeneity.

(ii) Secondary Endpoints and Related Other Efficacy Endpoints

6MWD

The secondary efficacy endpoints were:
Change from Baseline to Week 12 in mean right atrial pressure (mRAP), mean pulmonary arterial pressure (mPAP), cardiac index, total pulmonary resistance (TPR), and mixed venous oxygen saturation (SVO₂), all measured at rest
Change from Baseline to Week 12 in WHO FC
Change from Baseline to Week 12 in 6MWD
Change from Baseline to Week 12 in N-terminal pro b-type natriuretic peptide (NT-proBNP).

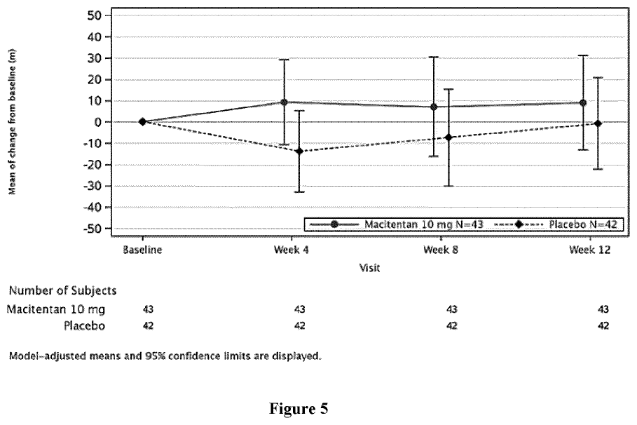

No adjustment was made for multiplicity for secondary endpoints, therefore all corresponding p-values provided are of an exploratory nature. Mean changes from baseline to Weeks 4, 8, and 12 in 6MWD (FAS with imputation) are summarized in Table 11 and graphically presented in FIG. 5. The main analysis on 6MWD was performed using an MMRM adjusted for treatment, visit, region, PAH-specific therapy at baseline, and treatment-by-visit interaction as factors, and baseline 6MWD and WHO FC as covariates. See, Table 11.

TABLE 11
Change from baseline to Weeks 4, 8, and 12 in 6MWD, Full analysis set

| | | | Repeated-measures |
|---|---|---|---|
| | | | (MMRM) between |
| | | | treatment analysis |
| | | | of 6MWD^{a} |
| | | | Difference |
| | Macitentan | Placebo | in LS means |
| | N = 43 | N = 42 | (95% CLs) |
| Time point | mean ± SD | mean ± SD | macitentan-placebo |
| Baseline | 385.8 ± 99.97 m | 383.2 ± 108.90 m | |
| n (imputed) | 43 (2) | 42 (1) | |
| Week 4 | 392.5 ± 93.74 m | 367.8 ± 118.45 m | |
| Change from | 6.7 ± 44.55 m | −15.4 ± 47.86 m | 23.02 m |
| baseline to | | | (3.38, 42.67), |
| Week 4 | | | p = 0.0222 |
| n (imputed) | 43 (4) | 42 (2) | |
| Week 8 | 390.3 ± 99.35 m | 374.2 ± 120.70 m | |
| Change from | 4.5 ± 67.40 m | −9.0 ± 53.36 m | 14.47 m |
| baseline to | | | (−11.90, 40.85), |
| Week 8 | | | p = 0.2780 |
| n (imputed) | 43 (5) | 42 (2) | |
| Week 12 | 392.2 ± 98.46 m | 380.8 ± 114.98 m | |
| Change from | 6.4 ± 65.74 m | −2.4 ± 43.65 m | 9.73 m |
| baseline to | | | (−14.50, 33.95), |
| Week 12 | | | p = 0.4264 |
| | | | Overall: 15.74 m |
| | | | (−5.62, 37.10) |

^{a}Statistical model is Repeated-measures Analysis of Covariance adjusted for treatment, visit, region, PAH-specific therapy at baseline, and treatment-by-visit interaction as factors and 6MWD at baseline and WHO FC as covariates. Random variables are the patient-intercept and the patient-by-visit interaction. Dependent variable is change in 6MWD at the respective week.

The least square (LS) mean difference (macitentan−placebo) of change from baseline up to Week 12 in 6MWD was 9.73 m (95% CLs: −14.50, 33.95), p=0.4264. The values were imputed for 7 (5 macitentan, 2 placebo) patients at Week 12. Results of the MMRM without adjustment for stratification factors were similar to those with stratification factors described above. 6MWD at Week 12 was also analyzed as a sensitivity analysis using an ANCOVA model adjusted for treatment, region and PAH-specific therapy at baseline as factors, and 6MWD at baseline as a covariate. In this analysis, the LS mean difference (macitentan−placebo) of change from baseline to Week 12 in 6MWD was consistent with the main analysis at 10.05 m (95% CLs: −13.74, 33.84), p=0.4029. The mean (±SD) observed difference at Week 12 (macitentan−placebo) was 18.4 m (43.54), which was higher than in the main analysis, due to fact that no imputation was considered (imputation decreased the treatment effect in the main See, Table 12.

TABLE 12
Results of sensitivity analyses on the primary endpoint of PVR

| | Geometric mean ratio |
|---|---|
| | (macitentan vs placebo, |
| Analyses | 95% CLs), p-value |
| Primary analysis (N = 85) | 0.65 (0.59, 0.72), p < 0.0001 |
| ANCOVA without stratification | 0.65 (0.58, 0.71), p < 0.0001 |
| factors (N = 85) | |
| ANCOVA (observed cases) (N = 80) | 0.65 (0.59, 0.72), p < 0.0001 |
| ANCOVA PPS (N = 73) | 0.65 (0.58, 0.71), p < 0.0001 |
| ANCOVA (worse case imputation*) (N = 85) | 0.65 (0.61, 0.77), p < 0.0001 |
| | LS Mean difference, meters |
| | (macitentan-placebo, 95%, |
| | CLs, p-value) |
| ANCOVA (Absolute Change) | −180.71 (−224.47, |
| (N = 85) | −136.97), p < 0.0001 |

*The 4 macitentan patients that discontinued treatment due to AE or lack of efficacy are imputed using the maximum ratio of baseline in their treatment group.

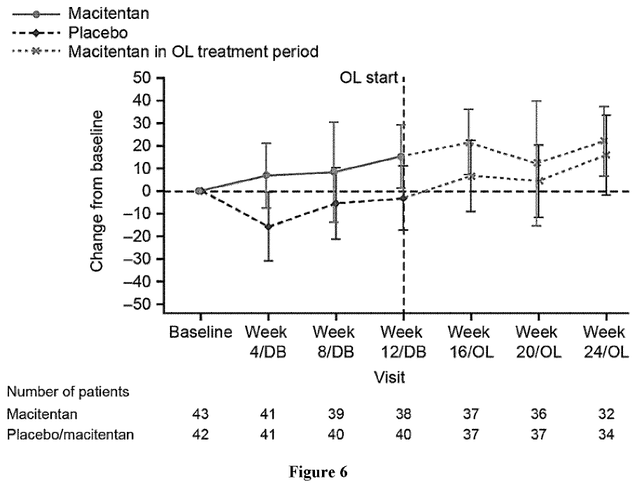

For patients who had received macitentan in the DB and/or OL treatment periods, changes from macitentan baseline to each (available) time point in 6MWD were analyzed using the MTS without imputation. See, FIG. 6. In this analysis, for patients who had received macitentan in both treatment periods, mean (±SD) changes from baseline ranged from 7.0±45.62 at Week 4 to 22.0±42.55 m at Week 24. For patients who had received macitentan only in the OL treatment period (DB placebo-treated patients), mean (±SD) changes from macitentan baseline in the OL treatment period ranged from 11.5±31.67 at Week 4 to 13.9±39.27 m at Week 12.

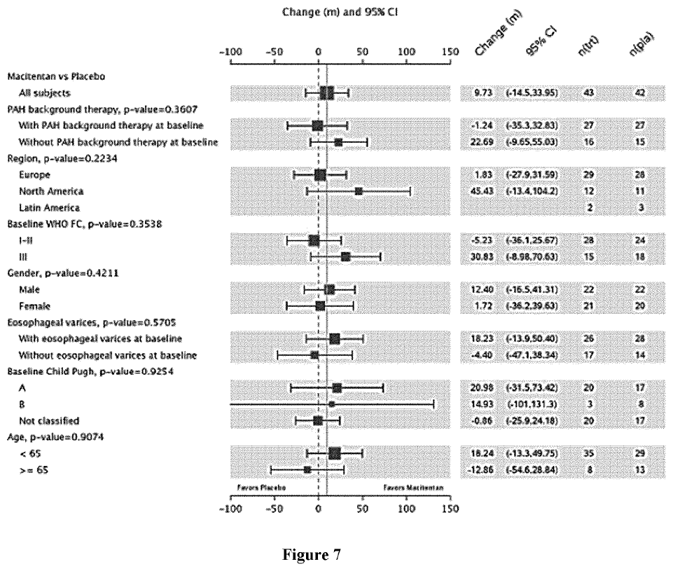

6MWD was evaluated across the subgroups. A forest plot of changes from baseline to Week 12 in 6MWD subgroups is presented in FIG. 7.

WHO FC

Change from Baseline to Week 12 in WHO FC was a secondary endpoint. At Week 12, 65.1% and 81.0% of patients in the macitentan and placebo groups, respectively, had no change in their WHO FC status compared to baseline. See, Table 13.

Improvement in WHO FC at Week 12 was reported for 9 (20.9%) patients in the macitentan group and 7 (16.7%) patients in the placebo group. Over the same period, worsening in WHO FC was reported for 6 (14.0%) patients in the macitentan group (although 4 of these cases were imputed due to missing values) and 1 (2.4%) patient in the placebo group. A logistic regression model (exact) adjusted for treatment, PAH-specific therapy at baseline, and region as covariates was used to analyze worsening in WHO FC. At Week 12, odds ratio (macitentan versus placebo) for worsening in WHO FC was 6.253 (95% CLs: 0.714, 298.376), p=0.1278.

TABLE 13
Shift table of change from baseline to Week 12 in WHO FC, Full analysis set

| | | | Week 12 | | | | |
|---|---|---|---|---|---|---|---|
| | | | Class I | Class II | Class III | Class IV | Missing |
| Treatment | | Baseline | n (%) | n (%) | n (%) | n (%) | n (%) |
| Macitentan 10 mg | (N = 43) | Class I | 1 (2.3) | 0 | 0 | 0 | 0 |
| | | Class II | 1 (2.3) | 20 (46.5) | 6 (14.0) | 0 | 0 |
| | | Class III | 1 (2.3) | 7 (16.3) | 7 (16.3) | 0 | 0 |
| | | Class IV | 0 | 0 | 0 | 0 | 0 |
| | | Missing | 0 | 0 | 0 | 0 | 0 |
| | | Total | 3 (7.0) | 27 (62.8) | 13 (30.2) | 0 | 0 |
| Placebo | (N = 42) | Class I | 1 (2.4) | 0 | 0 | 0 | 0 |
| | | Class II | 3 (7.1) | 19 (45.2) | 1 (2.4) | 0 | 0 |
| | | Class III | 0 | 4 (9.5) | 14 (33.3) | 0 | 0 |
| | | Class IV | 0 | 0 | 0 | 0 | 0 |
| | | Missing | 0 | 0 | 0 | 0 | 0 |
| | | Total | 4 (9.5) | 23 (54.8) | 15 (35.7) | 0 | 0 |

| | Macitentan | |
|---|---|---|
| | 10 mg | Placebo |
| | N = 43 | N = 42 |
| n | 43 | 42 |
| Worsened | 6 (14.0) | 1 (2.4) |
| Not Worsened | 37 (86.0) | 41 (97.6) |
| Unchanged | 28 (65.1) | 34 (81.0) |
| Improved | 9 (20.9) | 7 (16.7) |
| Number of patients imputed | 4 (9.3) | 1 (2.4) |
| due to death (by Class IV) | 0 | 0 |
| by LOCF | 4 (9.3) | 1 (2.4) |

WHO functional class worsened, remained unchanged or improved if the class level increased, did not change or decreased, respectively. The percentages are based on N.

For patients who had received macitentan in the DB and/or OL treatment periods, changes from macitentan baseline to each (available) time point in WHO FC were analyzed using the MTS without imputation. For patients with non-missing values at Week 24 who had received macitentan in both treatment periods, no change in WHO FC status was observed for 21/34 patients (61.8%) and worsening was observed for 2/34 patients (5.9%), compared to baseline. For patients with non-missing values at Week 12 (after initiation of macitentan treatment) in the OL period, no change in WHO FC status was observed for 24/35 patients (68.6%), and worsening was observed for 3/35 patients (8.6%) compared to macitentan baseline.

RHC Variables

No adjustment was made for multiplicity for secondary endpoints, therefore all corresponding p-values provided are of an exploratory nature. The main analysis of the secondary 6MWD endpoint was performed using an MMRM adjusted for treatment, visit, region, PAH-specific therapy at baseline, and treatment-by-visit interaction as factors, and 6MWD at baseline and WHO FC as covariates. Change from Baseline to Week 12 in mRAP, mPAP, cardiac index, TPR, and SVO₂, at rest, were secondary endpoints. Changes from baseline to Week 12 in RHC variables using FAS with imputation are summarized in Table 14. The LS mean differences (macitentan−placebo) of change from baseline to Week 12 were clinically significant for mPAP (−5.99 mmHg, p<0.0001), cardiac index (0.52 L/min/m2, p=0.0009), and TPR (−171.48 dyn·sec/cm⁵ (p<0.0001). A scatter plot of change from baseline to Week 12 in mPAP for the macitentan group vs the placebo group clearly shows a clustering of macitentan-treated patients in the lower half of the figure (i.e., a decrease in mPAP) and a clustering of placebo-treated patients in the upper half (i.e., an increase in mPAP). No clinically significant effect was seen for mRAP or SVO₂. Despite the increase in cardiac index in the macitentan group, no increase in hepatic venous pressure gradient (HVPG) was observed vs placebo.

TABLE 14
Change from baseline to Week 12 in RHC variables, Full analysis set

| | | | | | | Between |
|---|---|---|---|---|---|---|
| | | | | | | treatment |
| | | | | | | analysis^{a} |
| | | | | | | Difference in |
| | | | | | | LS means |
| Right heart | | Macitentan | | Placebo | | (95% CLs) |
| catheterization | | N = 43 | | N = 42 | | macitentan- |
| variable | Time point | mean ± SD | | mean ± SD | | placebo |
| Mean right atrial | n (imputed)/missing | 42(3)/1 | | 42(1)/0 | | 1.67 |
| pressure (mRAP, | Baseline | 7.3 | ± 3.74 | 6.7 | ± 3.60 | (−0.10, 3.44), |
| mmHg) | Week 12 | 9.0 | ± 5.32 | 7.0 | ± 2.93 | p = 0.0637 |
| | Change from baseline | 1.6 | ± 5.55 | 0.3 | ± 3.29 | |
| | to Week 12 | | | | | |
| Mean pulmonary | n (imputed)/missing | 43(4)/0 | | 42(1)/0 | | −5.99 |
| artery pressure | Baseline | 46.4 | ± 7.89 | 43.8 | ± 8.52 | (−8.40, −3.57), |
| (mPAP, mmHg) | Week 12 | 40.0 | ± 7.61 | 44.2 | ± 8.26 | p < 0.0001 |
| | Change from baseline | −6.4 | ± 4.94 | 0.4 | ± 7.04 | |
| | to Week 12 | | | | | |
| Cardiac index | n (imputed)/missing | 43(4)/0 | | 42(1)/0 | | 0.52 |
| (L/min/m²) | Baseline | 3.1 | ± 0.83 | 2.9 | ± 0.76 | (0.22, 0.81), |
| | Week 12 | 3.7 | ± 1.04 | 3.0 | ± 0.82 | p = 0.0009 |
| | Change from baseline | 0.6 | ± 0.8 | 0.1 | ± 0.6 | |
| | to Week 12 | | | | | |
| Total pulmonary | n (imputed)/missing | 43(4)/0 | | 42(1)/0 | | −171.48 |
| resistance | Baseline | 689.3 | ± 228.59 | 671.5 | ± 199.73 | (-223.67, |
| (TPR, dyn · sec/cm⁵) | Week 12 | 489.4 | ± 157.13 | 653.1 | ± 197.88 | −119.30), |
| | Change from baseline | −199.8 | ± 163.06 | −18.3 | ± 135.28 | p < 0.0001 |
| | to Week 12 | | | | | |
| Mixed venous oxygen | n (imputed)/missing | 41(7)/2 | | 41(3)/1 | | 0.03 |
| saturation (SVO₂, %) | Baseline | 69.2 | ± 9.87 | 69.9 | ± 5.34 | (−2.85, 2.91), |
| | Week 12 | 70.3 | ± 7.07 | 70.7 | ± 8.58 | p = 0.9844 |
| | Change from baseline | 1.1 | ± 6.70 | 0.8 | ± 7.81 | |
| | to Week 12 | | | | | |

^{a}Statistical model is Analysis of Covariance adjusted for treatment, region, and PAH-specific therapy at baseline as factors and the RHC variable value at baseline as a covariate.

Nt-Probnp

Change from baseline to Week 12 in NT-proBNP was a secondary endpoint. At baseline, the mean (±SD) NT-proBNP was 488.0±833.07 ng/L in the macitentan group and 367.5±598.05 ng/L in the placebo group. The geometric mean ratio of Week 12 to baseline NT-proBNP was 0.86 and 1.04 in the macitentan and placebo groups, respectively. Using the ANCOVA model, the adjusted geometric mean ratio (macitentan versus placebo) at Week 12 was 0.874 (95% CLs: 0.639, 1.196), p=0.3951. NT-proBNP results using the FAS without imputation were similar to those with imputation described above.

(iii) Other Efficacy Endpoints

Hepatic venous pressure gradient: At baseline, the mean (±SD) HVPG was 9.4±3.20 mmHg in the macitentan group (n=19) and 9.9±4.00 mmHg in the placebo group (n=22). At Week 12, the mean change from baseline was −0.4 and −0.6 mmHg in the macitentan and placebo groups, respectively. HVC tracings were also reviewed by an SC member for patients with tracings available for screening and Week 12 HVCs and who did not have TIPS. According to the central review, the mean (±SD) HVPG at baseline was 10.5±3.49 mmHg in this sub-set of patients from the macitentan group (n=15) and 10.5±3.81 mmHg from the placebo group (n=11). At Week 12, the mean change from baseline was −0.5 and 1.5 mmHg in these patients from the macitentan and placebo groups, respectively.

Borg dyspnea index: At baseline, the mean (±SD) score was 3.3±2.09 in the macitentan group and 3.7±2.17 in the placebo group. Over time, no change in Borg dyspnea score was observed.

C. Pharmacokinetic Results

The PK substudy was performed in patients who had received macitentan OL treatment for at least 4 weeks and were therefore at steady state. See, Table 1. The PKS included 10 patients (previously randomized to DB macitentan: 7, DB placebo: 3). See, Table 3. However, 2 patients received their Day 2 macitentan dose prior to their scheduled pre-dose blood sampling and, therefore, trough concentration is missing for these 2 patients. The mean±SD trough plasma concentrations of macitentan and ACT-132577 (n=8) were 213.1±86.4 ng/mL and 737.4±190.0 ng/mL, respectively. Median (range) tₘₐₓ of macitentan and ACT-132577 was 6.5 h (3.0, 10.0) and 6.5 h (0.0, 24.0), respectively. The geometric means (95% confidence intervals) for Cₘₐₓ and AUC_{T} of macitentan were 368.6 ng/mL (306.9, 442.6) and 6655.4 h*ng/mL (5229.6, 8470.0), respectively. The geometric means (95% confidence intervals) for Cₘₐₓ and AUC_{T} of ACT-132577 were 869.8 ng/mL (728.2, 1038.9) and 18100.0 h*ng/mL (14795.4, 22142.9), respectively. Plasma exposure to macitentan and its active metabolite ACT-132577 were consistent between PoPH patients and patients with other forms of PAH.

Safety Evaluation

A. Extent of Exposure

(i) Exposure to Study Treatments

The median duration of DB treatment exposure was approximately 12 weeks and was similar for macitentan and placebo. More than 90% of patients in both treatment groups received treatment up to the Week 12 visit window. See, Table 15.

TABLE 15
Study treatment exposure, Safety set

| | Macitentan | |
|---|---|---|
| | 10 mg | Placebo |
| | N = 43 | N = 42 |
| Duration of study treatment during | | |
| DB period (weeks) | | |
| n | 43 | 42 |
| Mean | 11.54 | 12.01 |
| SD | 2.26 | 0.59 |
| Median | 12.1 | 12.0 |
| Q1, Q3 | 11.9, 12.4 | 11.7, 12.1 |
| Min, Max | 1.3, 13.4 | 10.4, 13.7 |
| Cumulative duration of study treatment | | |
| during DB period [n (%)] | | |
| Less than 2 weeks | 1 (2.3) | 0 |
| At least 2 weeks | 42 (97.7) | 42 (100) |
| At least 4 weeks | 42 (97.7) | 42 (100) |
| At least 8 weeks | 40 (93.0) | 42 (100) |
| Up to the Week 12 visit | 39 (90.7) | 40 (95.2) |
| window (a) | | |

(a) The Week 12 visit window was reached after 11 weeks of treatment in DB period.
Duration of study treatment was including potential treatment interruptions, while study treatment exposure was excluding treatment interruptions.

During the combined DB+OL treatment phases, the median duration of exposure to macitentan was 13.0 weeks, with approximately 73% of patients receiving treatment for at least 12 weeks. See, Table 16.

TABLE 16
Study treatment exposure, Macitentan treated set

| | Total N = 84 |
|---|---|
| Duration of study treatment | |
| during Macitentan period | |
| (weeks) | |
| n | 84 |
| Mean | 16.98 |
| SD | 7.07 |
| Median | 13.0 |
| Q1, Q3 | 11.9, 24.1 |
| Min, Max | 1.3, 28.0 |
| Cumulative duration of study | |
| treatment during Macitentan | |
| period [n (%)] | |
| Less than 2 weeks | 1 (1.2) |
| At least 2 weeks | 83 (98.8) |
| At least 4 weeks | 82 (97.6) |
| At least 8 weeks | 78 (92.9) |
| At least 12 weeks | 61 (72.6) |
| At least 16 weeks | 38 (45.2) |
| At least 20 weeks | 38 (45.2) |

For patients who entered the OL extension the end of OL treatment was the date of EOT-OL visit.

(ii) Concomitant Therapy During the Study

Study treatment-concomitant PAH-specific therapies were the same as study-concomitant PAH-specific therapies at baseline. The proportion of patients who received at least one study-concomitant PAH-specific therapy (i.e., a PAH-specific therapy ongoing or initiated after study start) was 69.4%, with an oral PDE-5 inhibitor reported for all these patients. See, Table 17.

TABLE 17
Study concomitant PAH-specific therapies, Full analysis set

| | Macitentan | | |
|---|---|---|---|
| | 10 mg | Placebo | Total |
| Specific therapy | N = 43 | N = 42 | N = 85 |
| Preferred Term | n (%) | n (%) | n (%) |
| Subjects with at least one | 30 (69.8) | 29 (69.0) | 59 (69.4) |
| specific PAH therapy | | | |
| Prostanoid | 5 (11.6) | 1 (2.4) | 6 (7.1) |
| TREPROSTINIL SODIUM | 2 (4.7) | 1 (2.4) | 3 (3.5) |
| ILOPROST | 2 (4.7) | 0 | 2 (2.4) |
| SELEXIPAG | 1 (2.3) | 0 | 1 (1.2) |
| ERA | 1 (2.3) | 0 | 1 (1.2) |
| MACITENTAN | 1 (2.3) | 0 | 1 (1.2) |
| Oral PDE5i | 30 (69.8) | 29 (69.0) | 59 (69.4) |
| SILDENAFIL | 10 (23.3) | 11 (26.2) | 21 (24.7) |
| SILDENAFIL CITRATE | 10 (23.3) | 10 (23.8) | 20 (23.5) |
| TADALAFIL | 10 (23.3) | 9 (21.4) | 19 (22.4) |

PAH advanced therapy covers oral or inhaled prostanoids, endothelin receptor antagonists, PDE-5 inhibitors, and soluble guanylate cyclase stimulators.

B. Adverse Events

An overview of the AEs reported in the DB phase of the study is shown in Tables 18 and 19 for the patients treated with macitentan in the DB and/or OL phases (macitentan DB+OL phase).

TABLE 18
Overview of treatment-emergent AEs in
the DB phase, Safety set

| | Macitentan | |
|---|---|---|
| | 10 mg | Placebo |
| | N = 43 | N = 42 |
| Characteristic | n (%) | n (%) |
| Subjects with at least one | 36 (83.7) | 33 (78.6) |
| AE | | |
| Severe AE | 7 (16.3) | 3 (7.1) |
| Drug-Related AE | 19 (44.2) | 12 (28.6) |
| AE leading to study drug | 4 (9.3) | 0 |
| discontinuation | | |
| Serious AE | 9 (20.9) | 6 (14.3) |
| Drug-Related serious AE | 5 (11.6) | 0 |
| Fatal serious AE | 0 | 1 (2.4) |

Safety set is reported on double-blind period which was defined as from first intake of study treatment until end of double-blind treatment+30 days or end of double-blind treatment for patients entering OL.

TABLE 19
Overview of treatment-emergent AEs in the macitentan
DB + OL phase, Macitentan treated set

| | Total |
|---|---|
| | N = 84 |
| Characteristic | n (%) |
| Subjects with at least one | 74 (88.1) |
| AE | |
| Severe AE | 19 (22.6) |
| Drug-Related AE | 33 (39.3) |
| AE leading to study drug | 10 (11.9) |
| discontinuation | |
| Serious AE | 25 (29.8) |
| Drug-Related serious AE | 8 (9.5) |
| Fatal serious AE | 4 (4.8) |

Macitentan treated set period was defined as from first intake of Macitentan treatment (DB or OL) up to end of Macitentan treatment (DB or OL) + 30 days. For patients entering in OL extension, the last date considered was EOT-OL.

(i) Frequency of Adverse Events

During the DB phase, the AEs that occurred more frequently in the macitentan group than in the placebo group were peripheral edema/peripheral swelling, bronchitis and decreased hemoglobin/anemia. See, Table 20. The greater frequency of AEs in the macitentan group than in the placebo group for the infections SOC was due to a higher proportion of bronchitis and AEs associated with upper respiratory tract infections. In the general disorders SOC, the higher frequency on macitentan was mainly due to edema AEs. Headache was a frequently reported event in both groups and although there was a higher frequency of nervous system disorder AEs in the macitentan group, no clear pattern or clustering of events was apparent.

TABLE 20
Treatment-emergent AEs by preferred term (at least 2
patients in either group) in the DB phase, Safety set

| | Macitentan | |
|---|---|---|
| | 10 mg | Placebo |
| | N = 43 | N = 42 |
| Preferred Term | n (%) | n (%) |
| Subjects with at least one AE | 36 (83.7) | 33 (78.6) |
| Oedema peripheral | 11 (25.6) | 5 (11.9) |
| Headache | 7 (16.3) | 7 (16.7) |
| Bronchitis | 4 (9.3) | 0 |
| Haemoglobin decreased | 3 (7.0) | 0 |
| Right ventricular failure | 3 (7.0) | 1 (2.4) |
| Abdominal pain | 2 (4.7) | 1 (2.4) |
| Acute kidney injury | 2 (4.7) | 1 (2.4) |
| Anaemia | 2 (4.7) | 0 |
| Back pain | 2 (4.7) | 1 (2.4) |
| Dizziness | 2 (4.7) | 2 (4.8) |
| Fall | 2 (4.7) | 0 |
| Hepatic encephalopathy | 2 (4.7) | 0 |
| Hypokalaemia | 2 (4.7) | 6 (14.3) |
| Hypotension | 2 (4.7) | 0 |
| Pain in extremity | 2 (4.7) | 3 (7.1) |
| Peripheral swelling | 2 (4.7) | 0 |
| Presyncope | 2 (4.7) | 0 |
| Rhinitis | 2 (4.7) | 0 |
| Tachycardia | 2 (4.7) | 0 |
| Urinary tract infection | 2 (4.7) | 0 |
| Viral upper respiratory tract infection | 2 (4.7) | 1 (2.4) |
| Diarrhoea | 1 (2.3) | 4 (9.5) |
| Dry mouth | 1 (2.3) | 2 (4.8) |
| Dyspnoea | 1 (2.3) | 2 (4.8) |
| Foot fracture | 1 (2.3) | 2 (4.8) |
| Hepatocellular carcinoma | 1 (2.3) | 2 (4.8) |
| Nausea | 1 (2.3) | 2 (4.8) |
| Cough | 0 | 3 (7.1) |
| Muscle spasms | 0 | 5 (11.9) |

Frequencies represent the number of patients with the event. Preferred Terms are based on MedDRA version 20.0. Safety set is reported on double-blind period which was defined as from first intake of study treatment until end of double-blind treatment + 30 days or end of double-blind treatment for patients entering OL.

During the macitentan DB+OL phase, the pattern of AEs that occurred most frequently was similar to that in the DB macitentan-treated patients, although the frequency of anemia/decreased hemoglobin was higher than in the DB phase.

TABLE 21
Treatment-emergent AEs by preferred term (at least 3 patients)
in the macitentan DB + OL phase, Macitentan treated set

| | Total |
|---|---|
| | N = 84 |
| Preferred Term | n (%) |
| Subjects with at least one AE | 74 (88.1) |
| Oedema peripheral | 23 (27.4) |
| Headache | 16 (19.0) |
| Anaemia | 12 (14.3) |
| Bronchitis | 8 (9.5) |
| Dizziness | 7 (8.3) |
| Pain in extremity | 7 (8.3) |
| Haemoglobin decreased | 6 (7.1) |
| Right ventricular failure | 6 (7.1) |
| Rhinitis | 5 (6.0) |
| Abdominal pain | 4 (4.8) |
| Ascites | 4 (4.8) |
| Asthenia | 4 (4.8) |
| Dyspnoea | 4 (4.8) |
| Hepatic encephalopathy | 4 (4.8) |
| Hypotension | 4 (4.8) |
| Nausea | 4 (4.8) |
| Viral upper respiratory tract infection | 4 (4.8) |
| Acute kidney injury | 3 (3.6) |
| Arthralgia | 3 (3.6) |
| Dyspnoea exertional | 3 (3.6) |
| Fall | 3 (3.6) |
| Hypokalaemia | 3 (3.6) |
| Influenza like illness | 3 (3.6) |
| Iron deficiency anaemia | 3 (3.6) |
| Muscle spasms | 3 (3.6) |
| Pruritus | 3 (3.6) |
| Pulmonary arterial hypertension | 3 (3.6) |
| Rash | 3 (3.6) |
| Urinary tract infection | 3 (3.6) |
| Vomiting | 3 (3.6) |

Frequencies represent the number of patients with the event. Preferred Terms are based on MedDRA version 20.0. Macitentan treated set period was defined as from first intake of Macitentan treatment (DB or OL) up to end of Macitentan treatment (DB or OL) + 30 days. For patients entering in OL extension, the last date considered was EOT-OL.

(ii) Intensity of Adverse Events

In the DB phase, the majority of AEs in both groups were of mild or moderate intensity. Severe AEs were reported for 16.3% (7 patients) in the macitentan group and 7.1% (3 patients in the placebo group). None of the severe AE PTs were reported for more than 2 patients, therefore no clear pattern could be identified. In the macitentan DB+OL phase, 22.6% (19 patients) had AEs of severe intensity. The most frequently reported severe AEs were peripheral edema, right ventricular failure and acute kidney injury, each of which was reported for 3.6% (3 patients).

(iii) Relationship of Adverse Events to Study Treatment

In the DB phase, the AEs that were most frequently considered to be related to study treatment by the investigator were headache and peripheral edema. Headache was reported for 16.3% (7 patients) in the macitentan group and 11.9% (5 patients) in the placebo group, and peripheral edema was reported for 9.3% (4 patients) and 2.4% (1 patient) in the placebo group. None of the other AEs that were attributed to study treatment were reported for more than 2 patients, therefore no clear pattern could be identified. In the macitentan DB+OL phase, the AEs that were most frequently considered to be related to study treatment by the investigator were headache (13.1%, 11 patients), peripheral edema (11.9%, 10 patients), anemia (4.8%, 4 patients), and decreased hemoglobin (3.6%, 3 patients). None of the other AEs that were attributed to study treatment were reported for more than 2 patients. Edema/fluid retention, anemia and headache are all listed in the prescribing information as adverse drug reactions (ADRs). AESIs are presented in Table 22 for the DB phase of the study and in Table 22 for the macitentan DB+OL phase.

TABLE 22
Treatment-emergent AEs of special interest by
preferred term in the DB phase, Safety set

| | Macitentan | | | |
|---|---|---|---|---|
| | 10 mg | | Placebo | |
| AE of special interest | N = 43 | | N = 42 | |
| Preferred Term | n (%) | | n (%) | |
| Edema and fluid retention | | | | |
| Subjects with at least one AE | 13 | (30.2) | 6 | (14.3) |
| Oedema peripheral | 11 | (25.6) | 5 | (11.9) |
| Peripheral swelling | 2 | (4.7) | 0 | |
| Localised oedema | 1 | (2.3) | 0 | |
| Fluid overload | 1 | (2.3) | 0 | |
| Joint swelling | 1 | (2.3) | 1 | (2.4) |
| Anemia | | | | |
| Subjects with at least one AE | 6 | (14.0) | 1 | (2.4) |
| Anaemia | 2 | (4.7) | 0 | |
| Iron deficiency anaemia | 1 | (2.3) | 0 | |
| Pancytopenia | 0 | | 1 | (2.4) |
| Haemoglobin decreased | 3 | (7.0) | 0 | |
| Hepatic disorders | | | | |
| Subjects with at least one AE | 6 | (14.0) | 4 | (9.5) |
| Ammonia increased | 1 | (2.3) | 0 | |
| Liver function test abnormal | 1 | (2.3) | 0 | |
| Total bile acids increased | 1 | (2.3) | 0 | |
| Blood bilirubin increased | 0 | | 1 | (2.4) |
| Gamma-glutamyltransferase | 0 | | 1 | (2.4) |
| increased | | | | |
| Ascites | 1 | (2.3) | 0 | |
| Varices oesophageal | 1 | (2.3) | 0 | |
| Hepatic encephalopathy | 2 | (4.7) | 0 | |
| Jaundice | 1 | (2.3) | 0 | |
| Hepatocellular carcinoma | 1 | (2.3) | 2 | (4.8) |

Frequencies represent the number of patients with the event. Preferred Terms are based on MedDRA version 20.0. Safety set is reported on double-blind period which was defined as from first intake of study treatment until end of double-blind treatment + 30 days or end of double-blind treatment for patients entering OL.

TABLE 23
Treatment-emergent AEs of special interest by preferred term
in the macitentan DB + OL phase, Macitentan treated set

| | Total |
|---|---|
| AEs of special interest | N = 84 |
| Preferred Term | n (%) |
| Edema and fluid retention | |
| Subjects with atleast one AE | 26 (31.0) |
| Oedema peripheral | 23 (27.4) |
| Peripheral swelling | 2 (2.4) |
| Generalised oedema | 1 (1.2) |
| Localised oedema | 1 (1.2) |
| Swelling | 1 (1.2) |
| Fluid overload | 1 (1.2) |
| Fluid retention | 1 (1.2) |
| Joint swelling | 1 (1.2) |
| Anemia | |
| Subjects with atleast one AE | 22 (26.2) |
| Anaemia | 12 (14.3) |
| Iron deficiency anaemia | 3 (3.6) |
| Haemoglobin decreased | 6 (7.1) |
| Haematocrit decreased | 1 (1.2) |
| Anaemia postoperative | 1 (1.2) |
| Hepatic disorders | |
| Subjects with atleast one AE | 13 (15.5) |
| Ascites | 4 (4.8) |
| Portal hypertensive gastropathy | 1 (1.2) |
| Varices oesophageal | 1 (1.2) |
| Ammonia increased | 2 (2.4) |
| Total bile acids increased | 2 (2.4) |
| Blood bilirubin increased | 1 (1.2) |
| Liver function test abnormal | 1 (1.2) |
| Liver function test increased | 1 (1.2) |
| Hepatic encephalopathy | 4 (4.8) |
| Jaundice | 1 (1.2) |
| Hepatocellular carcinoma | 1 (1.2) |
| Portopulmonary hypertension | 1 (1.2) |

Frequencies represent the number of patients with the event. Preferred Terms are based on MedDRA version 20.0. Macitentan treated set period was defined as from first intake of Macitentan treatment (DB or OL) up to end of Macitentan treatment (DB or OL) + 30 days. For patients entering in OL extension, the last date considered was EOTOL.

Edema and fluid retention: In the DB phase, edema and fluid retention AEs were reported for 30.2% patients in the macitentan group vs 14.3% in the placebo group. The majority of these events were peripheral edema. In the macitentan group, one patient had 3 SAEs (1 event each of peripheral edema, localized edema and fluid overload) but none led to discontinuation of study treatment. See, Tables 26 and 28. In the placebo group, none of the AEs were serious or led to discontinuation of study treatment. In the macitentan DB+OL phase, 31.0% of patients had edema and fluid retention AEs. See, Table 23. As for the DB phase, the majority of these events were peripheral edema. 2 of the patients had AEs that were reported as serious (1 SAE of peripheral edema and 3 SAEs of peripheral edema, localized edema and fluid overload as described above). One of the SAEs and the non-serious AE of peripheral edema resulted in discontinuation of study treatment. See, Tables 27 and 29.

Anemia: In the DB phase, anemia AEs were reported for 14.0% patients in the macitentan group vs 2.4% in the placebo group. None of the AEs were reported as SAEs, but 1 case in the macitentan group led to discontinuation of study treatment. See, Tables 26 and 28. None of the cases of anemia were treated with blood transfusion. Laboratory data showed that in the macitentan group 27.9% of patients had hemoglobin decreases of ≥20 g/L vs 4.9% in the placebo group. See, Table 24. In the macitentan group, 1 of these 12 patients had a decrease of ≥50 g/L. In 3 patients in the macitentan group and 2 patients in the placebo group, hemoglobin decreased to concentrations of ≤100 g/L, but none decreased to ≤80 g/L. In the macitentan DB+OL phase, 26.2% of patients had anemia AEs. 2 of the AEs were reported as SAEs during the core study (1 case of anemia and 1 case of iron deficiency anemia). See, Table 28. In addition, a third subject had a non-serious anemia AE in the core study that was reported as an SAE when the patient was hospitalized due to anemia, one week after entering the OLE (the patient had a medical history of anemia with transfusion, bleeding and an enlarged spleen). Two AEs of anemia resulted in discontinuation of study treatment. See, Table 30. Laboratory data showed that 37.3% of patients in the macitentan DB+OL phase had hemoglobin decreases of ≥20 g/L. See, Table 25. 6 of these 31 patients had a decrease of ≥50 g/L. In 13 patients, there were decreases in hemoglobin concentrations to ≤100 g/L, with 4 patients having decreases to ≤80 g/L. One patient with a hemoglobin decrease of ≥50 g/L that resulted in a value of 53 g/L was treated with a blood transfusion (reported as an SAE and treatment was discontinued).

TABLE 24
Liver and hemoglobin laboratory abnormalities
in the DB phase, Safety set

| | Macitentan | |
|---|---|---|
| | 10 mg | |
| Abnormality | N = 43 | N = 42 |
| Placebo | n/Nn (%) | n/Nn (%) |
| ALT or AST >= 3*ULN | 1/43 (2.3) | 0/41 |
| ALT or AST >= 5*ULN | 0/43 | 0/41 |
| ALT or AST >= 8*ULN | 0/43 | 0/41 |
| ALT or AST >= 3*ULN and <5*ULN | 1/43 (2.3) | 0/41 |
| ALT or AST >= 5*ULN and <8*ULN | 0/43 | 0/41 |
| ALT or AST >= 3*ULN + total | 1/43 (2.3) | 0/41 |
| bilirubin >= 2*ULN (a) | | |
| ALT or AST >= 3*ULN + total | 1/43 (2.3) | 0/41 |
| bilirubin >= 2*ULN | | |
| (and increased as compared to baseline) (a) | | |
| Hemoglobin <= 80 g/L | 0/43 | 0/41 |
| Hemoglobin > 80 g/L and <=100 g/L | 3/43 (7.0) | 2/41 (4.9) |
| Hemoglobin decrease from baseline >= 20 g/L | 11/43 (25.6) | 2/41 (4.9) |
| and <50 g/L | | |
| Hemoglobin decrease from baseline >= 50 g/L | 1/43 (2.3) | 0/41 |
| Hemoglobin < 100 g/L and decrease from | 1/43 (2.3) | 1/41 (2.4) |
| baseline >= 20 g/L (a) | | |

(a) At the same time. n is the number of patients who responded. N is the total number of patients in the treatment group. Nn is the number of patients at risk with non-missing values. Frequencies present the number of patients with defined abnormality reported on the most extreme post-baseline values during the specified period. Safety set is reported on double-blind period which was defined as from first intake of study treatment until end of double-blind treatment + 30 days or end of double-blind treatment for patients entering OL.

TABLE 25
Liver and hemoglobin laboratory abnormalities in the macitentan
DB + OL phase, Macitentan treated set

| | Total |
|---|---|
| | N = 84 |
| Abnormality | n/Nn (%) |
| ALT or AST >= 3*ULN | 2/84 (2.4) |
| ALT or AST >= 5*ULN | 1/84 (1.2) |
| ALT or AST >= 8*ULN | 1/84 (1.2) |
| ALT or AST >= 3*ULN and <5*ULN | 1/84 (1.2) |
| ALT or AST >= 5*ULN and <8*ULN | 0/84 |
| ALT or AST >= 3*ULN + total bilirubin >= 2*ULN (a) | 2/84 (2.4) |
| ALT or AST >= 3*ULN + total bilirubin >= 2*ULN | 2/84 (2.4) |
| (and increased as compared to baseline) (a) | |
| Hemoglobin <= 80 g/L | 4/83 (4.8) |
| Hemoglobin > 80 g/L and <=100 g/L | 9/83 (10.8) |
| Hemoglobin decrease from baseline >= 20 g/L and <50 g/L | 25/83 (30.1) |
| Hemoglobin decrease from baseline >= 50 g/L | 6/83 (7.2) |
| Hemoglobin < 100 g/L and decrease from | 9/83 (10.8) |
| baseline >= 20 g/L (a) | |

(a) At the same time. n is the number of patients who responded. N is the total number of patients in the treatment group. Nn is the number of patients at risk with non-missing values. Frequencies present the number of patients with defined abnormality reported on the most extreme post-baseline values during the specified period. Macitentan treated set period was defined as from first intake of Macitentan treatment (DB or OL) up to end of Macitentan treatment (DB or OL) + 30 days. For patients entering in OL extension, the last date considered was EOT-OL.

Hepatic disorders: In the DB phase, hepatic disorder AEs were reported for 14.0% patients in the macitentan group vs 9.5% in the placebo group. In the macitentan group, 3 patients had SAEs (1 case each of ascites, hepatic encephalopathy and hepatocellular carcinoma). The patient with ascites was subsequently re-hospitalized 6 weeks after EOT for ascites and acute liver failure, which the investigator assessed as related to study treatment and reported as a SUSAR. In the placebo group, there were 2 cases of hepatocellular carcinoma. See, Table 27. None of the events resulted in discontinuation of study treatment. Laboratory data showed that 1 macitentan-treated patient in the DB phase had an aminotransferase increase to >3×ULN (ALT: 3.1×ULN, AST: 3.2×ULN); this was associated with an increase in bilirubin to >2×ULN (2.7×ULN). See, Table 24.. In the macitentan DB+OL phase, 15.5% of patients had hepatic disorder AEs. 5 of the patients had AEs that were reported as SAEs. In 2 of these cases, the events were associated with hepatocellular carcinoma. For one of these patients, hepatocellular carcinoma was initially reported during DB placebo treatment, with SAEs of malignant ascites, hepatic encephalopathy and increased liver function test reported during OL macitentan treatment. The patient discontinued treatment due to increased liver function test and subsequently died due to the hepatocellular carcinoma. In the second case, it was suspected early in the study that the patient had a hepatocellular carcinoma, with investigations commencing on Day of macitentan treatment, with lesions consistent with a carcinoma identified on Day 60. The patient subsequently had an SAE of hepatic encephalopathy. Two patients had SAEs of ascites and one patient had an SAE of hepatic encephalopathy. Laboratory data showed that 2 patients in the macitentan DB+OL phase had an aminotransferase increase to >3×ULN. See, Table 25. One of these was the patient described above with elevations during the DB phase and an additional patient with ALT=3.6×ULN, AST: 8.6×ULN, bilirubin: 11.1×ULN, in the context of malignant ascites and hepatic encelophopathy. During the DB phase, none of the patients in the macitentan group (n=23) had a worsening in Child-Pugh category. One patient in the placebo group (n=25) worsened from category A at baseline to category B at Week 12. In the combined DB+OL phase, 1 patient who had received placebo in the DB phase worsened from category A to B during macitentan treatment. At baseline, the median MELD score in both groups was 10, ranging from 6-17 in the macitentan 10 mg group (n=31) and 6-18 in the placebo group (n=27). At Week 12, the results were consistent, with a median of 10, ranging from 6-15 in both groups. In the combined DB+OL phase the results were consistent for patients treated with macitentan for 12 weeks (including those who switched from DB placebo) and 24 weeks.

C. Deaths, Other Serious Adverse Events, and Other Significant Adverse Events

(i) Deaths

There were no deaths during the DB phase of the study. During the OL phase, 4 patients died as shown in Table 26. Two of the deaths (worsening PAH and pneumonia) were consistent with complications associated with PAH. One patient with a history of hepatic cirrhosis and diabetes died due to a subdural hematoma. The hepatocellular carcinoma case was initially reported in the DB phase in a placebo-treated patient (with a history of the condition). An additional death was reported 36 days after EOT for the OL period (for this reason the death is not included in the summaries for the macitentan DB+OL phase) due to an SAE of acute kidney injury. The patient had been in the DB placebo group and had switched to macitentan 18 days prior to the onset of events that ultimately resulted in death. It was reported that on an unspecified date prior to the events, the patient had stopped taking diuretics and had not adhered to a low sodium diet. There were 2 additional deaths during the OLE. One of these was due to unknown causes (the patient died at home) and the second was due to hemorrhagic stroke, which was reported as resulting from a head injury due to a fall. None of the deaths were considered by the investigator to be related to macitentan treatment.

TABLE 26
Cause of death in the OL phase, Macitentan treated set

| | Total N = 84 |
|---|---|
| | n (%) |
| Total deaths | 4 (4.8) |
| Reason for death | |
| Hepatocellular Carcinoma | 1 (1.2) |
| Pneumonia | 1 (1.2) |
| Pulmonary Arterial Hypertension | 1 (1.2) |
| Subdural Haematoma | 1 (1.2) |

Macitentan treated set period was defined as from first intake of Macitentan treatment (DB or OL) up to end of Macitentan treatment + 30 days.

(ii) Serious Adverse Events

In the DB phase, the proportion of patients who had SAEs was 20.9% in the macitentan group vs 14.3% in the placebo group. See, Table 27. Almost all of the events were reported in individual patients and there was no particular pattern or clustering of events in either group. In the macitentan DB+OL phase, 29.8% of patients had an SAE. See, Table 28. The most frequently reported SAEs were right ventricular failure and hepatic encephalopathy, both of which are complications of the underlying PoPH.

TABLE 27
Treatment-emergent SAEs by preferred term in the DB phase, Safety set

| | Macitentan | |
|---|---|---|
| | 10 mg | Placebo |
| | N = 43 | N = 42 |
| Preferred Term | n (%) | n (%) |
| Subjects with at least one SAE | 9 (20.9) | 6 (14.3) |
| Right ventricular failure | 2 (4.7) | 1 (2.4) |
| Acute kidney injury | 1 (2.3) | 1 (2.4) |
| Alveolitis | 1 (2.3) | 0 |
| Ascites | 1 (2.3) | 0 |
| Asthma | 1 (2.3) | 0 |
| Atrial fibrillation | 1 (2.3) | 0 |
| Fluid overload | 1 (2.3) | 0 |
| Haemoptysis | 1 (2.3) | 0 |
| Hepatic encephalopathy | 1 (2.3) | 0 |
| Hepatocellular carcinoma | 1 (2.3) | 2 (4.8) |
| Hypersensitivity | 1 (2.3) | 0 |
| Ileus | 1 (2.3) | 0 |
| Left ventricular failure | 1 (2.3) | 0 |
| Localised oedema | 1 (2.3) | 0 |
| Lung infection | 1 (2.3) | 0 |
| Oedema peripheral | 1 (2.3) | 0 |
| Presyncope | 1 (2.3) | 0 |
| Pulmonary arterial hypertension | 1 (2.3) | 0 |
| Pulmonary toxicity | 1 (2.3) | 0 |
| Syncope | 1 (2.3) | 0 |
| Troponin I increased | 1 (2.3) | 0 |
| Urosepsis | 1 (2.3) | 0 |
| Aneurysm repair | 0 | 1 (2.4) |
| Localised infection | 0 | 1 (2.4) |
| Vascular procedure complication | 0 | 1 (2.4) |

Frequencies represent the number of patients with the event. Preferred Terms are based on MedDRA version 20.0. Safety set is reported on double-blind period which was defined as from first intake of study treatment until end of double-blind treatment + 30 days or end of double-blind treatment for patients entering OL.

TABLE 28
Treatment-emergent SAEs in the macitentan DB + OL
phase by preferred term, Macitentan treated set

| | N = 84 |
|---|---|
| Preferred Term Total | n(%) |
| Subjects with at least one SAE | 25 (29.8) |
| Right ventricular failure | 4 (4.8) |
| Hepatic encephalopathy | 3 (3.6) |
| Acute kidney injury | 2 (2.4) |
| Anaemia | 2 (2.4) |
| Ascites | 2 (2.4) |
| Melaena | 2 (2.4) |
| Oedema peripheral | 2 (2.4) |
| Pulmonary arterial hypertension | 2 (2.4) |
| Abdominal pain | 1 (1.2) |
| Alveolitis | 1 (1.2) |
| Asthma | 1 (1.2) |
| Atrial fibrillation | 1 (1.2) |
| Bronchitis | 1 (1.2) |
| Chronic kidney disease | 1 (1.2) |
| Fall | 1 (1.2) |
| Fluid overload | 1 (1.2) |
| Gastrointestinal haemorrhage | 1 (1.2) |
| Haemoptysis | 1 (1.2) |
| Hepatocellular carcinoma | 1 (1.2) |
| Humerus fracture | 1 (1.2) |
| Hypersensitivity | 1 (1.2) |
| Ileus | 1 (1.2) |
| Intervertebral disc protrusion | 1 (1.2) |
| Iron deficiency anaemia | 1 (1.2) |
| Left ventricular failure | 1 (1.2) |
| Liver function test increased | 1 (1.2) |
| Localised oedema | 1 (1.2) |
| Lung infection | 1 (1.2) |
| Malignant ascites | 1 (1.2) |
| Osteitis | 1 (1.2) |
| Pneumonia | 1 (1.2) |
| Presyncope | 1 (1.2) |
| Priapism | 1 (1.2) |
| Pulmonary toxicity | 1 (1.2) |
| Staphylococcal infection | 1 (1.2) |
| Subdural haematoma | 1 (1.2) |
| Syncope | 1 (1.2) |
| Thrombocytopenia | 1 (1.2) |
| Tinnitus | 1 (1.2) |
| Troponin I increased | 1 (1.2) |
| Urosepsis | 1 (1.2) |

Frequencies represent the number of patients with the event. Macitentan treated set period was defined as from first intake of Macitentan treatment (DB or OL) up to end of Macitentan treatment (DB or OL) + 30 days. For patients entering in OL extension, the last date considered was EOT-OL.

(iii) Adverse Events Leading to Discontinuation of Study Treatment

In the DB phase, 4 patients, all in the macitentan group, discontinued treatment due to AEs. See, Table 29. All of the events were reported in individual patients. In the macitentan DB+OL phase, 10 patients discontinued treatment due to AEs. See, Table 30. Anemia and peripheral edema each led to the discontinuation of treatment for 2 patients. All of the other events were reported in individual patients.

TABLE 29
Adverse events leading to premature discontinuation of study
treatment in the DB phase by preferred term, Safety set

| | Macitentan | |
|---|---|---|
| | 10 mg | Placebo |
| | N = 43 | N = 42 |
| Preferred Term | n (%) | n (%) |
| Subjects with at least one AE | 4 (9.3) | 0 |
| Alveolitis | 1 (2.3) | 0 |
| Anaemia | 1 (2.3) | 0 |
| Hypersensitivity | 1 (2.3) | 0 |
| Pulmonary arterial hypertension | 1 (2.3) | 0 |

Frequencies represent the number of patients with the event. Safety set is reported on double-blind period which was defined as from first intake of study treatment until end of double-blind treatment + 30 days or end of double-blind treatment for patients entering OL.

TABLE 30
AEs leading to premature discontinuation of study treatment in the
macitentan DB + OL phase by preferred term, Macitentan treated set

| | Total |
|---|---|
| System Organ Class | N = 84 |
| Preferred Term | n(%) |
| Subjects with at least one AE | 10 (11.9) |
| Anaemia | 2 (2.4) |
| Oedema peripheral | 2 (2.4) |
| Alveolitis | 1 (1.2) |
| Ascites | 1 (1.2) |
| Diarrhoea | 1 (1.2) |
| Hypersensitivity | 1 (1.2) |
| Liver function test increased | 1 (1.2) |
| Pulmonary arterial hypertension | 1 (1.2) |
| Pneumonia | 1 (1.2) |

Frequencies represent the number of patients with the event. Macitentan treated set period was defined as from first intake of Macitentan treatment (DB or OL) up to end of Macitentan treatment (DB or OL) + 30 days. For patients entering in OL extension, the last date considered was OTOL.

D. Hematology

In the DB phase, 2/29 patients in the macitentan group and 2/32 patients in the placebo group who had normal platelets at baseline had decreases to <75×10⁹/L. In the macitentan DB+OL phase, 7 macitentan-treated patients had decreases from normal at macitentan baseline to <75×10⁹/L. Platelet concentrations over time were highly variable, and in the DB phase, the median decrease in the macitentan and placebo groups at Week 12 were 11.0×10⁹/L and 2.8×10⁹/L, respectively. In the combined DB+OL phase, there was no median decrease from baseline in platelet counts. Decreases in white blood cell counts were observed during the study, particularly with respect to lymphocytes and neutrophils, as shown in Table 31. At any time during the DB phase, 9/38 patients in the macitentan group had shifts in lymphocyte counts from normal at baseline to <0.8×10⁹/L, compared to 2/33 in the placebo group. Median lymphocyte count decreased from 1.21 (range: 0.54-3.67)×10⁹/L at baseline to 1.04 (range: 0.41-3.40)×10⁹/L at Week 12, representing a median decrease of 0.28×10⁹/L. No decrease in median lymphocyte count was observed in the placebo group.

In the macitentan DB+OL phase, the proportion of patients with shifts in lymphocyte counts from normal at baseline to <0.8×10⁹/L was 21/72. See, Table 32. Median lymphocyte count decreased from 1.22 (range: 0.47-3.67)×10⁹/L at baseline to 1.05 (range: 0.36-3.99)×10⁹/L at Week 24, representing a median decrease of 0.24×10⁹/L. For neutrophil count in the DB phase, 6/41 patients in the macitentan group had decreases from normal at baseline to <1.5×10⁹/L vs 3/41 in the placebo group. See, Table 31. Median neutrophil count decreased from 3.33 (range: 1.13-6.43)×10⁹/L at baseline to 2.25 (range: 0.54-6.71)×10⁹/L at Week 12, representing a median decrease of 0.72×10⁹/L. In the placebo group, there was a median decrease of 0.11×10⁹/L from a median baseline of 2.91 (range: 1.22-8.04)×10⁹/L. In the macitentan DB+OL phase, the proportion of patients with shifts in neutrophil counts from normal at baseline to notably low was 17/81. See, Table 32. Median neutrophil count decreased from 3.33 (range: 1.13-6.43)×10⁹/L at baseline to 2.40 (range: 0.68-4.56)×10⁹/L at Week 24, representing a median decrease of 0.33×10⁹/L.

TABLE 31
Hematology: Worst notably low, treatment-emergent white
blood cell count abnormalities in the DB phase, Safety set

| | Normal at baseline | | LL at baseline | |
|---|---|---|---|---|
| Treatment-emergent | Macitentan | | Macitentan | |
| Marked | 10 mg | Placebo | 10 mg | Placebo |
| Abnormality | n/Nn | n/Nn | n/Nn | n/Nn |
| Leukocytes (10⁹/L) | | | | |
| <3.0 −≥ 2.0 (LL) | 5/38 | 2/37 | — | — |
| <2.0 (LLL) | 0/38 | 1/37 | 2/5 | 1/5 |
| Missing post-baseline | 0/38 | 1/37 | 0/5 | 0/5 |
| Lymphocytes (10⁹/L) | | | | |
| <0.8 −≥ 0.5 (LL) | 8/38 | 2/33 | — | — |
| <0.5 (LLL) | 1/38 | 0/33 | 3/5 | 1/9 |
| Missing post-baseline | 0/38 | 1/33 | 0/5 | 0/9 |
| Neutrophils (10⁹/L) | | | | |
| <1.5 −≥ 1.0 (LL) | 4/41 | 2/41 | — | — |
| <1.0 (LLL) | 2/41 | 1/41 | 0/2 | 0/1 |
| Missing post-baseline | 0/41 | 1/41 | 0/2 | 0/1 |

n is the number of patients with an abnormally low value. N is the total number of patients in the treatment group. Nn is the number of patients at risk with non-missing values. Safety set is reported on double-blind period which was defined as from first intake of study treatment until end of double-blind treatment + 30 days or end of double-blind treatment for patients entering OL.

TABLE 32
Hematology: Worst notably low, treatment-emergent white
blood cell count abnormalities in the macitentan
DB + OL phase, Macitentan treated set

| Treatment-emergent Marked | Normal at baseline | LL at baseline |
|---|---|---|
| Abnormality | n/Nn (%) | n/Nn (%) |
| Leukocytes (10⁹/L) | | |
| <3.0 −≥ 2.0 (LL) | 9/76 | — |
| <2.0 (LLL) | 4/76 | 3/7 |
| Missing post-baseline | 1/76 | 0/7 |
| Lymphocytes (10⁹/L) | | |
| <0.8 −≥ 0.5 (LL) | 19/72 | — |
| <0.5 (LLL) | 2/72 | 6/10 |
| Missing post-baseline | 0/72 | 1/10 |
| Neutrophils (10⁹/L) | | |
| <1.5 −≥ 1.0 (LL) | 12/81 | — |
| <1.0 (LLL) | 5/81 | 0/2 |
| Missing post-baseline | 1/81 | 0/2 |

n is the number of patients with an abnormally low value. N is the total number of patients in the treatment group. Nn is the number of patients at risk with non-missing values. Macitentan treated set period was defined as from first intake of Macitentan treatment (DB or OL) up to end of Macitentan treatment (DB or OL) + 30 days. For patients entering in OL extension, the last date considered was EOT-OL.inghu

Summary and Conclusions

A. Efficacy Summary

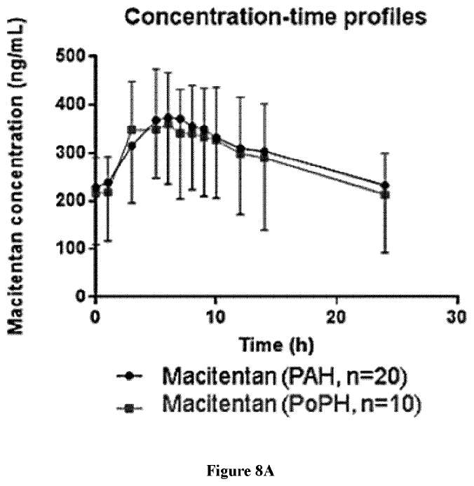

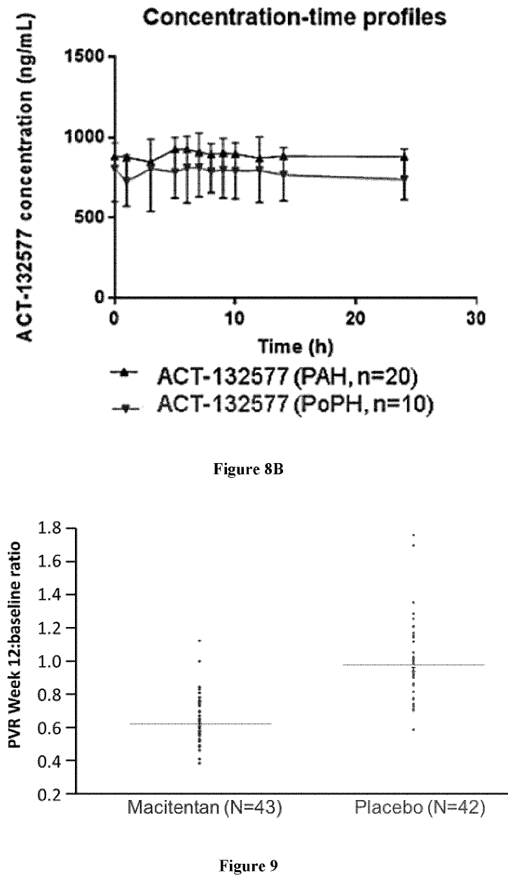

In the main analysis of the primary endpoint using the FAS (N=85), the geometric mean (95% CLs) ratio of Week 12 to baseline PVR was 0.63 (0.58, 0.67) and 0.98 (0.91, 1.05) in the macitentan and placebo groups, respectively. The log-transformed geometric ratios of PVR at Week 12 to baseline PVR were analyzed using an ANCOVA, (treatment, background PAH-specific therapy at baseline and region as factors, and log-transformed PVR at baseline as a covariate). The resultant treatment effect at Week 12 was 0.65 (95% CLs: 0.59, 0.72), p<0.0001, i.e., a 35% reduction in PVR on macitentan vs placebo. See, FIGS. 8A and 8B. Therefore, the null hypothesis was rejected as the 2-sided 95% confidence interval did not include 1.0. Mean PVR (±SD) decreased by 202.1 (±123.56) dyn·sec/cm⁵ from baseline to Week 12 in the macitentan group compared to 7.2 (±122.20) dyn·sec/cm⁵ in the placebo group. A supportive analysis using the PPS (N=73) provided consistent results with those for the FAS.

The observed effect on PVR across demographic and baseline disease characteristic pre-specified subgroups in the FAS was consistent with the overall treatment effect, with no indication of heterogeneity.

No adjustment was made for multiplicity for secondary endpoints, therefore all corresponding p-values provided are of an exploratory nature. The main analysis of the secondary 6MWD endpoint was performed using an MMRM model adjusted for treatment, visit, region, PAH-specific therapy at baseline, and treatment-by-visit interaction as factors, and baseline 6MWD and WHO FC as covariates. The LS mean difference (macitentan−placebo) of change from baseline up to Week 12 in 6MWD was 9.73 m (95% CLs: −14.5, 33.95), p=0.4264.

For the RHC secondary endpoints, there was a clinically relevant increase in cardiac index (0.52 L/min/m² [95% CLs: 0.22, 0.81]; p=0.0009) and clinically relevant decreases in mPAP (−5.99 mmHg [95% CLs: −8.40, −3.57]; p<0.0001) and TPR (−171.48 dyn·sec/cm⁵ [95% CLs: −223.67, −119.30]; p<0.0001) in the macitentan group compared to the placebo group at Week 12. No clinically relevant effect was seen for mRAP or SVO₂. Despite the relevant increase in cardiac index in the macitentan group, no increase in HVPG was observed vs placebo. The differences or ratio of the changes from baseline to Week 12 for NT-proBNP for the macitentan group vs the placebo group were not clinically relevant. No relevant change from baseline to Week 12 for WHO FC was seen in the macitentan group vs placebo.

B. Pharmacokinetic Summary

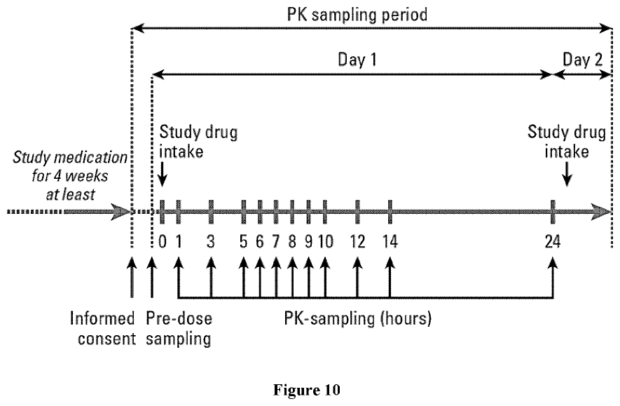

A PK substudy was performed in patients who had received OL macitentan 10 mg o.d. for at least 4 weeks. See, FIG. 10. The PKS included 10 patients. Mean±SD trough plasma concentrations of macitentan and ACT-132577 (n=8) were 213.1±86.4 ng/mL and 737.4±190.0 ng/mL, respectively. See, Table 33.

TABLE 33
PK sub-study patient characteristics and demographics at
double −= blind treatment baseline

| | Macitentan | |
|---|---|---|
| | (N = 10) | |
| Sex, male, n (%) | 7 | (70.0) |
| Age, years, mean (SD) | 54.9 | (8.6) |
| BMI, kg/m², mean (SD) | 28.75 | (4.79) |
| Baseline PAH therapy, n (%) | 6 | (60.0) |
| Baseline MELD score*, mean (SD) | 9.4 | (2.5) |
| Causes of portal hypertension, n (%) | | |
| Alcoholic cirrhosis | 5 | (50.0) |
| Autoimmune hepatitis | 2 | (20.0) |
| Hepatitis | 1 | (10.0) |
| Non-alcoholic steatohepatitis | 1 | (10.0) |
| Alcoholic cirrhosis + portal vein | 1 | (10.0) |
| thrombosis | | |

*MELD score calculated post-hoc based on the relevant available information (n = 9). BMI, body mass index; PAH, pulmonary arterial hypertension; SD, standard deviation.

Median (range) tₘₐₓ values for macitentan and ACT-132577 were 6.5 h (3.0, 10.0) and 6.5 h (0.0, 24.0), respectively. The geometric means (95% confidence intervals) for Cₘₐₓ and AUC of macitentan were 368.6 ng/mL (306.9, 442.6) and 6655.4 h*ng/mL (5229.6, 8470.0), respectively. The geometric means (95% confidence intervals) for Cₘₐₓ and AUC_{T} of ACT-132577 were 869.8 ng/mL (728.2, 1038.9) and 18100.0 h*ng/mL (14795.4, 22142.9), respectively.

The PK results indicate that exposure to macitentan and its active metabolite at steady state are comparable between patients with PoPH and other forms of PAH. See, FIGS. 8A and 8B.

C. Safety Summary

The safety set included 85 patients (43 on macitentan, 42 on placebo).

The mean duration of exposure to study treatment was approximately 12 weeks in the macitentan and placebo groups during the DB phase, and the total mean exposure on macitentan in the combined DB and OL phase was approximately 17 weeks.

The AEs that were most frequently reported at a higher incidence on macitentan than placebo were associated with the labeled ADRs of edema/fluid retention and anemia. In the DB phase, edema and fluid retention AEs were reported for 13 (30.2%) patients in the macitentan group vs 6 (14.3%) in the placebo group. The majority of these events were peripheral edema. In the macitentan group, one patient had 3 SAEs (1 event each of peripheral edema, localized edema and fluid overload) but none led to discontinuation of study treatment. In the macitentan DB+OL phase, 26 (31.0%) patients had edema and fluid retention AEs. As for the DD phase, the majority of these events were peripheral edema. 2 of the patients had AEs that were reported as serious (1 SAE of peripheral edema and 3 SAEs of peripheral edema, localized edema and fluid overload as described above). One of the SAEs and one of the non-serious AE of peripheral edema resulted in discontinuation of study treatment.

In the DB phase, anemia AEs were reported for 6 (14.0%) patients in the macitentan group vs 1 (2.4%) in the placebo group. In the macitentan DB+OL phase, anemia AEs were reported for 22 (26.2%) patients. Laboratory data showed that in the DB phase, 12 (27.9%) patients in the macitentan group had hemoglobin decreases of ≥20 g/L vs 2 (4.9%) patients in the placebo group. In the macitentan group, one of the decreases was ≥50 g/L. In 3 patients in the macitentan group and 2 patients in the placebo group, there were decreases in hemoglobin concentrations to s 100 g/L, but none were ≤80 g/L.

In the macitentan DB+OL phase, 22 (26.2%) patients had anemia AEs. 3 of the AEs were reported as SAEs (2 cases of anemia and 1 case of iron deficiency anemia). 2 patients discontinued study treatment as a result of anemia. Laboratory data showed that 31 (37.3%) patients in the macitentan DB+OL phase had hemoglobin decreases of ≥20 g/L, with 6 of these patients having a decrease of ≥50 g/L. 13 patients had decreases in hemoglobin concentrations to ≤100 g/L, with 4 patients having decreases to ≤80 g/L. One patient with a hemoglobin decrease of ≥50 g/L that resulted in a value of 53 g/L was treated with a blood transfusion (reported as an SAE and AE leading to discontinuation of treatment).

Hepatic disorder AEs were frequent in this population due to their underlying hepatic disease. In the DB phase, hepatic disorder AEs were reported for 6 (14.0%) patients in the macitentan group vs 4 (9.5%) in the placebo group. In the macitentan DB+OL phase, 13 (15.5%) patients had hepatic disorder AEs. There were 2 macitentan-treated patients with Hy's Law range liver test abnormalities, but these occurred in the context of liver conditions that were not associated with macitentan treatment.

There was no particular pattern or clustering of events that were serious or resulted in death or discontinuation of study treatment. There were no deaths during the DB phase of the study. Across the OL phase (and after the end of its follow-up) and ongoing OLE phase, 7 patients who were treated with macitentan died. The deaths were consistent with worsening of PH, comorbidities associated with the patients underlying liver disease, or other underlying disease conditions. None of the deaths were considered related to macitentan treatment by the investigator.

There was no indication of any notable effect of macitentan on vital signs, body weight or BMI.

D. Conclusions

The results indicate that macitentan has a good safety profile and is well tolerated in patients with PoPH and mild or moderate liver impairment. Specifically, macitentan treatment resulted in a clinically meaningful and statistically significant reduction in pulmonary vascular resistance of 35% vs placebo at Week 12 (p<0.0001).

In addition, for the other right heart catheterization secondary endpoints, there was a clinically relevant increase in cardiac index and clinically relevant decreases in mean pulmonary arterial pressure and total pulmonary resistance in the macitentan group compared to the placebo group at Week 12. Despite the relevant increase in cardiac index in the macitentan group vs the placebo group, no increase in hepatic venous pressure gradient was observed. Although there was a numerical increase in 6-minute walk distance in the macitentan group compared to the placebo group, the effect was not clinically relevant.

The pharmacokinetic results showed that plasma concentrations were consistent with those in other macitentan studies, such as SERAPHIN, or with other forms of PAH.

### Example 2: Liver Transplant Analyses

A. Objectives

Objectives of the analyses include:
To evaluate the effect of macitentan on N-terminal pro b-type natriuretic peptide (NT-proBNP) relative to the threshold of 300 ng/L compared to placebo in patients with PoPH.
To evaluate the effect of macitentan on liver transplant perioperative risk classification as compared to placebo in patients with PoPH.
To evaluate the effect of macitentan on MELD exception eligibility as compared to placebo in patients with PoPH.
To evaluate the effect of macitentan on liver transplant waitlist mortality risk as compared to placebo in patients with PoPH.

B. Methods

(i) Diagnosis and Main Criteria for Inclusion:

The study enrolled adult male or female patients (≥18 years) with a confirmed diagnosis of symptomatic PoPH, a baseline PVR of ≥4 WU (≥320 dyn·s·cm⁻⁵) and who were capable of performing a 6-minute walk test with a distance ≥50 m. The study allowed patients to be enrolled who were either PAH treatment naïve or were receiving background PDE-5 inhibitors, sGC stimulators, or inhaled prostanoid therapy. Patients with severe hepatic impairment, as defined by Child-Pugh class C liver disease or a MELD score ≥19 were excluded.

(ii) Criteria for Evaluation:

Post-Hoc, Exploratory Criteria:
Proportion of patients with NT-proBNP decrease from above 300 ng/L to below this threshold
Odds ratio for improvement to a more favorable liver transplant perioperative risk category
Odds ratio for becoming eligible for the MELD exception
Odds ratio for improvement to a more favorable waitlist mortality risk category

(iii) Statistical Methods:

Analyses were performed as described in an exploratory statistical analysis plan (SAP) in Example 1. Analyses were performed on full analysis set (as per randomization, N=85) and no data imputation was made. NT-proBNP was analyzed descriptively by treatment group using shift table from baseline to Week 12 with categories “<=300 ng/L”, “>300 ng/L” and “Missing”.

Risk categories related to liver transplant (transplant perioperative risk classification, MELD exception eligibility and waitlist mortality risk) were analyzed descriptively by treatment group using shift tables from baseline to Week 12 with categories as defined in Example 1. An exact logistic regression with factors for treatment and risk category at baseline was used to compute the odds ratio (macitentan vs placebo) for improvement to a better risk category (displayed with associated 95% confidence interval and p-value).

(iv) Sample Size:

Post-hoc analyses were made on the Full Analysis Set (FAS), without imputation.

C. Results

(i) Study Population

Exposure to study treatment: The median duration of DB treatment exposure was approximately 12 weeks and was similar for macitentan and placebo.

More than 90% of patients in both treatment groups received treatment up to the Week 12 visit window.

(ii) Exploratory Efficacy Results

Change in NT-proBNP

Relevance of NT-proBNP in PAH:

NT-proBNP is recognized as a marker of myocardial stress and cardiac overload that is relevant in PAH. Risk assessment is based on several parameters such as NT-proBNP, WHO FC, 6MWD, imaging and RHC. Inexpensive and objective, NT-proBNP is a key component of risk assessment in clinical practice. A result for an NT-proBNP plasma level <300 ng/L is considered as a low risk criterion, while NT-proBNP plasma level between 300 and 1400 ng/L is considered as an intermediate risk criterion and NT-proBNP plasma level >1400 ng/L is considered as a high risk criterion. Reaching an NT-proBNP plasma level <300 ng/L is therefore a relevant treatment target in PAH.

Among patients with NT-proBNP above 300 ng/L at baseline, NT-proBNP decreased to below this threshold after 12 weeks in 6 of 16 (37.5%) macitentan patients and in 1 of 11 (9.1%) placebo patients (Table 34). This suggests a favorable effect of macitentan in patients with elevated baseline NT-proBNP.

TABLE 34
Shift table from baseline to Week 12 in NT pro-BNP, Full analysis set

| Treatment Group | Week 12 | | | | | |
|---|---|---|---|---|---|---|
| (Subjects) | Category 1 | | Category 2 | | Missing | |
| Baseline | n (%) | | n (%) | | n (%) | |
| Macitentan 10 mg (N = 43) | | | | | | |
| ≤300 ng/L (n = 25, 58.1%) | 20 | (46.5) | 2 | (4.7) | 3 | (7.0) |
| >300 ng/L (n = 16, 37.2%) | 6 | (14.0) | 7 | (16.3) | 3 | (7.0) |
| Missing (n = 0) | 0 | | 0 | | 0 | |
| Total | 28 | (65.1) | 9 | (20.9) | 6 | (14.0) |
| Placebo (N = 42) | | | | | | |
| ≤300 ng/L (n = 29, 69.0%) | 26 | (61.9) | 3 | (7.1) | 0 | |
| >300 ng/L (n = 11, 26.2%) | 1 | (2.4) | 9 | (21.4) | 1 | (2.4) |
| Missing (n = 0) | 0 | | 0 | | 0 | |
| Total | 27 | (64.3) | 14 | (33.3) | 1 | (2.4) |

The percentages are based on N.

LT perioperative risk classification, MELD exception eligibility, and waitlist mortality analyses were performed after the database lock and SDTMs delivery for the Double-Blind period. Number (%) of patients in following categories were summarized and presented in shift tables. The estimated odds ratio for risk improvement and corresponding 95% confidence intervals were obtained from an exact logistic regression (macitentan vs placebo) with factors for treatment and risk category at baseline.

LT Perioperative Risk Classification

In approximately 88% of PoPH patients, portal hypertension is caused by liver cirrhosis. Since cirrhosis is a progressive disease, these patients will ultimately need a liver transplant, which is the only possible cure for the liver disease. Furthermore, in patients with PoPH receiving PAH-specific therapy, liver transplant has also been reported to normalize pulmonary hemodynamics and 34.8-72% of patients have been weaned off pre-transplant PAH therapy after liver transplant.

Relevance of LT Perioperative Risk Classification

The presence of moderate to severe pulmonary hypertension in patients with liver disease increases the risk of graft dysfunction and cardiopulmonary-related mortality after orthotopic liver transplant. According to the International Liver Transplant Society (ILTS) Practice Guidelines, liver transplant is conditional to appropriate hemodynamics due to the risk of cardiopulmonary-related mortality after liver transplant:
mPAP ≥45 mmHg is an absolute contraindication to liver transplant (high risk) as it is associated with 100% cardiopulmonary mortality
mPAP ≥35 mmHg and <45 mmHg, with increased PVR is associated with increased mortality (intermediate risk) of about 50%

mPAP <35 mmHg is associated with a low risk for liver transplant
LT perioperative risk classification

At baseline, most patients were in the high-risk category (25 and 17 patients in the macitentan and placebo groups, respectively) defined by a mPAP ≥45 mm Hg (Table 35). After 12 weeks of treatment, an improvement to a more favorable risk category was achieved for 18 (41.9%) and 6 (14.3%) patients in the macitentan and placebo groups, respectively. The odds to change to a more favorable risk category was 3.73 times higher in the macitentan group compared to the placebo group (95% CI 1.18, 13.40, p-value=0.0224).

TABLE 35
LT perioperative risk classification

| | Week 12 | | | | | | | |
|---|---|---|---|---|---|---|---|---|
| Treatment Group | Low | | | | High | | | |
| (Subjects) | risk | | Intermediate | | risk | | Missing | |
| Baseline | n (%) | | risk n (%) | | n (%) | | n (%) | |
| Macitentan 10 mg | | | | | | | | |
| (N = 43) | | | | | | | | |
| Low risk | 1 | (2.3) | 1 | (2.3) | 0 | | 0 | |
| (n = 2, 4.6%) | | | | | | | | |
| Intermediate risk | 5 | (11.6) | 8 | (18.6) | 0 | | 3 | (7.0) |
| (n = 1 6, 37.2%) | | | | | | | | |
| High risk | 1 | (2.3) | 12 | (27.9) | 11 | (25.6) | 1 | (2.3) |
| (n = 25, 58.1%) | | | | | | | | |
| Missing (n = 0) | 0 | | 0 | | 0 | | 0 | |
| Total | 7 | (16.3) | 21 | (48.8) | 11 | (25.6) | 4 | (9.3) |
| Placebo (N = 42) | | | | | | | | |
| Low risk | 2 | (4.8) | 2 | (4.8) | 0 | | 0 | |
| (n = 4, 9.5%) | | | | | | | | |
| Intermediate risk | 3 | (7.1) | 13 | (31.0) | 5 | (11.9) | 0 | |
| (n = 21, 50.0%) | | | | | | | | |
| High risk | 0 | | 3 | (7.1) | 13 | (31.0) | 1 | (2.4) |
| (n = 17, 40.5%) | | | | | | | | |
| Missing (n = 0) | 0 | | 0 | | 0 | | 0 | |
| Total | 5 | (11.9) | 18 | (42.9) | 18 | (42.9) | 1 | (2.4) |

| | Macitentan | | | |
|---|---|---|---|---|
| | 10 mg | | Placebo | |
| | N = 43 | | N = 42 | |
| N | 39 | | 41 | |
| Missing | 4 | (9.3) | 1 | (2.4) |
| No change | 20 | (46.5) | 28 | (66.7) |
| Improvement | 18 | (41.9) | 6 | (14.3) |
| Worsening | 1 | (2.3) | 7 | (16.7) |
| Between-treatment | | | | |
| odds ratio for | | | | |
| improvement* | | | | |

| | |
|---|---|
| OR | 3.732 |
| 95% Cl | 1.175, 13.401 |
| p-value | 0.0224 |

The percentages are based on N. Low risk if mPAP < 35 mm Hg. Intermediate risk if mPAP >= 35 and < 45 mm Hg. High risk if mPAP => 45 mm Hg.
*From exact logistic regression (macitentan vs placebo) with factors for treatment and risk category at baseline.

MELD Exception Eligibility

In general, liver grafts are attributed in priority to patients who need a liver transplant the most urgently. This assessment lies mostly on the Model for End-stage Liver Disease (MELD) score: the higher the score, the higher the patient's priority on the waiting list. In PoPH, by the time MELD score is high enough to obtain a liver graft, the hemodynamic state is generally too severely impaired for the procedure. In the US, a PoPH MELD exception rule (MELD exception) is in place to facilitate access to a liver graft to patients with PoPH who attain mPAP ≤35 mHg and PVR ≤400 dyn·sec·cm⁻⁵ with PAH treatment. Patients are priority-ranked on the waitlist with a higher MELD score than their actual score. This allows obtaining a liver graft at a time when liver impairment is moderate, before cardiopulmonary impairment becomes a contraindication to transplant.

At baseline, one patient in each treatment group met the MELD exception criteria (mPAP ≤35 mmHg and PVR ≤400 dyn·sec·cm⁻⁵; category 1 in Table 36). After 12 weeks of treatment, 8 (18.6%) macitentan patients and 2 (4.8%) placebo patients who did not meet the MELD exception criteria at baseline had hemodynamic improvements which allowed them to meet these criteria. The odds of becoming eligible for the MELD exception was 4.51 times higher for macitentan-treated than for placebo patients (95% CI 0.82, 46.65, p-value=0.0958).

TABLE 36
Shift table from baseline to Week 12 for liver transplant eligibility
based on mPAP/PVR, Full analysis set

| | Week 12 | | | | | |
|---|---|---|---|---|---|---|
| Treatment Group | Category 1 | | Category | | Missing | |
| (Subjects) Baseline | n (%) | | 2 n (%) | | n (%) | |
| Macitentan 10 mg | | | | | | |
| (N = 43) | | | | | | |
| Category 1 (n = 1, 2.3%) | 1 | (2.3) | 0 | | 0 | |
| Category 2 (n = 42, | 8 | (18.6) | 30 | (69.8) | 4 | (9.3) |
| 97.6%) | | | | | | |
| Missing (n = 0) | 0 | | 0 | | 0 | |
| Total | 9 | (20.9) | 30 | (69.8) | 4 | (9.3) |
| Placebo (N = 42) | | | | | | |
| Category 1 (n = 1, 2.4%) | 1 | (2.4) | 0 | | 0 | |
| Category 2 (n = 41, | 2 | (4.8) | 38 | (90.5) | 1 | (2.4) |
| 97.6%) | | | | | | |
| Missing (n = 0) | 0 | | 0 | | 0 | |
| Total | 3 | (7.1) | 38 | (90.5) | 1 | (2.4) |

| | Macitentan | | | |
|---|---|---|---|---|
| | 10 mg | | Placebo | |
| | N = 43 | | N = 42 | |
| N | 39 | | 41 | |
| Missing | 4 | (9.3) | 1 | (2.4) |
| No change | 31 | (72.1) | 39 | (92.9) |
| Improvement | 8 | (18.6) | 2 | (4.8) |

| | |
|---|---|
| Between-treatment odds | |
| ratio for improvement* | |
| OR | 4.512 |
| 95% Cl | 0.822, 46.462 |
| p-value | 0.0958 |

The percentages are based on N. Category 1 - mPAP ≤ 35 and PVR ≤ 400 dyn · sec/cm⁵ = YES. Category 2 - mPAP ≤ 35 and PVR ≤ 400 dyn · sec/cm⁵ = NO.
*From exact logistic regression (macitentan vs placebo) with factors for treatment and risk category at baseline.

Waitlist Mortality Risk

The outcomes of PoPH patients who were granted MELD exception in the US were analyzed. They found that removal from liver transplant waiting list due to mortality or clinical deterioration was 23.2% after a median waiting list time of 344 days. Age, initial MELD score and initial PVR were significant predictors of waitlist mortality, although their accuracy was limited. The results were illustrated by defining 4 groups depending on their PVR and MELD score: high PVR (PVR>450 dyn·sec·cm⁻⁵), low PVR (PVR ≤450 dyn·sec·cm⁻⁵), high MELD (MELD >12) or low MELD (MELD ≤12).

At baseline, 55 patients (30 macitentan and 25 placebo) had PVR >450 dyn·s·cm⁻⁵ (category 2 in Table 37), and therefore a high risk of removal from liver transplant waiting list due to mortality or clinical deterioration. After 12 weeks, 18 (41.9%) macitentan patients and 3 (7.1%) placebo patients who were initially in the waitlist high-risk group moved to the low-risk group. The odds of changing to the low waitlist mortality risk group was 10.48 times higher for the macitentan group as compared to the placebo group (95% CI 2.38, 66.81, p-value=0.0005).

TABLE 37
Shift table from baseline to Week 12 in PVR risk categories
(with odds ratio for improvement) Full analysis set

| | Week 12 | | | | | |
|---|---|---|---|---|---|---|
| Treatment Group | Category 1 | | Category 2 | | Missing | |
| (Subjects) Baseline | n (%) | | n (%) | | n (%) | |
| Macitentan 10 mg | | | | | | |
| (N = 43) | | | | | | |
| Category 1 | 11 | (25.6) | 1 | (2.3) | 1 | (2.3) |
| (n = 13, 30.2%) | | | | | | |
| Category 2 | 18 | (41.9) | 9 | (20.9) | 3 | (7.0) |
| (n = 30, 69.8%) | | | | | | |
| Missing (n = 0) | 0 | | 0 | | 0 | |
| Total | 29 | (67.4) | 10 | (23.3) | 4 | (9.3) |
| Placebo (N = 42) | | | | | | |
| Category 1 | 14 | (33.3) | 3 | (7.1) | 0 | |
| (n = 17, 40.5%) | | | | | | |
| Category 2 | 3 | (7.1) | 21 | (50.0) | 1 | (2.4) |
| (n = 25, 59.5%) | | | | | | |
| Missing (n = 0) | 0 | | 0 | | 0 | |
| Total | 17 | (40.5) | 24 | (57.1) | 1 | (2.4) |

Macitentan

| | 10 mg | | Placebo | |
|---|---|---|---|---|
| | N = 43 | | N = 42 | |
| n | 39 | | 41 | |
| Missing | 4 | (9.3) | 1 | (2.4) |
| No change | 20 | (46.5) | 35 | (83.3) |
| Improvement | 18 | (41.9) | 3 | (7.1) |
| Worsening | 1 | (2.3) | 3 | (7.1) |

| | |
|---|---|
| Between-treatment odds | |
| ratio for improvement* | |
| OR | 10.479 |
| 95% Cl | 2.381, 66.810 |
| p-value | 0.0005 |

The percentages are based on N. Category 1 - PVR <= 450 dyn · sec/cm⁵. Category 2 - PVR > 450 dyn · sec/cm⁵.
*From exact logistic regression (macitentan vs placebo) with factors for treatment and risk category at baseline.

Other Variables

At Week 12, patients receiving macitentan had a mean PAWP/LVEDP (SD) of 11.4 (4.2) mmHg with a mean (SD) change from baseline of 2.1 (3.7) mmHg. Mean PAWP/LVEDP (SD) was 9.4 (3.1) mmHg at Week 12 and mean (SD) change from baseline was −0.3 (3.4) mmHg in the placebo group. The treatment difference based on the change from baseline to Week 12 was 2.4 (95% CI: 0.8, 4.0) mmHg.

D. Conclusions

During the DB phase, macitentan increased the likelihood to observe a relevant decrease in NT-proBNP. Based on hemodynamics, macitentan increased the likelihood to obtain a liver graft and to be in a low risk category for liver transplant perioperative mortality. It also decreased the risk for the patients to be in a high waiting list mortality risk category. These results were obtained despite a high proportion of patients receiving a PAH-specific therapy at baseline. These analyses suggest that macitentan treatment may be of beneficial value for PoPH patients who may need a liver transplant.

### Example 3—Post-Hoc Analyses

This example provides post hoc analyses of the study performed in Example 1. These analyses include:
To assess the effect of macitentan on mean pulmonary arterial pressure (mPAP) related to pre-treatment mPAP.
To assess the effect of macitentan on pulmonary vascular resistance (PVR) related to pre-treatment PVR.

The following exploratory endpoints were defined for the post hoc analyses:
mPAP change related to pre-treatment mPAP.
PVR change related to pre-treatment PVR

A. Statistical Methods

All analyses were performed on the full analysis set (FAS, as per randomization, N=85) and no data imputation was performed. The FAS included all randomized patients who received at least one dose of study treatment in the double-blind treatment period and had a baseline value for the primary endpoint of PVR.

Risk categories are noted in Table 38. To analyze change in mPAP relative to baseline mPAP, the same mPAP categories were used as for transplant perioperative risk classification (Table 38). For the analysis of change in PVR relative to baseline PVR, waitlist mortality risk PVR categories were used (Table 38).

TABLE 38
Risk Categories Used for Analyses

| Analysis | Categories |
|---|---|
| Transplant | Low risk: mPAP < 35 mmHg |
| perioperative risk | Intermediate risk: mPAP ≥ 35 and < 45 mmHg |
| classification | High risk: mPAP ≥ 45 mmHg |
| | Missing |
| MELD exception | Category 1: mPAP ≤ 35 and PVR ≤ 400 |
| eligibility | dyn · s/cm⁵ = YES (eligible) |
| | Category 2: mPAP ≤ 35 and PVR ≤ 400 |
| | dyn · s/cm⁵ = NO (not eligible) |
| | Missing |
| Waitlist mortality | Category 1: PVR ≤ 450 dyn · s/cm⁵ (low risk) |
| risk | Category 2: PVR > 450 dyn · s/cm⁵ (high risk) |
| | Missing |

B. Results

(i) Patient Disposition

There were 85 patients (43 macitentan, 42 placebo) in the FAS and all were included in the post hoc analyses. All patients in the study had a hepatic cause of portal hypertension, although this was not an entry criterion.

(ii) Exposure to Study Treatment

The median duration of double-blind treatment exposure was approximately 12 weeks and was similar for macitentan and placebo groups. 90.7% macitentan-treated patients and 95.2% placebo-treated patients received treatment until the start of the Week 12 visit window.

(iii) Rationale and Results of Post Hoc Efficacy Analyses

Extent of Change in mPAP

The extent of the change in mPAP was investigated to determine whether the magnitude of the change is related to the pre-treatment value.

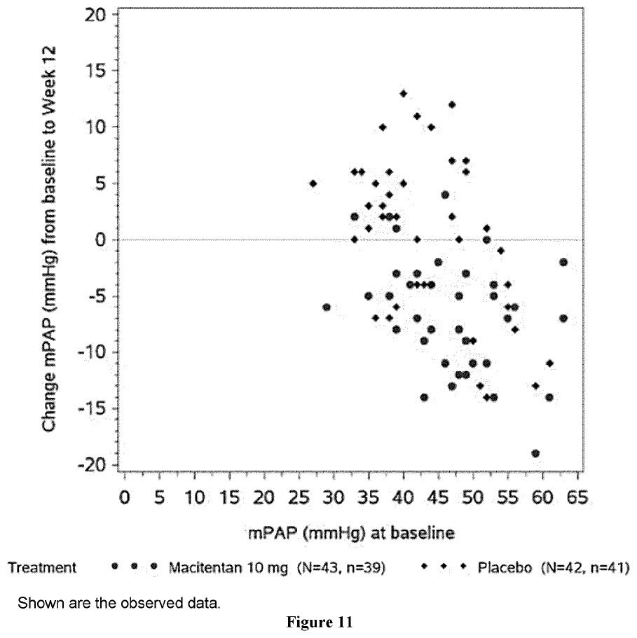

Thirty-eight (88.3%) macitentan-treated patients had a decrease in mPAP at Week 12 compared to baseline, with a trend toward a greater decrease in patients with higher baseline mPAP (FIG. 11). In the macitentan group, the mean (standard deviation [SD]) decrease in mPAP at Week 12 was 7.8 (5.27) mmHg in patients with baseline mPAP ≥45 mmHg, 5.2 (4.22) mmHg in patients with baseline mPAP 235 mmHg and <45 mmHg, and 2.0 (5.66) mmHg in patients with baseline mPAP <35 mmHg (Table 39).

TABLE 39
Change in mPAP by Baseline mPAP Category, Full Analysis Set

| | Macitentan | |
|---|---|---|
| | 10 mg | Placebo |
| mPAP at baseline < 35 mmHg | | |
| mPAP (mmHg) Baseline | | |
| n | 2 | 4 |
| Mean | 31.0 | 31.8 |
| SD | 2.83 | 3.20 |
| Median | 31.0 | 33.0 |
| Q1, Q3 | 29.0, 33.0 | 30.0, 33.5 |
| Min, Max | 29, 33 | 27, 34 |
| Change from baseline to Week 12 | | |
| n | 2 | 4 |
| Mean | −2.0 | 4.3 |
| SD | 5.66 | 2.87 |
| Median | −2.0 | 5.5 |
| Q1, Q3 | −6.0, 2.0 | 2.5, 6.0 |
| Min, Max | −6, 2 | 0, 6 |
| mPAP at baseline ≥ 35 to < 45 mmHg | | |
| mPAP 9 mmHg Baseline | | |
| n | 13 | 21 |
| Mean | 40.5 | 38.9 |
| SD | 2.76 | 2.96 |
| Median | 41.0 | 38.0 |
| Q1, Q3 | 39.0, 43.0 | 37.0, 42.0 |
| Min, Max | 35, 44 | 35, 44 |
| Change from baseline to Week 12 | | |
| n | 13 | 21 |
| Mean | −5.2 | 2.1 |
| SD | 4.22 | 5.96 |
| Median | −5.0 | 2.0 |
| Q1, Q3 | −8.0, −3.0 | −4.0, 5.0 |
| Min, Max | −14, 2 | −7, 13 |
| mPAP at baseline ≥ 45 mmHg | | |
| mPAP (mmHg) Baseline | | |
| n | 24 | 16 |
| Mean | 51.8 | 52.0 |
| SD | 5.36 | 4.34 |
| Median | 51.0 | 51.5 |
| Q1, Q3 | 48.0, 54.0 | 48.5, 55.0 |
| Min, Max | 45, 63 | 47, 61 |
| Change from baseline to Week 12 | | |
| n | 24 | 16 |
| Mean | −7.8 | −2.8 |
| SD | 5.27 | 8.23 |
| Median | −7.5 | −2.5 |
| Q1, Q3 | −11.5, −4.5 | −10.0, 4.0 |
| Min, Max | −19, 4 | −14, 12 |

Modified from Table 39A

TABLE 39A
mPAP Change From Baseline at Week 12 by Baseline mPAP
Categories, Full Analysis Set

| | Macitentan | Placebo |
|---|---|---|
| | 10 mg | |
| | N = 2 | N = 4 |
| mPAP at baseline < 35 mmHg | | |
| mPAP (mmHg) Baseline | | |
| n | 2 | 4 |
| Mean | 31.0 | 31.8 |
| SD | 2.83 | 3.20 |
| Median | 31.0 | 33.0 |
| Q1, Q3 | 29.0, 33.0 | 30.0, 33.5 |
| Min, Max | 29, 33 | 27, 34 |
| Week 12 | | |
| n | 2 | 4 |
| Mean | 29.0 | 36.0 |
| SD | 8.49 | 4.08 |
| Median | 29.0 | 36.0 |
| Q1, Q3 | 23.0, 35.0 | 32.5, 39.5 |
| Min, Max | 23, 35 | 32, 40 |
| Change from baseline to Week 12 | | |
| n | 2 | 4 |
| Mean | −2.0 | 4.3 |
| SD | 5.66 | 2.87 |
| Median | −2.0 | 5.5 |
| Q1, Q3 | −6.0, 2.0 | 2.5, 6.0 |
| Min, Max | −6, 2 | 0, 6 |
| mPAP at baseline 35 to < 45 mmHg | | |
| mPAP (mmHg) Baseline | | |
| n | 13 | 21 |
| Mean | 40.5 | 38.9 |
| SD | 2.76 | 2.96 |
| Median | 41.0 | 38.0 |
| Q1, Q3 | 39.0, 43.0 | 37.0, 42.0 |
| Min, Max | 35, 44 | 35, 44 |
| Week 12 | | |
| n | 13 | 21 |
| Mean | 35.4 | 41.0 |
| SD | 3.84 | 6.73 |
| Median | 36.0 | 40.0 |
| Q1, Q3 | 33.0, 39.0 | 38.0, 44.0 |
| Min, Max | 29, 40 | 29, 54 |
| Change from baseline to Week 12 | | |
| n | 13 | 21 |
| Mean | −5.2 | 2.1 |
| SD | 4.22 | 5.96 |
| Median | −5.0 | 2.0 |
| Q1, Q3 | −8.0, −3.0 | −4.0, 5.0 |
| Min, Max | −14, 2 | −7, 13 |
| mPAP at baseline >= 45 mmHg | | |
| mPAP (mmHg) Baseline | | |
| n | 24 | 16 |
| Mean | 51.8 | 52.0 |
| SD | 5.36 | 4.34 |
| Median | 51.0 | 51.5 |
| Q1, Q3 | 48.0, 54.0 | 48.5, 55.0 |
| Min, Max | 45, 63 | 47, 61 |
| Week 12 | | |
| n | 24 | 16 |
| Mean | 44.0 | 49.3 |
| SD | 7.04 | 6.13 |
| Median | 43.0 | 49.5 |
| Q1, Q3 | 39.0, 48.5 | 47.0, 53.5 |
| Min, Max | 34, 61 | 38, 59 |
| Change from baseline to Week 12 | | |
| n | 24 | 16 |
| Mean | −7.8 | −2.8 |
| SD | 5.27 | 8.23 |
| Median | −7.5 | −2.5 |
| Q1, Q3 | −11.5, −4.5 | −10.0, 4.0 |
| Min, Max | −19, 4 | −14, 12 |

Extent of Change in PVR

The extent of the change in PVR was investigated to determine whether the magnitude of the change is related to the pre-treatment value.

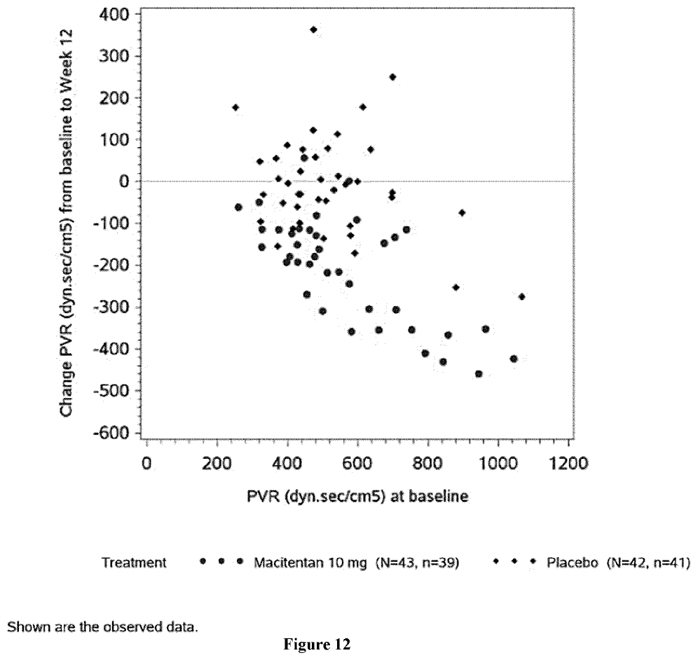

As shown in FIG. 12, 41 (95.3%) macitentan-treated patients had a decrease in PVR at Week 12 compared to baseline, with a trend toward a greater decrease in patients with higher baseline PVR. In the macitentan group, the mean (SD) decrease in PVR was 249.4 (125.48) dyn·s/cm⁵ and 116.4 (71.23) dyn·s/cm⁵ in patients with baseline PVR >450 dyn·s/cm⁵ and PVR <450 dyn·s/cm⁵, respectively, at baseline (Table 40).

TABLE 40
Change in PVR by Baseline PVR Category, Full Analysis Set

| | Macitentan | |
|---|---|---|
| | 10 mg | Placebo |
| PVR at baseline <= 450 dyn · sec/cm⁵ | | |
| PVR (dyn · sec/cm⁵) Baseline | | |
| n | 12 | 17 |
| Mean | 381.0 | 386.3 |
| SD | 58.56 | 53.44 |
| Median | 402.8 | 400.0 |
| Q1, Q3 | 328.1, 429.1 | 368.0, 432.4 |
| Min, Max | 261, 448 | 253, 444 |
| Change from baseline to Week 12 | | |
| n | 12 | 17 |
| Mean | −116.4 | −12.1 |
| SD | 71.23 | 84.20 |
| Median | −120.0 | −31.0 |
| Q1, Q3 | −168.4, −87.2 | −61.4, 47.3 |
| Min, Max | −193, 56 | −155, 177 |
| PVR at baseline > 450 dyn · sec/cm⁵ | | |
| PVR (dyn · sec/cm⁵) Baseline | | |
| n | 27 | 24 |
| Mean | 650.1 | 611.6 |
| SD | 170.76 | 150.54 |
| Median | 598.1 | 572.6 |
| Q1, Q3 | 490.5, 754.7 | 506.7, 667.8 |
| Min, Max | 456, 1046 | 474, 1069 |
| Change from baseline to Week 12 | | |
| n | 27 | 24 |
| Mean | −249.4 | −3.1 |
| SD | 125.48 | 147.11 |
| Median | −244.9 | −14.1 |
| Q1, Q3 | −355.0, −133.4 | −90.4, 77.9 |
| Min, Max | −459, 1 | −276, 364 |

Modified from Table 40A.

TABLE 40A
PVR Change From Baseline at Week 12 by Baseline PVR
Categories, Full Analysis Set

| | Macitentan | |
|---|---|---|
| | 10 mg | Placebo |
| | N =12 | N =17 |
| PVR at baseline <= 450 dyn · sec/cm⁵ | | |
| PVR (dyn · sec/cm⁵) Baseline | | |
| n | 12 | 17 |
| Mean | 381.0 | 386.3 |
| SD | 58.56 | 53.44 |
| Median | 402.8 | 400.0 |
| Q1, Q3 | 328.1, 429.1 | 368.0, 432.4 |
| Min, Max | 261, 448 | 253, 444 |
| Week 12 | | |
| n | 12 | 17 |
| Mean | 264.6 | 374.2 |
| SD | 86.64 | 82.49 |
| Median | 248.7 | 380.3 |
| Q1, Q3 | 209.7, 282.7 | 335.3, 422.5 |
| Min, Max | 171, 504 | 218, 521 |
| Change from baseline to Week 12 | | |
| n | 12 | 17 |
| Mean | −116.4 | −12.1 |
| SD | 71.23 | 84.20 |
| Median | −120.0 | −31.0 |
| Q1, Q3 | −168.4, −87.2 | −61.4, 47.3 |
| Min, Max | −193, 56 | −155, 177 |
| PVR at baseline > 450 dyn · sec/cm⁵ | | |
| PVR (dyn · sec/cm⁵) Baseline | | |
| n | 27 | 24 |
| Mean | 650.1 | 611.6 |
| SD | 170.76 | 150.54 |
| Median | 598.1 | 572.6 |
| Q1, Q3 | 490.5, 754.7 | 506.7, 667.8 |
| Min, Max | 456, 1046 | 474, 1069 |
| Week 12 | | |
| n | 27 | 24 |
| Mean | 400.7 | 608.6 |
| SD | 130.62 | 150.25 |
| Median | 382.5 | 595.7 |
| Q1, Q3 | 305.9, 506.3 | 486.7, 693.0 |
| Min, Max | 186, 625 | 367, 950 |
| Change from baseline to Week 12 | | |
| n | 27 | 24 |
| Mean | −249.4 | −3.1 |
| SD | 125.48 | 147.11 |
| Median | −244.9 | −14.1 |
| Q1, Q3 | −355.0, −133.4 | −90.4, 77.9 |
| Min, Max | −459, 1 | −276, 364 |

1. A method for treating portopulmonary hypertension, comprising administering to a patient in need thereof, a therapeutically effective amount of macitentan.

2. The method of claim 1, wherein, before the administration of macitentan, the patient has a mean pulmonary arterial hypertension (mPAP) of >25 mmHg at rest, a mean pulmonary arterial wedge pressure 15 mmHg, and a pulmonary vascular resistance (PVR) >3 WU (Wood Units).

3. The method of claim 1, wherein, before the administration of macitentan, the patient has a pulmonary vascular resistance (PVR) of ≥4 Wood units (≥320 dyn·s·cm⁻⁵) and is capable of performing a 6-minute walk test with a distance ≥50 m.

4. The method of claim 1, wherein, before the administration of macitentan, the patient does not have Child-Pugh class C liver disease or a Model for End-Stage Liver Disease (MELD) score of ≥19.

5. The method of claim 1, wherein the patient receives background therapy comprising phosphodiesterase type 5 inhibitors, soluble guanylate cyclase stimulators, or inhaled prostanoids, wherein such therapy is present for at least three months at a stable dose prior to administration of macitentan.

6. The method of claim 1, wherein the method reduces PVR.

7. The method of claim 6, wherein the PVR is reduced by at least about 30% relative to a patient at the same level of disease diagnosis that is not receiving treatment with macitentan.

8. The method of claim 1, wherein the administration of macitentan does not substantially affect hepatic venous pressure gradient or systolic blood pressure of the patient.

9. The method of claim 1, wherein mean pulmonary arterial pressure and total pulmonary resistance are reduced following the administration of macitentan.

10. The method of claim 1, wherein cardiac index is increased following administration of macitentan.

11. The method of claim 1, wherein the portopulmonary hypertension is caused by liver disease.

12. The method of claim 1, wherein the therapeutically effective amount of macitentan is about 5 to about 15 mg.

13. The method of claim 12, wherein the therapeutically effective amount of macitentan is about 10 mg.

14. The method of claim 1, wherein the macitentan is administered orally in the form of a tablet once daily.

15. (canceled)

16. A method of improving liver transplant perioperative mortality risk category in a patient with portopulmonary hypertension and liver disease, comprising administering to a patient in need thereof, a therapeutically effective amount of macitentan.

17. The method of claim 16, wherein, before the administration of macitentan, the patient has a mPAP ≥45 mmHg and a liver transplant is contraindicated.

18. The method of claim 16, wherein, before the administration of macitentan, the patient has a mPAP ≥35 mmHg and <45 mmHg.

19. The method of claim 16, wherein, before the administration of macitentan, the patient has a mPAP <35 mmHg.

20. The method of claim 17, wherein the method results in a mPAP ≥35 mmHg and <45 mmHg.

21. The method of claim 17, wherein the method results in a mPAP <35 mmHg.

22. The method of claim 18, wherein the method results in a mPAP <35 mmHg.

23. A method of improving MELD exception eligibility in a patient with portopulmonary hypertension and liver disease, comprising administering to a patient in need thereof, a therapeutically effective amount of macitentan.

24. The method of claim 23, wherein the MELD exception eligibility criteria comprises a mPAP ≤35 mmHg and a PVR ≤400 dyn·sec·cm⁻⁵.

25. The method of claim 24, wherein the method results in a patient outside the MELD exception eligibility criteria meeting the MELD exception eligibility criteria.

26. The method of claim 25, wherein the method allows the patient to obtain a liver graft.

27. A method of reducing the risk of removal from a liver transplant waitlist due to mortality or clinical deterioration in a patient with portopulmonary hypertension and liver disease, comprising administering to a patient in need thereof, a therapeutically effective amount of macitentan.

28. The method of claim 27, wherein, before the administration of macitentan, the patient has a PVR >450 dyn·s·cm⁻⁵ and a high risk of removal from the liver transplant waitlist.

29. The method of claim 28, wherein the method results in the patient reducing the risk of removal from the liver transplant waitlist by lowering the PVR to 400 dyn·sec·cm⁻⁵.

30. The method of claim 16, wherein the method comprises about twelve weeks of treatment with the macitentan.

31. The method of claim 16, wherein the patient is receiving background pulmonary arterial hypertension (PAH) specific therapy.

32. The method of claim 31, wherein the background PAH specific therapy comprises a phosphodiesterase type 5 inhibitor, a soluble guanylate cyclase stimulator, or an inhaled prostanoid.

33. The method of claim 31, wherein the background PAH specific therapy is present for at least three months at a stable dose prior to administration of macitentan.

34. The method of claim 16, wherein the administration of macitentan does not substantially affect hepatic venous pressure gradient.

35. The method of claim 16, wherein the portopulmonary hypertension is caused by cirrhosis or Hepatitis C.

36. The method of claim 16, wherein the therapeutically effective amount of macitentan is about 5 to about 15 mg.

37. The method of claim 36, wherein the therapeutically effective amount of macitentan is about 10 mg.

38. The method of claim 14, wherein the macitentan is administered orally in the form of a tablet once daily.

39-48. (canceled)

49. A pharmaceutical drug product comprising a therapeutically effective amount of macitentan, wherein the pharmaceutical product is packaged and wherein the package includes a label that identifies macitentan as a regulatory approved chemical entity, and instructs use of macitentan for the treatment of portopulmonary hypertension.

50. The pharmaceutical product of claim 49, wherein the label includes data comprises data for reducing PVR relative to a placebo.

51. The pharmaceutical drug product of claim 49, wherein the drug product label comprises data or instructions for improving liver transplant perioperative mortality risk category, improving MELD exception eligibility, or reducing the risk of removal from a liver transplant waitlist.

52-90. (canceled)
